SEQUENCE LISTING (7 sequences)

SEQ ID NO: 1 - protein, 543 aa, Homo sapiens
MAKGEGAESGSAAGLLPTSILQSTERPAQVKKEPKKKKQQLSVCNKLCYALGGAPYQVTGCALGFFLQIYLLDVAQKDEEVVFCFSSFQVGPFSASIILFVGRAWDAITDPLVGLCISKSPWTCLGRLMPWIIFSTPLAVIAYFLIWFVPDFPHGQTYWYLLFYCLFETMVTCFHVPYSALTMFISTEQTERDSATAYRMTVEVLGTVLGTAIQGQIVGQADTPCFQDLNSSTVASQSANHTHGTTSHRETQKAYLLAAGVIVCIYIICAVILILGVREQREPYEAQQSEPIAYFRGLRLVMSHGPYIKLITGFLFTSLAFMLVEGNFVLFCTYTLGFRNEFQNLLLAIMLSATLTIPIWQWFLTRFGKKTAVYVGISSAVPFLILVALMESNLIITYAVAVAAGISVAAAFLLPWSMLPDVIDDFHLKQPHFHGTEPIFFSFYVFFTKFASGVSLGISTLSLDFAGYQTRGCSQPERVKFTLNMLVTMAPIVLILLGLLLFKMYPIDEERRRQNKKALQALRDEASSSGCSETDSTELASIL

SEQ ID NO: 2 - DNA, 2236 bp, Homo sapiens
agaactataagaggcgcggagggggcgtgcagcagagtgcgttcctcgtctgccagccggcttggctagcgcgcggcggccgtggctaaggctgctacgaagcgagcttgggaggagcagcggcctgcggggcagaggagcatcccgtctaccaggtcccaagcggcgtggcccgcgggtcatggccaaaggagaaggcgccgagagcggctccgcggcggggctgctacccaccagcatcctccaaagcactgaacgcccggcccaggtgaagaaagaaccgaaaaagaagaaacaacagttgtctgtttgcaacaagctttgctatgcacttgggggagccccctaccaggtgacgggctgtgccctgggtttcttccttcagatctacctattggatgtggctcagaaggatgaggaagttgtcttttgcttctcctcattccaggtgggccctttctctgcctccatcatcctgtttgtgggccgagcctgggatgccatcacagaccccctggtgggcctctgcatcagcaaatccccctggacctgcctgggtcgccttatgccctggatcatcttctccacgcccctggccgtcattgcctacttcctcatctggttcgtgcccgacttcccacacggccagacctattggtacctgcttttctattgcctctttgaaacaatggtcacgtgtttccatgttccctactcggctctcaccatgttcatcagcaccgagcagactgagcgggattctgccaccgcctatcggatgactgtggaagtgctgggcacagtgctgggcacggcgatccagggacaaatcgtgggccaagcagacacgccttgtttccaggacctcaatagctctacagtagcttcacaaagtgccaaccatacacatggcaccacctcacacagggaaacgcaaaaggcatacctgctggcagcgggggtcattgtctgtatctatataatctgtgctgtcatcctgatcctgggcgtgcgggagcagagagaaccctatgaagcccagcagtctgagccaatcgcctacttccggggcctacggctggtcatgagccacggcccatacatcaaacttattactggcttcctcttcacctccttggctttcatgctggtggaggggaactttgtcttgttttgcacctacaccttgggcttccgcaatgaattccagaatctactcctggccatcatgctctcggccactttaaccattcccatctggcagtggttcttgacccggtttggcaagaagacagctgtatatgttgggatctcatcagcagtgccatttctcatcttggtggccctcatggagagtaacctcatcattacatatgcggtagctgtggcagctggcatcagtgtggcagctgccttcttactaccctggtccatgctgcctgatgtcattgacgacttccatctgaagcagccccacttccatggaaccgagcccatcttcttctccttctatgtcttcttcaccaagtttgcctctggagtgtcactgggcatttctaccctcagtctggactttgcagggtaccagacccgtggctgctcgcagccggaacgtgtcaagtttacactgaacatgctcgtgaccatggctcccatagttctcatcctgctgggcctgctgctcttcaaaatgtaccccattgatgaggagaggcggcggcagaataagaaggccctgcaggcactgagggacgaggccagcagctctggctgctcagaaacagactccacagagctggctagcatcctctagggcccgccacgttgcccgaagccaccatgcagaaggccacagaagggatcaggacctgtctgccggcttgctgagcagctggactgcaggtgctaggaagggaactgaagactcaaggaggtggcccaggacacttgctgtgctcactgtggggccggctgctctgtggcctcctgcctcccctctgcctgcctgtggggccaagccctggggctgccactgtgaatatgccaaggactgatcgggcctagcccggaacactaatgtagaaacctttttttttacagagcctaattaataacttaatgactgtgtacatagcaatgtgtgtgtatgtatatgtctgtgagctattaatgttattaattttcataaaagctggaaagcaaaaaaaaaaaaaaaaaaaaaaaaaaaaaaaaaaaa

SEQ ID NO: 3 - protein, 533 aa, Mus sp.
MAKGEGAESGSAAGLLPTSILQASERPVQVKKEPKKKQQLSICNKLCYAVGGAPYQLTGCALGFFLQIYLLDVAKVEPLPASIILFVGRAWDAFTDPLVGFCISKSSWTRLGRLMPWIIFSTPLAIIAYFLIWFVPDFPSGTESSHGFLWYLLFYCLFETLVTCFHVPYSALTMFISTEQSERDSATAYRMTVEVLGTVIGTAIQGQIVGQAKAPCLQDQNGSVVVSEVANRTQSTASLKDTQNAYLLAAGIIASIYVLCAFILILGVREQRELYESQQAESMPFFQGLRLVMGHGPYVKLIAGFLFTSAFMLVEGNFALFCTYTLDFRNEFQNLLLAIMLSATFTIPIWQWFLTRFGKKTAVYIGISSAVPFLILVALMERNLIVTYVVAVAAGVSVAAAFLLPWSMLPDVIDDFHLKHPHSPGTEPIFFSFYVFFTKFASGVSLGVSTLSLDFANYQRQGCSQPEQVKFTLKMLVTMAPIILILLGLLLFKLYPIDEEKRRQNKKALQALREEASSSGCSDTDSTELASIL

SEQ ID NO: 4 - DNA, 21 bp, Artificial Sequence
  Description of Artificial Sequence Synthetic primer
cctggtttgctaagtgctagc

SEQ ID NO: 5 - DNA, 21 bp, Artificial Sequence
  Description of Artificial Sequence Synthetic primer
gttcactggcttggaggatgc

SEQ ID NO: 6 - DNA, 21 bp, Artificial Sequence
  Description of Artificial Sequence Synthetic primer
cacttcctaaagccttacttc

SEQ ID NO: 7 - DNA, 20 bp, Artificial Sequence
  Description of Artificial Sequence Synthetic primer
gcagcgcatcgccttctatc

(19) United States
(12) Patent Application Publication
(10) Pub. No.: US 2016/0120893 A1
(43) Pub. Date: May 5, 2016

(54) METHODS AND COMPOSITIONS RELATING TO MODULATION OF THE PERMEABILITY OF THE BLOOD BRAIN BARRIER

(71) Applicant: PRESIDENT AND FELLOWS OF HARVARD COLLEGE, Cambridge, MA (US)

(72) Inventors: Chenghua GU, Newton, MA (US); Ayal BEN-ZVI, Brookline, MA (US)

(73) Assignee: PRESIDENT AND FELLOWS OF HARVARD COLLEGE, Cambridge, MA (US)

(21) Appl. No.: 14/897,264

(22) PCT Filed: Jun. 20, 2014
(86) PCT No.: PCT/US14/43395

§ 371 Date: Dec. 10, 2015

Related U.S. Application Data
(60) Provisional application No. 61/837,782, filed on Jun. 21, 2013.
(60) Provisional application No. 61/839,059, filed on Jun. 25, 2013.
(60) Provisional application No. 61/876,406, filed on Sep. 11, 2013.
(60) Provisional application No. 61/912,637, filed on Dec. 6, 2013.

(51) Int. Cl. A61K 31/7072; A61K 45/06

(52) U.S. Cl. CPC A61K 31/7072; A61K 45/06

(57) ABSTRACT

Described herein are methods and compositions relating to modulating the permeability of the blood-brain barrier, e.g. increasing or decreasing the permeability of the blood-brain barrier for therapeutic purposes.

### CROSS-REFERENCE TO RELATED APPLICATIONS

This application claims benefit under 35 U.S.C. §119(e) of U.S. Provisional Application Nos. 61/837,782 filed Jun. 21, 2013, 61/839,059 filed Jun. 25, 2013, 61/876,406 filed Sep. 11, 2013, and 61/912,637 filed Dec. 6, 2013, the contents of which are incorporated herein by reference in their entirety.

### SEQUENCE LISTING

The instant application contains a sequence listing which has been submitted electronically in ascii format and is hereby incorporated by reference in its entirety. Said ascii copy, created on Jun. 20, 2014, is named 002806-075954-PCT_SL.txt and is 13,651 bytes in size

### TECHNICAL FIELD

The technology described herein relates to methods of modulation of the blood brain barrier, e.g. loosening or strengthening the blood brain barrier.

### BACKGROUND

The central nervous system (CNS) functions in a tightly controlled and stable environment. This is maintained by highly specialized blood vessels that physically seal the CNS and control substance influx/efflux, known as the ‘blood brain barrier’ (BBB). Specialized tight junctions between endothelial cells comprising a single layer that lines the CNS capillaries are the physical seal between blood and brain (Daneman et al. Nature. 2010 468:562-566; Armulik et al. Nature 2010 468:557-561). BBB selectivity is facilitated by an array of endothelial transporters responsible for the supply of nutrients and for the clearance of waste or toxins (Bell et al. Neuron 2010 68:409427). In concert with pericytes and astrocytes, the BBB protects the brain from various toxins and pathogens and provides the proper chemical composition for synaptic transmissions. Therefore, proper function of the CNS critically depends on BBB integrity.

Emerging evidence shows that BBB breakdown occurs in many neurodegenerative diseases prior to noticeable neuronal abnormalities. On the other hand, the BBB is also a major obstacle for drug delivery to the CNS, approximately 98% of small molecules and most large molecules/biologics can not freely pass through the BBB. Therefore, largely unsuccessful attempts have been made, both to “loosen” the BBB for drugs to pass through and to “re-seal” the BBB to treat various CNS disorders.

### SUMMARY

The inventors, through use of a new assay for examining the development of the blood brain barrier (BBB), have discovered that certain genes (e.g. Mfsd2A) are key regulators of the development of the interactions that are critical to the integrity of the BBB. Accordingly, described herein are methods of modulating the BBB by inhibiting or increasing the activity of these genes. Inhibiting, e.g., Mfsd2A, and thereby loosening the BBB, can allow drugs to be more readily delivered to the central nervous system. Increasing, e.g., Mfsd2A activity, and thereby strengthening the BBB, can permit the treatment of a number of neurodegenerative diseases.

In one aspect, described herein is a method of modulating the permeability of the blood-brain barrier in a subject, the method comprising administering an inhibitor (e.g. antagonist or binder) of a gene selected from the group consisting of Mfsd2A; Slco1C1; Slc38A5; LRP8; Slc3A2; Slc7A5; Slc7A1; Slc6A6; IGFBP7; Glut1; Slc40A1; and Slc30A1 to the subject, whereby the permeability of the blood-brain barrier is increased or administering an agonist of a gene selected from the group consisting of Mfsd2A; Slco1C1; Slc38A5; LRP8; Slc3A2; Slc7A5; Slc7A1; Slc6A6; IGFBP7; Glut1; Slc40A1; and Slc30A1 to the subject, whereby the permeability of the blood-brain barrier is decreased. In one aspect, described herein is a method of treatment, the method comprising administering an inhibitor of a gene selected from the group consisting of Mfsd2A; Slco1C1; Slc38A5; LRP8; Slc3A2; Slc7A5; Slc7A1; Slc6A6; IGFBP7; Glut1; Slc40A1; and Slc30A1 to a subject in need of increased permeability of the blood-brain barrier or administering an agonist of a gene selected from the group consisting of Mfsd2A; Slco1C1; Slc38A5; LRP8; Slc3A2; Slc7A5; Slc7A1; Slc6A6; IGFBP7; Glut1; Slc40A1; and Slc30A1 to the subject in need of decreased permeability of the blood-brain barrier. In one aspect, described herein is a method of modulating the permeability of the blood-brain barrier in a subject, the method comprising administering an inhibitor of Mfsd2A to the subject, whereby the permeability of the blood-brain barrier is increased or administering an agonist of Mfsd2A to the subject, whereby the permeability of the blood-brain barrier is decreased. In one aspect, described herein is a method of treatment, the method comprising administering an inhibitor of Mfsd2A to a subject in need of increased permeability of the blood-brain barrier or administering an agonist of Mfsd2A to the subject in need of decreased permeability of the blood-brain barrier.

In some embodiments, the inhibitor is selected from the group consisting of inhibitory antibodies and inhibitory nucleic acids. In some embodiments, the subject administered an inhibitor is in need of delivery of a central nervous system therapeutic agent to the central nervous system. In some embodiments, the inhibitor of Mfsd2A is selected from the group consisting of tunicamycin; tunicamycin analogs; inhibitory anti-Mfsd2A antibodies; and inhibitory nucleic acids. In some embodiments, the subject administered an inhibitor of Mfsd2A is in need of delivery of a central nervous system therapeutic agent to the central nervous system. In some embodiments, the method further comprises administering a central nervous system therapeutic agent to the subject. In some embodiments, the subject in need of increased permeability of the blood-brain barrier is in need of treatment for a condition selected from the group consisting of brain cancer; encephalitis; hydrocephalus; Parksinson's disease; neuropathic pain; and a condition treated by the administration of psychiatric drugs.

In some embodiments, the agonist is selected from the group consisting of a polypeptide and a nucleic acid encoding a polypeptide selected from the group consisting of Mfsd2A; Slco1C1; Slc38A5; LRP8; Slc3A2; Slc7A5; Slc7A1; Slc6A6; IGFBP7; Glut1; Slc40A1; and Slc30A1. In some embodiments, the subject administered an agonist is in need of improved quality of tight junctions of the blood-brain barrier. In some embodiments, the agonist of Mfsd2A is selected from the group consisting of a Mfsd2A polypeptide; and a nucleic acid encoding a Mfsd2A polypeptide. In some embodiments, the subject administered an agonist of Mfsd2A is in need of improved quality of tight junctions of the blood-brain barrier. In some embodiments, the subject in need of decreased permeability of the blood-brain barrier is in need of treatment for a condition selected from the group consisting of a neurodegenerative disease; multiple sclerosis; Parkinson's disease; Huntington's disease; Pick's disease; ALS; dementia; stroke; and Alzheimer's disease.

In one aspect, described herein is a pharmaceutical composition comprising an inhibitor of a gene selected from the group consisting of Mfsd2A; Slco1C1; Slc38A5; LRP8; Slc3A2; Slc7A5; Slc7A1; Slc6A6; IGFBP7; Glut1; Slc40A1; and Slc30A1 and a pharmaceutically-acceptable carrier. In some embodiments, the inhibitor is selected from the group consisting of inhibitory antibodies and inhibitory nucleic acids. In one aspect, described herein is a pharmaceutical composition comprising an inhibitor of Mfsd2A and a pharmaceutically-acceptable carrier. In some embodiments, the inhibitor of Mfsd2A is selected from the group consisting of tunicamycin; tunicamycin analogs; inhibitory anti-Mfsd2A antibodies; inhibitory and nucleic acids. In some embodiments, the composition can further comprise a central nervous system therapeutic agent.

In one aspect, described herein is a pharmaceutical composition comprising an agonist of a gene selected from the group consisting of Mfsd2A; Slco1C1; Slc38A5; LRP8; Slc3A2; Slc7A5; Slc7A1; Slc6A6; IGFBP7; Glut1; Slc40A1; and Slc30A1 and a pharmaceutically-acceptable carrier. In some embodiments, the agonist is selected from the group consisting of a polypeptide and a nucleic acid encoding a polypeptide selected from the group consisting of Mfsd2A; Slco1C1; Slc38A5; LRP8; Slc3A2; Slc7A5; Slc7A1; Slc6A6; IGFBP7; Glut1; Slc40A1; and Slc30A1. In one aspect, described herein is a pharmaceutical composition comprising an agonist of Mfsd2A and a pharmaceutically-acceptable carrier. In some embodiments, the agonist of Mfsd2A is selected from the group consisting of a Mfsd2A polypeptide; and a nucleic acid encoding a Mfsd2A polypeptide.

In one aspect, described herein is a method for determining the permeability of the blood-brain barrier during development, the method comprising injecting the liver of an embryo with a detectable agent while the embryo is connected to the maternal circulation via the umbilical cord, allowing the dye to circulate in the bloodstream, and detecting a signal from the detectable agent in blood vessels within the brain and within brain tissue separated from the bloodstream by the blood-brain barrier. In some embodiments, the agent is a fixable dye. In some embodiments, the total volume of the injection is less than or equal to 1 uL for a murine embryo of about 13.5 days age, less than or equal to 2 uL for a murine embryo of about 14.5 days of age, and less than or equal to 5 uL for a murine embryo of about 15 days of age or older. In some embodiments, the agent is allowed to circulate for from about 30 seconds to about 30 minutes. In some embodiments, the agent is allowed to circulate for about 3 minutes. In some embodiments, the agent is fixed by immersion fixation. In some embodiments, the agent is fluoro-Ruby-Dextran.

In one aspect, described herein is a method for identifying a modulator of the permeability of the blood-brain barrier during development, the method comprising administering a candidate modulator agent to an embryo injecting the liver of an embryo with a detectable agent while the embryo is connected to the maternal circulation via the umbilical cord, allowing the dye to circulate in the bloodstream, and detecting a signal from the detectable agent in blood vessels within the brain and within brain tissue separated from the bloodstream by the blood-brain barrier, wherein the candidate modulator is determined to increase permeability of the blood-brain barrier if the ratio of signal detected in brain tissue:signal detected in the blood vessels within the brain is lower than a reference level and wherein the candidate modulator is determined to decrease permeability of the blood-brain barrier if the ratio of signal detected in brain tissue:signal detected in the blood vessels within the brain is higher than a reference level. In some embodiments, the agent is a fixable dye. In some embodiments, the total volume of the injection is less than or equal to 1 uL for a murine embryo of about 13.5 days age, less than or equal to 2 uL for a murine embryo of about 14.5 days of age, and less than or equal to 5 uL for a murine embryo of about 15 days of age or older. In some embodiments, the agent is allowed to circulate for from about 30 seconds to about 30 minutes. In some embodiments, the agent is allowed to circulate for about 3 minutes. In some embodiments, the agent is fixed by immersion fixation. In some embodiments, the agent is fluoro-Ruby-Dextran.

### BRIEF DESCRIPTION OF THE DRAWINGS

FIGS. 1A-1C demonstrate an unbiased approach to identify genes involved in BBB formation. Transcriptional profile comparison of vascular cells isolated from forebrain (BBB) with vascular cells isolated from lung (non BBB) at the critical barrier-genesis period (E13.5). FIG. 1A depicts a dot plot representation of Affymetrix GeneChips data. Each point reflects the average expression value of a single probe set on the GeneChip in forebrain (X-axis) and lung (Y-axis). Forebrain expression value of above 500 is presented. Dots between the lines indicate 2 fold or less transcriptional differences between forebrain and lung. Certain of the dots below the lower line indicate 5 fold higher expression value in forebrain then in lung. FIG. 2B depicts a graph demonstrating that endothelium markers are enriched in both forebrain (black bars) and lung (white bars), while neuronal, astrocytic or pericystic markers show low expression values indicating efficient isolation and endothelial enrichment. FIG. 2C depicts a graph demonstrating that genes involved in transport across the barrier have high and differential expression pattern at E13.5 while tight junction markers have either low or non differential expression pattern (forebrain-black bars, lung-white bars). All data represent four biological replicates of 4 different litters. Error bars represent standard deviation.

FIG. 2 demonstrates that Mfsd2a is a candidate gene with highly selective BBB expression pattern. FIG. 2 depicts a graph of microarray analysis demonstrating that Mfsd2a expression is 80 fold higher in BBB vasculature (forebrain-black bar) than in non-BBB vasculature (lung-white bar) at E13.5.

FIGS. 3A-3B demonstrate that Mfsd2a is required for embryonic formation of a functional BBB in vivo. 10 kDa Ruby-Dextran tracer injections of Mfsd2a^{-/-}/wild-type litter mates at E15.5-E16.5 reveal aberrant barrier-genesis in the absence of Mfsd2a. Confocal microscopy revealed that both diffuse tracer and neuro-progenitor cells stained with tracer in Mfsd2a^{-/-} but not in controls. FIG. 3A depicts a graph of the quantification of brain parenchyma cells stained with injected tracer in controls (black bars) and Mfsd2a^{-/-} cortical plates (white bars). Quantification is shown as percentage of samples (sections) that exhibits the indicated number of tracer positive parenchyma cells of total sample. Mfsd2a^{-/-} mice have no detectable angiogenesis defect as demonstrated with epi-fluorescence microscope imaging. PECAM-vascular staining revealed normal gross vessel morphology. FIG. 3B depicts a graph of vascular coverage quantification of E15.5-E16.6 Mfsd2a^{-/-} and wild-type dorsal forebrain cortical plates. No significant difference is found between vascular coverage averages of wild-type (black bar) and Mfsd2a^{-/-} (white bar) samples (asterisk P>0.5). Error bars represent SEM. All results represent quantification of 3 wild-type and 4 KO embryos of 3 different litters (at least 2.0 sections per embryo).

FIGS. 4A-4B demonstrate expression profile comparison of forebrain (BBB) and lung (non BBB) vascular cells. Endothelial cells isolate from the vascular specific Tie2-GFP reporter mouse at E13.5 forebrain and lung are used to compare BBB and non BBB vasculature. FIG. 4A depicts a table of demonstrating that pan-endothelial markers show relative high expression values in both populations. FIG. 4B depicts a table demonstrating that many genes involved in transport across the barrier, known as adult BBB markers, are highly and differentially expressed in brain endothelial cells. Expression values (a.u) are averages of four biological replicates.

FIG. 5 depicts a schematic of a novel tracer injection method which reveals a temporal profile of functional BBB formation in the embryonic cortex. Embryos were exposed via a cesarean incision and a small volume of tracer (1 μl at E13.5; 2 μl at E14.5; 5 μl at E15.5) was injected into the embryonic liver. Fenestrated liver vasculature allowed rapid tracer uptake into the embryonic circulation. Brains were dissected and fixed by immersion in 4% PFA.

FIGS. 6A-6B demonstrate that expression profiling identifies genes involved in BBB formation. FIG. 6A depicts a dot plot representation of Affymetrix GeneChip data showing transcriptional profile of cortical (BBB) and lung (non-BBB) endothelial cells isolated at the critical barrier-genesis period (E13.5). Dots reflect average expression of a probe in the cortex (x-axis) and lung (y-axis). Cortex expression values above 500 arbitrary expression units (a.u) are presented. FIG. 6B depict graphs of barrier-genesis specific transporters, transcription factors, and secreted and transmembrane proteins were significantly enriched in the endothelial cells of the cortex. Data are mean±standard deviation (s.d.) of 4 biological replicates from 4 litters.

FIG. 7 depicts a graph of microarray analysis demonstrating Mfsd2a expression is ˜80-fold higher in the cortex endothelial cells (left bar) than in lung endothelial cells (right bar) at E13.5. Data are mean±standard deviation of 4 biological replicates from 4 litters.

FIGS. 8A-8B demonstrate that Mfsd2a is required for the establishment of a functional BBB but not for CNS angiogenesis in vivo. FIG. 8A depicts a graph of the quantification of tracer-filled parenchyma cells in control versus Mfsd2a−/− cortical plates. FIG. 8B depicts a graph of the quantification of vascular coverage in wild-type and Mfsd2a−/− samples showed no significant difference (P>0.5). Data are mean±S.E.M. n=7 embryos per genotype from 4 litters, 20 sections per embryo. N.S., not significant.

FIGS. 9A-9D demonstrate that Mfsd2a is required specifically to suppress transcytosis in brain endothelium to maintain BBB integreity. FIG. 9A depicts electro micrographs demonstrating that no overt tight junction defect was found in brain endothelial cells from mice lacking Mfsd2a. Left, electron micrographs from wild-type (Mfsd2a+/+) and mutant (Mfsd2a−/−) E17.5 embryos showing no difference in electron-dense tight junction ultrastructure, with typical “kissing points” (small arrows) where plasma membranes from adjacent cells are fused. Right, in HRP-injected P90 adult mice, the electron-dense DAB reaction (black) filled the lumen and diffused in part of junction cleft to stop sharply at the junction without parenchymal leak (arrows). FIG. 9B depicts electron micrographs demonstrating that increased vesicular activity was evidenced by electron microscopic examination of brain endothelial cells in E17.5 embryos lacking Mfsd2a. Left, wild-type endothelial cells displayed only very few vesicles (arrow). Right, Mfsd2a−/− endothelium contained a high number of various types of vesicles, as illustrated for luminal membrane-connected (arrows) and abluminal membrane-connected (arrowheads) vesicles. FIG. 9C depicts quantification of the density of various type of vesicles illustrated in FIG. 9B, including luminal membrane-connected type I and II, cytoplasmic, and abluminal membrane-connected vesicles. Absolute values of vesicular density are shown in left and middle histograms, and expressed as percent of wild-type littermate controls (dotted line) in the right histogram. See Table 1 for detailed analysis. Noteworthy, an almost 3-folds increase was measured for pinocytotic type II luminal vesicles (typical “pinching in” vesicles with a neck-like structure connected to the lumen) in Mfsd2a−/− endothelium. FIG. 9D depicts images demonstrating that increased transcytosis is evident by HRP-filled vesicles traveling from luminal to the ablumenal side in the brain endothelial cells in HRP-injected adult Mfsd2a−/− mice. Left, the P90 HRP-injected wild-type littermates showed HRP activity confined within the lumen with no HRP-filled vesicles. Right, many HRP-filled vesicles were found in Mfsd2a−/− brain endothelial cells. Dye uptake from luminal invaginations (arrows) is followed by dye transport and release to the basement membrane (abluminal) side (*). Ab: abluminal, E: endothelium, L: lumen. Scale bars: a,b: 100 nm; c, 200 nm.

FIG. 10 depicts a diagram illustrating two unique BBB properties of CNS endothelial cells. Compared to the endothelial cells from the rest of the body, CNS endothelial cells which possess a BBB are characterized by (1) highly specialized tight junctions sealing the space between adjacent cells, and (2) unusually low rate of transcytosis for an almost absent vesicular transport from the vessel lumen to the brain parenchyma.

FIGS. 11A-11B demonstrate that pericyte coverage and ultrastructure are normal in Mfsd2a−/− brain. Mfsd2a−/− mice exhibit normal pericyte coverage. Co-staining of endothelium (claudin5) and pericytes (CD13 in FIG. 11A and PDGFRβ in FIG. 11B) revealed no overt difference in pericyte coverage of dorsal cortex vessels between wild-type and Mfsd2a−/− mice at P5. Quantification of vascular coverage in both FIG. 11A and FIG. 11B showed no significant difference between wild-type and Mfsd2a−/− samples (P>0.5). Data are mean±s.e.m. n=3 pups per genotype, 20 sections per embryo. N.S., not significant.

FIG. 12 demonstrates that Mfsd2a gene expression is down regulated in two mouse models with reduced pericyte coverage. Analysis of micro array data from (Armulik, A. et al.) showed high levels of Mfsd2a expression in the adult brain microvasculature but significant decrease in levels of Mfsd2a expression in mice that have reduced pericyte coverage at the BBB. Pdgfbret/ret mice (mouse model 1) where PDGF-B binding to heparan sulphate proteoglycans was disrupted exhibit major loss of pericyte coverage (74% of reduction) 6, also showed a dramatic decrease in Mfsd2a expression (74% of reduction) in the adult brain compared to that of littermate control mice. Similarly, Tie2Cre/R26P+/0, pdgfb−/− mice (mouse model 2) in which Pdgfb null alleles were complemented by one copy of human PDGF-B transgene showed a less dramatic loss of pericyte coverage (60% of reduction) 6 and we found a lesser degree of decrease in Mfsd2a expression (53% of reduction). **(P=0.004), ***(P=1×10-5). Bars reflect normalized signal of the Mfsd2a probe (1428223_at) in adult brain or cortex microvascular fragments (a.u). Data are mean±s.d. of 4 biological replicates.

FIGS. 13A-13B demonstrate the blood-brain barrier-specific expression of the genes described herein.

FIG. 14 depicts graphs demonstrating that barrier-genesis specific transporters, transcription factors, and secreted and transmembrane proteins were significantly enriched in the cortical endothelial cells. All data are mean±s.d. n=4 litters (4 biological replicates).

FIG. 15 depicts a graph depicting spectrophotometric quantification of 10-kDa dextran-tracer from cortical extracts of P90 mice, 16 h post intravenous injection, indicating that BBB leakiness in Mfsd2a^{-/-} mice persists into adulthood (N=3 mice per genotype).

FIG. 16 depicts graphs demonstrating that Mfsd2a^{-/-} mice exhibit normal vascular patterning. No abnormalities were found in cortical vascular density branching and capillary diameter. Quantification of wild-type and Mfsd2a^{-/-} samples (n=4 embryos per genotype). All data are mean+s.e.m. MUT, mutant; N.S. not significant; WT, wild type. P=0.05 (Mann-Whitney U-test).

FIGS. 17A-17C demonstrate that perinatal and adult mice lacking Mfsd2a doe not display changes in cerebrovascular network properties or signs of vascular degeneration. FIG. 17A depicts graphs demonstrating that no abnormalities were found in cortical capillary density and vascular branching (top panels) as well as capillary diameter (bottom panels) of postnatal (P4, left) and adult (P70, right) Mfsd2a^{-/-} mice. Data are mean±s.e.m. n=3 animals per genotype, 20 sections per animal. FIG. 17B depicts a graph demonstrating that no abnormalities in arterial distribution and specification in Mfsd2a^{-/-} were found. Data are mean±s.e.m. n=3 animals per genotype, 20 sections per animal. FIG. 17C depicts images of electron-microscopy examination of older Mfsd2a−/− mice which did not reveal signs of cerebrovascular degeneration. Left, the overall capillary structure (for example, cell size, shape of the nucleus, thickness of the vessel wall, basement membrane integrity and pericyte attachment) did not differ between wild-type and mutant mice. Right, at higher magnification, normal features, such as pericyte (asterisk) attachment within a normal basement membrane (between arrows), could be observed in mice lacking Mfsd2a.

FIGS. 18A-18C demonstrate that pericyte coverage, attachment and ultrastructure are normal in Mfsd2a^{-/-} mice. FIGS. 18A-18B demonstrate that Mfsd2a^{-/-} mice exhibit normal pericyte coverage. Co-staining of endothelium and pericytes (CD13 in FIG. 18A and Pdgfrβ in FIG. 18B) revealed no overt difference in pericyte coverage of dorsal cortex vessels between wild-type and Mfsd2a^{-/-} mice at P5. Quantification of vascular coverage in both showed no significant difference between wild-type and Mfsd2a−^{/-} samples (P>0.5). Data are mean±s.e.m. n=3 pups per genotype, 20 sections per animal. FIG. 18C depicts electron micrographs of longitudinal capillary sections which revealed that pericytes had normal appearance and were well positioned on the vessel walls in Mfsd2a^{-/-} adult mice; pericytes were adjacent to endothelial cells and shared a common basement membrane. L, lumen; P, pericyte.

FIGS. 19A-19B demonstrate that gene expression and Mfsd2a protein levels are downregulated in mouse models of reduced pericyte coverage. FIG. 19A depicts a graph of analysis of microarray data⁵ which demonstrates high levels of Msd2a expression in wild-type adult brain microvasculature, but a significant decrease in levels of Mfsd2a expression in mice that have reduced pericyte coverage at the BBB. Pdgfbr et/ret mice (mouse model 1), where Pdgfβ binding to heparan sulphate proteoglycans was disrupted, exhibited a major loss of pericyte coverage (74 reduction)⁵ and showed a dramatic decrease in Mfsd2a expression (74 reduction) compared to that of littermate controls. Similarly, Tie2^{cre}/R26^{P0}/Pdgfb^{-/-} mice (mouse model 2) in which Pdgfb-null alleles were complemented by one copy of human PDGFB transgene showed a less dramatic loss of pericyte coverage (60 reduction)⁵ and a smaller decrease in Mfsd2a expression (53 reduction). P=0.004, P=1×10-5). Bars reflect normalized signal of the Mftd2a probe (1428223_at) in adult brain or cortex microvascular fragments (a.u.). Data are mean±s.d. of 4 biological replicates. FIG. 19B depicts quantification of mean fluorescence intensity per vascular profile, demonstrating significant reduction of Mfsd2a signal in Pdgfbre/ret capillaries compared to controls. Data are mean±s.e.m. n=2 animals per genotype, 60 images quantified of at least 600 vascular profiles per animal.

FIGS. 20A-20B demonstrate that immuno-electron-microscopy reveals the subcellular localization of Mfsd2a on the plasma membrane and vesicles, but not in tight junctions of cerebral endothelial cells. FIG. 20A depicts electron micrographs showing silver-enhanced immunogold labelling of Mfsd2a in cerebral cortex capillaries from wild-type (left) but not in Mfsd2a^{-/-} mice (right), demonstrating staining specificity. In FIG. 20B, the top panels depict three representative examples of Mfsd2a localization on the plasma membrane (arrows) and in the cytoplasm (arrowheads), but not in tight junctions (asterisk). Bottom panels depict high magnification representative examples of Mfsd2a localization on the luminal plasma membrane (arrows), associated with luminal invaginating vesicles (iv v) and with cytoplasmic vesicles (arrowheads). All samples are of cortical vessels from adult mice (P30-P90). n=2 for each genotype. L, lumen.

FIG. 21 depicts a graph demonstrating that Mfsd2a^{-/-} BBB is more permeable to an antibody. Experiment was conducted as described for FIG. 15, using IgG-Cy3 (Goat anti-human IgG antibody) instead of a dextran construct.

### DETAILED DESCRIPTION

As described herein, the inventors have discovered that certain genes, e.g., Mfsd2A, are necessary for the formation of the blood brain barrier (BBB), but not for angiogenesis. Accordingly, modulating the level and/or activity of these BBB key regulatory genes can therefore affect the formation and/or integrity (i.e. the permeability) of the blood brain barrier without disadvantageous side effects on other structures or processes. Provided herein are methods of modulating the permeability of the blood brain barrier by modulating the level and/or activity of one or more of these BBB key regulatory genes.

A blood-brain barrier (or BBB) is the structure that separates circulating blood from the central nervous system (CNS). The BBB lines the capillaries associated with the CNS and is comprised of endothelial cells and the tight junctions between them. The BBB also includes a basement membrane and astrocytic endfeet. The BBB generally excludes large hydrophilic molecules and bacteria from entering the CNS while allowing the passage of small hydrophobic molecules such as oxygen. Certain molecules are actively transported across the BBB, e.g. glucose.

While the BBB is generally very effective at excluding, e.g. bacterial pathogens, from the CNS, when medical practitioners wish to deliver a drug to the CNS, the BBB poses a formidable obstacle. For example, antibodies and most antibiotics will not cross the BBB. The degradation of the BBB is a feature of many neurodegenerative diseases, e.g. multiple sclerosis. Accordingly, methods for modulating the permeability of the BBB, both by increasing or decreasing the permeability, have a role in the treatment of a wide variety of diseases that impact the CNS.

As described herein, the inventors have identified certain genes which are expressed during BBB formation. The identified genes are sometimes referred to herein as BBB key regulatory genes to indicate their relation to being a gene which is integral for the formation and/or location of the BBB. In some embodiments, the BBB key regulatory gene can be a marker for the location, presence, and/or differentiation of the BBB. In some embodiments, the BBB key regulatory gene can be a target (e.g. a therapeutic target), e.g. to modulate the BBB in accordance with the methods described herein.

In one aspect, described herein is a method of modulating the permeability of the blood-brain barrier in a subject, the method comprising administering an inhibitor of a BBB key regulatory gene, e.g., Mfsd2A to the subject, whereby the permeability of the blood-brain barrier is increased; or administering an agonist of a BBB key regulatory gene, e.g., Mfsd2A to the subject, whereby the permeability of the blood-brain barrier is decreased. In one aspect, described herein is a method of treatment, the method comprising administering an inhibitor of a BBB key regulatory gene, e.g., Mfsd2A to a subject in need of increased permeability of the blood-brain barrier or administering an agonist of a BBB key regulatory gene, e.g., Mfsd2A to the subject in need of decreased permeability of the blood-brain barrier.

Exemplary BBB key regulatory genes are listed in Table 2. In some embodiments, the BBB key regulatory gene which is modulated by the administration of an agonist or inhibitor is selected from one of the genes of Table 2. The gene names listed in Table 2 are common names.

TABLE 2

| Name | NCBI GENE ID |
|---|---|
| Mfsd2A | 84879 |
| Slco1C1 | 53919 |
| Slc38A5 | 92745 |
| LRP8 | 7804 |
| Slc3A2 | 6520 |
| Slc7A5 | 8140 |
| Slc6A6 | 6533 |
| Igfbp7 | 3490 |
| Glut1 | 6513 |
| Slc40A1 | 30061 |
| Slc30A1 | 7779 |

In some embodiments, the BBB key regulatory gene is Mfsd2A. As described herein, “Mfsd2A” or “major facilitator superfamily domain-containing 2A” refers to a transmembrane protein believed to mediate the uptake and transport of tunicamycin. Mfsd2A has a 12 transmembrane alpha-helical domain structure with similarity to the bacterial Na+/melibiose symporters. The sequences of Mfsd2A polypeptides and nucleic acids encoding such polypeptides are known in the art for a number of species, e.g. human Mfsd2A (NCBI Gene ID: 84879 (polypeptide; NCBI Ref Seq: NP_001129965; SEQ ID NO: 1 or 3)(mRNA; NCBI Ref Seq: NM_001136493; SEQ ID NO:2)

A Mfsd2A polypeptide can comprise SEQ ID NO: 1 or 3 or a homolog, variant, and/or functional fragment thereof. A nucleic acid encoding a Mfsd2A polypeptide can comprise SEQ ID NO: 2 or a homolog or variant thereof. The polypeptide sequences and nucleic acid sequences encoding any of the other BBB key regulatory genes described herein can readily by obtained by searching the “Gene” Database of the NCBI (available on the World Wide Web at http://www.ncbi.nlm.nih.gov/) using the common name or NCBI Gene ID number as the query and selecting the first returned Homo sapiens gene.

As used herein, a “functional fragment” of, e.g. SEQ ID NO: 1 or 3, is a fragment or segment of that polypeptide which can promote formation of the BBB at least 10% as strongly as the reference polypeptide (i.e. SEQ ID NO: 1 or 3), e.g. at least 10%, at least 20%, at least 30%, at least 40%, at least 50%, at least 75%, at least 90%, at least 100% as strongly, or more strongly. Assays for measuring the formation of the BBB are known in the art and described herein, e.g., by way of non-limiting example, the migration of tracer dyes out of vessels in the brain using the embryonic models described in the Examples herein can be used to quantitate the formation and/or integrity of the BBB. A functional fragment can comprise conservative substitutions of the sequences disclosed herein.

Variants of the polypeptides described herein (e.g. SEQ ID NO: 1 or 3) can be obtained by mutations of native nucleotide or amino acid sequences, for example SEQ ID NO: 1 or 3 or a nucleotide sequence encoding a peptide comprising SEQ ID NO:1 or 3. A “variant,” as referred to herein, is a polypeptide substantially homologous to a native polypeptide described herein (e.g. SEQ ID NO: 1 or 3), but which has an amino acid sequence different from that of one of the sequences described herein because of one or a plurality of deletions, insertions or substitutions.

The variant amino acid or DNA sequence preferably is at least 60%, at least 70%, at least 80%, at least 90%, at least 91%, at least 92%, at least 93%, at least 94%, at least 95%, at least 96%, at least 97%, at least 98%, at least 99%, or more, identical to the sequence from which it is derived (referred to herein as an “original” sequence). The degree of homology (percent identity) between an original and a mutant sequence can be determined, for example, by comparing the two sequences using freely available computer programs commonly employed for this purpose on the world wide web. The variant amino acid or DNA sequence preferably is at least 60%, at least 70%, at least 80%, at least 90%, at least 91%, at least 92%, at least 93%, at least 94%, at least 95%, at least 96%, at least 97%, at least 98%, at least 99%, or more, similar to the sequence from which it is derived (referred to herein as an “original” sequence). The degree of similarity (percent similarity) between an original and a mutant sequence can be determined, for example, by using a similarity matrix. Similarity matrices are well known in the art and a number of tools for comparing two sequences using similarity matrices are freely available online, e.g. BLASTp (available on the world wide web at http://blast.ncbi.nlm.nih.gov).

Alterations of the original amino acid sequence can be accomplished by any of a number of known techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. Techniques for making such alterations include those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties. In some embodiments, an isolated peptide as described herein can be chemically synthesized and mutations can be incorporated as part of the chemical synthesis process.

Variants can comprise conservatively substituted sequences, meaning that one or more amino acid residues of an original peptide are replaced by different residues, and that the conservatively substituted peptide retains a desired biological activity, i.e., the ability to bind heme, that is essentially equivalent to that of the original peptide. Examples of conservative substitutions include substitutions that do not change the overall or local hydrophobic character, substitutions that do not change the overall or local charge, substitutions by residues of equivalent sidechain size, or substitutions by sidechains with similar reactive groups.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics or substitutions of residues with similar sidechain volume are well known. Isolated peptides comprising conservative amino acid substitutions can be tested in any one of the assays described herein to confirm that a desired activity, e.g. the ability to bind heme, is retained, as determined by the assays described elsewhere herein.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile, Phe, Trp; (2) neutral hydrophilic: Cys, Ser, Thr, Asn, Gln, Ala, Tyr, His, Pro, Gly; (3) acidic: Asp, Glu; (4) basic: His, Lys, Arg; (5) residues that influence chain orientation: Gly, Pro; (6) aromatic: Trp, Tyr, Phe, Pro, His, or hydroxyproline. Non-conservative substitutions will entail exchanging a member of one of these classes for another class.

Particularly preferred conservative substitutions for use in the variants described herein are as follows: Ala into Gly or into Ser; Arg into Lys; Asn into Gln or into His; Asp into Glu or into Asn; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr or into Phe; Tyr into Phe or into Trp; and/or Phe into Val, into Tyr, into Ile or into Leu. In general, conservative substitutions encompass residue exchanges with those of similar physicochemical properties (i.e. substitution of a hydrophobic residue for another hydrophobic amino acid).

Any cysteine residue not involved in maintaining the proper conformation of the isolated peptide as described herein can also be substituted, generally with serine, to improve the oxidative stability of the molecule and prevent aberrant crosslinking. Conversely, cysteine bond(s) can be added to the isolated peptide as described herein to improve its stability or facilitate multimerization.

As described herein, an “inhibitor” of a given BBB key regulatory gene, e.g. an inhibitor of Mfsd2A, refers to an agent which can decrease the expression and/or activity of the targeted expression product (e.g. mRNA encoding the target or a target polypeptide), e.g. by at least 10% or more, e.g. by 10% or more, 50% or more, 70% or more, 80% or more, 90% or more, 95% or more, or 98% or more. The efficacy of an inhibitor of, for example, Mfsd2A, e.g. its ability to decrease the level and/or activity of Mfsd2A can be determined, e.g. by measuring the level of an expression product of Mfsd2A and/or the activity of Mfsd2A (e.g. the permeability of the BBB, the measurement of which is described elsewhere herein). Methods for measuring the level of a given mRNA and/or polypeptide are known to one of skill in the art, e.g. RTPCR with primers can be used to determine the level of RNA and Western blotting with an antibody (e.g. an anti-Mfsd2A antibody, e.g. Cat No. ab105399; Abcam; Cambridge, Mass.) can be used to determine the level of a polypeptide. The activity of, e.g., Mfsd2A can be determined using methods known in the art and described above herein. In some embodiments, the inhibitor of a BBB key regulatory gene can be an inhibitory nucleic acid; an aptamer; an antibody reagent; an antibody; or a small molecule. Non-limiting examples of inhibitors of Mfsd2A can include tunicamycin; tunicamycin analogs; inhibitory anti-Mfsd2A antibodies; inhibitory and nucleic acids.

In some embodiments, the compounds of the invention have a structural formula I:

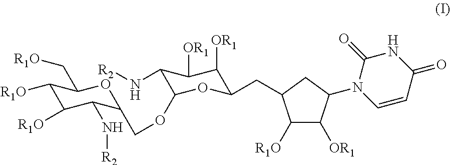

wherein:

each occurrence of R₁ and R₂ is independently hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —C(═O)R^{B}; —CO₂R^{B}; −; —CN; —SCN; —SR^{B}; —SOR^{B}; —SO₂R^{B}; —NO₂; —N(R^{B})₂; —NHC(O)R^{B}; or —C(R^{B})₃; wherein each occurrence of R^{B} is independently hydrogen; halogen; a protecting group; aliphatic; heteroaliphatic; acyl; aryl moiety; heteroaryl; hydroxyl; aloxy; aryloxy; alkylthioxy; arylthioxy; amino; alkylamino; dialkylamino; heteroaryloxy; heteroarylthioxy; or alkylhalo.

In some embodiments, R₁ is hydrogen. In some embodiments, at least one R₁ is hydrogen. In some embodiments all R₁ are hydrogen.

In some embodiments, R₁ is a straight chain aliphatic. In some embodiments, R₁ is a branched chain aliphatic. In some embodiments, R₁ is a straight chain heteroaliphatic. In some embodiments, R₁ is a branched chain heteroaliphatic.

In some embodiments, R₁ is C₁₋₄ alkyl, C₂₋₄ alkenyl, or C₂₋₄ alkynyl. In some embodiments, R₁ is aryl or heteroaryl. In some embodiments R₁ is acyl.

In some embodiments, R₁ is C₁₋₄ alkyl. In some embodiments, R₁ is methyl, ethyl, n-propyl, i-propyl, n-butyl, i-butyl, or t-butyl.

In some embodiments, R₁ is —C(O)CH₃. In some embodiments, at least one R₁ is —C(O)CH₃. In some embodiments all R₁ are —C(O)CH₃.

In some embodiments, R₁ is a protecting group.

In some embodiments, R₁ is optionally substituted aryl. In some embodiments, R₁ is optionally substituted heteroaryl.

In some embodiments, R₂ is hydrogen. In some embodiments, at least one R₂ is hydrogen. In some embodiments all R₂ are hydrogen.

In some embodiments, R₂ is a straight chain aliphatic. In some embodiments, R₂ is a branched chain aliphatic. In some embodiments, R₂ is a straight chain heteroaliphatic. In some embodiments, R₂ is a branched chain heteroaliphatic.

In some embodiments, R₂ is C₁₋₄ alkyl, C₂₋₄ alkenyl, or C₂₋₄ alkynyl. In some embodiments, R₂ is aryl or heteroaryl. In some embodiments R₂ is acyl.

In some embodiments, R₂ is C₁₋₄ alkyl. In some embodiments, R₂ is methyl, ethyl, n-propyl, i-propyl, n-butyl, i-butyl, or t-butyl.

In some embodiments, R₂ is —C(O)CH₃. In some embodiments, at least one R₂ is —C(O)CH₃. In some embodiments all R₂ are —C(O)CH₃.

In some embodiments, R₂ is a protecting group.

In some embodiments, R₂ is optionally substituted aryl. In some embodiments, R₁ is optionally substituted heteroaryl.

In some embodiments, R₂ is

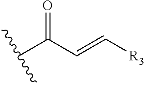

wherein R₃ is cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroary.

In some embodiments, R₃ is C₁₋₄ alkyl, C₂₋₄ alkenyl, or C₂₋₄ alkynyl. In some embodiments, R₃ is aryl or heteroaryl.

In some embodiments, R₃ is C₁₋₄ alkyl. In some embodiments, R₂ is methyl, ethyl, n-propyl, i-propyl, n-butyl, i-butyl, or t-butyl. In some embodiments, R₃ is i-propyl.

In some embodiments, formula I is tunicamycin:

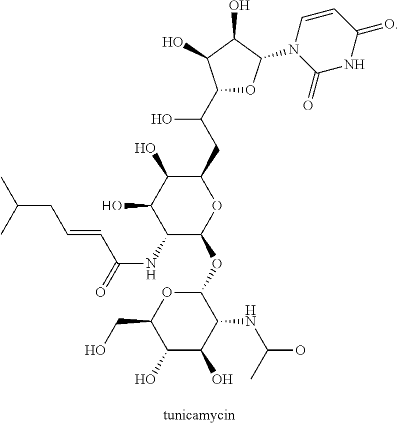 [SMILES: CC(C)C/C=C/C(=O)N[C@H]1[C@H](O[C@H]2O[C@H](CO)[C@@H](O)[C@H](O)[C@H]2NC(C)O)O[C@H](CC(O)[C@H]2O[C@@H](n3ccc(=O)[nH]c3=O)[C@H](O)[C@@H]2O)[C@H](O)[C@@H]1O]

A “protecting group” is introduced into a molecule by chemical modification of a functional group. Protecting groups can be but are not limited to alcohol protecting groups (e.g., ester protection, ether protection, ether silyl protection), amine protecting groups (e g, amine protection, amide protection, carbamate protection, sulfonamide protection), carbonyl protecting groups (e.g., acetal protection, dithiane/dithiolane protection), carboxylic acid protecting groups (e.g., ester protection, ester silyl protection, orthoester protection, oxazoline protection). Examples of ester protection: acetoxy (Ac) and pivolyl (Piv) groups. Examples of ether protections: methyl (Me), methoxymethyl (MOM), methylethoxymethyl (MEM), tetrahydropyranyl (THP), benzyl (Bn), p-methoxybenzyl (PMB), and trityl or tiphenylmethane (Tr) groups. Examples of ether sily protection: trimethylsilyl (TMS), triispropylsilyl (TIPS), tert-butyldimethylsilyl (TBS or TBDMS) and [2-(trimethylsilyl)ethoxy]methyl (SEM) groups. Examples of amine protection: benzyl (Bn) and p-methoxyphenyl (PMP) groups. Examples of amide protection: acetyl (Ac), trifluororacetyl (TFA) and Trichloroacetyl groups. Examples of carbamate protection: tert-butyloxycarbonyl (BOC), carbobenzyloxy (Cbz or Z), vinyloxycarbonyl (Voc), allyloxycarbonyl (Alloc), 9-fluorenylmethyloxycarbonyl (Fmoc) groups. Examples of sulfonamide protection: tosyl (Ts) and nosyl (Ns) groups. Examples of acetal protection: dimethyl acetal, 1,3-dioxolanes, 1,3-dioxane. Examples of dithiane/dithiolane protection: 1,3-dithiane, 1,3-dithiolane. One of ordinary skill in the art would know to refer for example to “Protective Groups in Organic Synthesis” Wuts P.G.M. and Greene T. W., editions Wiley-Interscience 4 the Edition (Oct. 30, 2006) which is incorporated in entirety by reference.

As used herein, the terms “alkyl,” “alkenyl” and the prefix “alk-” are inclusive of both straight chain and branched chain groups and of cyclic groups, i.e. cycloalkyl and cycloalkenyl. Unless otherwise specified, these groups contain from 1 to 20 carbon atoms, with alkenyl groups containing from 2 to 20 carbon atoms. Preferred groups have a total of up to 10 carbon atoms. Cyclic groups can be monocyclic or polycyclic and preferably have from 3 to 10 ring carbon atoms. Exemplary cyclic groups include cyclopropyl, cyclopentyl, cyclohexyl, cyclopropylmethyl, adamantly, norbornane, and norbornene. This is also true of groups that include the prefix “alkyl-,” such as alkylcarboxylic acid, alkyl alcohol, alkylcarboxylate, alkylaryl, and the like. Examples of suitable alkylcarboxylic acid groups are methylcarboxylic acid, ethylcarboxylic acid, and the like. Examples of suitable alkylacohols are methylalcohol, ethylalcohol, isopropylalcohol, 2-methylpropan-1-ol, and the like. Examples of suitable alkylcarboxylates are methylcarboxylate, ethylcarboxylate, and the like. Examples of suitable alkyl aryl groups are benzyl, phenylpropyl, and the like.

These may be straight chain or branched, saturated or unsaturated aliphatic hydrocarbon, which may be optionally inserted with N, O, or S. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like.

As used herein, the term “alkenyl” means an alkyl, as defined above, containing at least one double bond between adjacent carbon atoms. Alkenyls include both cis and trans isomers. Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like.

As used herein, the term “alkynyl” means any alkyl or alkenyl, as defined above, which additionally contains at least one triple bond between adjacent carbons. Representative straight chain and branched alkynyls include acetylenyl, propynyl, 1-butynyl, 2-butynyl, 1-pentynyl, 2-pentynyl, 3-methyl-1 butynyl, and the like.

The term “aryl” as used herein includes carbocyclic aromatic rings or ring systems. As used herein, the term “aryl” refers to an aromatic 5-8 membered monocyclic, 8-12 membered bicyclic, or 11-14 membered tricyclic ring system. Examples of aryl groups include phenyl, naphthyl, biphenyl, fluorenyl and indenyl.

The term “heteroaryl” includes aromatic rings or ring systems that contain at least one ring hetero atom (e.g., O, S, N). As used herein, the term “heteroaryl” refers to an aromatic 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2, 3, or 4 atoms of each ring may be substituted by a substituent. Examples of heteroaryl groups include pyridyl, furyl or furanyl, imidazolyl, thiazolyl, benzimidazolyl, pyrimidinyl, thiophenyl or thienyl, quinolinyl, indolyl, oxazolyl, isoquinolinyl, isoindolyl, thiazolyl, pyrrolyl, tetrazolyl, pyrazolyl, oxazolyl, thiazolyl, benzofuranyl, benzothiophenyl, carbazolyl, benzoxazolyl, benzimidazolyl, quinoxalinyl, benzothiazolyl, naphthyridinyl, isoxazolyl, isothiazolyl, purinyl, quinazolinyl, and the like.

Heteroaryl rings may also be fused with one or more cyclic hydrocarbon, heterocyclic, aryl, or heteroaryl rings. Heteroaryl includes, but is not limited to, 5-membered heteroaryls having one hetero atom (e.g., thiophenes, pyrroles, furans); 5-membered heteroaryls having two heteroatoms in 1,2 or 1,3 positions (e.g., oxazoles, pyrazoles, imidazoles, thiazoles, purines); 5-membered heteroaryls having three heteroatoms (e.g., triazoles, thiadiazoles); 5-membered heteroaryls having 3 heteroatoms; 6-membered heteroaryls with one heteroatom (e.g., pyridine, quinoline, isoquinoline, phenanthrine, 5,6-cycloheptenopyridine); 6-membered heteroaryls with two heteroatoms (e.g., pyridazines, cinnolines, phthalazines, pyrazines, pyrimidines, quinazolines); 6-membered heretoaryls with three heteroatoms (e.g., 1,3,5-triazine); and 6-membered heteroaryls with four heteroatoms. Particularly preferred heteroaryl groups are 5-10-membered rings with 1-3 heteroatoms selected from O, S, and N.

The aryl, and heteroaryl groups can be unsubstituted or substituted by one or more substituents independently selected from the group consisting of alkyl, alkoxy, methylenedioxy, ethylenedioxy, alkylthio, haloalkyl, haoalkoxy, haloalkylthio, halogen, nitro, hydroxy, mercapto, cyano, carboxy, formyl, aryl, aryloxy, arylthio, arylalkoxy, arylalkylthio, heteroaryl, heteroaryloxy, heteroarylalkoxy, heteroarylalkylthio, amino, alkylamino, dialkylamino, heterocyclyl, heterocycloalkyl, alkylcarbonyl, alkenylcarbonyl, alkoxycarbonyl, haloalkylcarbonyl, haloalkoxycarbonyl, alkylthiocarbonyl, arylcarbonyl, heteroarylcarbonyl, aryloxycarbonyl, heteroaryloxycarbonyl, arylthiocarbonyl, heteroarylthiocarbonyl, alkanoyloxy, alkanoylthio, alkanoylamino, arylcarbonyloxy, arylcarbonythio, alkylaminosulfonyl, alkylsulfonyl, arylsulfonyl, heteroarylsulfonyl, aryldiazinyl, alkylsulfonylamino, arylsulfonylamino, arylalkylsulfonylamino, alkylcarbonylamino, alkenylcarbonylamino, arylcarbonylamino, arylalkylcarbonylamino, arylcarbonylaminoalkyl, heteroarylcarbonylamino, heteroarylalkycarbonylamino, alkylsulfonylamino, alkenylsulfonylamino, arylsulfonylamino, arylalkylsulfonylamino, heteroarylsulfonylamino, heteroarylalkylsulfonylamino, alkylaminocarbonylamino, alkenylaminocarbonylamino, arylaminocarbonylamino, arylalkylaminocarbonylamino, heteroarylaminocarbonylamino, heteroarylalkylaminocarbonylamino and, in the case of heterocyclyl, oxo. If other groups are described as being “substituted” or “optionally substituted,” then those groups can also be substituted by one or more of the above enumerated substituents.

The term “arylalkyl,” as used herein, refers to a group comprising an aryl group attached to the parent molecular moiety through an alkyl group.

As used herein, the term “cyclyl” refers to a nonaromatic 5-8 membered monocyclic, 8-12 membered bicyclic, or 11-14 membered tricyclic ring system, which can be saturated or partially unsaturated. Representative saturated cyclyl groups include, but are not limited to, cyclopropyl, cyclobutyl, cyclopentyl, cyclopentenyl, cyclohexyl, cyclohexenyl, cycloheptyl, cyclooctyl, and the like; while unsaturated cyclyl groups include cyclopentenyl and cyclohexenyl, and the like.

The terms “heterocycle”, “heterocyclyl” and “heterocyclic group” are recognized in the art and refer to nonaromatic 3- to about 14-membered ring structures, such as 3- to about 7-membered rings, whose ring structures include one to four heteroatoms, 5-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively), wherein 0, 1, 2 or 3 atoms of each ring may be substituted by a substituent. The heterocycle may include portions which are saturated or unsaturated. In some embodiments, the heterocycle may include two or more rings (e.g., cycloalkyls, cycloalkenyls, cycloalkynyls, aryls and/or heterocyclyls) in which two or more carbons are common to two adjoining rings, e.g., the rings are “fused rings.” In some embodiments, the heterocycle may be a “bridged” ring, where rings are joined through non-adjacent atoms, e.g., three or more atoms are common to both rings. Each of the rings of the heterocycle may be optionally substituted. Examples of heterocyclyl groups include, for example, thiophene, thianthrene, furan, pyran, isobenzofuran, chromene, xanthene, phenoxathin, pyrrole, imidazole, pyrazole, isothiazole, isoxazole, pyridine, pyrazine, pyrimidine, pyridazine, indolizine, isoindole, indole, indazole, purine, quinolizine, isoquinoline, quinoline, phthalazine, naphthyridine, quinoxaline, quinazoline, cinnoline, pteridine, carbazole, carboline, phenanthridine, acridine, pyrimidine, phenanthroline, phenazine, phenarsazine, phenothiazine, furazan, dioxane, morpholine, tetrahydrofurane, phenoxazine, pyrrolidine, oxolane, thiolane, oxazole, piperidine, piperazine, morpholine, lactones, lactams such as azetidinones and pyrrolidinones, sultams, sultones, and the like. The heterocyclic ring may be substituted at one or more positions with substituents including, for example, halogen, aryl, heteroaryl, alkyl, heteroalkyl, aralkyl, alkenyl, alkynyl, cycloalkyl, hydroxyl, amino, nitro, sulfhydryl, imino, amido, phosphonate, phosphinate, carbonyl, carboxyl, silyl, ether, alkylthio, sulfonyl, ketone, aldehyde, ester, a heterocyclyl, an aromatic or heteroaromatic moiety, CF₃, CN, or the like.

As used herein, the term “halogen” refers to iodine, bromine, chlorine, and fluorine.

As used herein, the terms “optionally substituted alkyl,” “optionally substituted cyclyl,” “optionally substituted heterocyclyl,” “optionally substituted aryl,” and “optionally substituted heteroaryl” means that, when substituted, at least one hydrogen atom in said alkyl, cyclyl, heterocylcyl, aryl, or heteroaryl is replaced with a substituent. In the case of an oxo substituent (═O) two hydrogen atoms are replaced. In this regard, substituents include oxo, halogen, alkyl, cyclyl, heterocyclyl, aryl, heteroaryl, —CN, —OR^{x}, —NR^{x}R^{y}, —NR^{x}C(═O)R^{y}, —NR^{x}SO₂R^{y}, —C(═O)R^{x}, —C(═O)OR^{x}, —C(═O)NR^{x}R^{y}, —SOₙR^{x} and —SOₙNR^{x}R^{y}, wherein n is 0, 1 or 2, R^{x} and R^{y} are the same or different and independently hydrogen, alkyl, cyclyl, heterocyclyl, aryl or heterocycle, and each of said alkyl, cyclyl, heterocyclyl, aryl and heterocycle substituents may be further substituted with one or more of oxo, halogen, —OH, —CN, alkyl, —OR^{x}, heterocycle, —NR^{x}R^{y}, —NR^{x}C(═O)R^{y}, —NR^{x}SO₂R^{y}, —C(═O)R^{x}, —C(═O)OR^{x}, —C(═O)NR^{x}R^{y}, —SOₙR^{x} and —SOₙNR^{x}R^{y}.

The term “carbonyl,” as used herein, refers to “C(═O)”.

The terms “acyl,” “carboxyl group,” or “carbonyl group” are recognized in the art and can include such moieties as can be represented by the general formula:

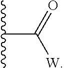

wherein W is OR^{w}, N(R^{w})₂, SR^{w}, or R^{w}, R^{w} being hydrogen, alkyl, heteroalkyl, alkenyl, heteroalkenyl, alkynyl, heteroalkynyl, aryl, heteroaryl, heterocycle, substituted derivatives thereof, or a salt thereof. For example, when W is O-alkyl, the formula represents an “ester,” and when W is OH, the formula represents a “carboxylic acid.” When W is alkyl, the formula represents a “ketone” group, and when W is hydrogen, the formula represents an “aldehyde” group. Those of ordinary skill in the art will understand the use of such terms.

As used herein, the term “substituted” is contemplated to include all permissible substituents of organic compounds, “permissible” being in the context of the chemical rules of valence known to those of ordinary skill in the art. In some cases, “substituted” may generally refer to replacement of a hydrogen with a substituent as described herein. However, “substituted,” as used herein, does not encompass replacement and/or alteration of a key functional group by which a molecule is identified, e.g., such that the “substituted” functional group becomes, through substitution, a different functional group. For example, a “substituted phenyl” must still comprise the phenyl moiety and cannot be modified by substitution, in this definition, to become, e.g., a heteroaryl group such as pyridine. In a broad aspect, the permissible substituents include acyclic and cyclic, branched and unbranched, carbocyclic and heterocyclic, aromatic and nonaromatic, fused, and bridged substituents of organic compounds. Illustrative substituents include, for example, those described herein. The permissible substituents can be one or more and the same or different for appropriate organic compounds. For purposes of this invention, the heteroatoms such as nitrogen may have hydrogen substituents and/or any permissible substituents of organic compounds described herein which satisfy the valencies of the heteroatoms. This invention is not intended to be limited in any manner by the permissible substituents of organic compounds.

As used herein, the term “agonist” of a BBB key regulatory gene refers to any agent that increases the level and/or activity of the BBB key regulatory gene. As used herein, the term “agonist” refers to an agent which increases the expression and/or activity of the target by at least 10% or more, e.g. by 10% or more, 50% or more, 100% or more, 200% or more, 500% or more, or 1000% or more. Non-limiting examples of agonists of a BBB key regulatory gene can include a BBB key regulatory gene polypeptide and a nucleic acid encoding a BBB key regulatory gene polypeptide, e.g. a polypeptide comprising the sequence SEQ ID NO: 1 or 3 or a nucleic acid comprising the sequence of SEQ ID NO: 2 or variants thereof.

In some embodiments, an inhibitor of a BBB key regulatory gene, e.g. an inhibitor of Mfsd2A can be administered to a subject in need of delivery of a CNS therapeutic agent to the central nervous system. The CNS therapeutic agent can be any agent for the treatment of any disease, provided that it is desired that the CNS therapeutic agent reaches the central nervous system. In some embodiments, methods which comprise administering an inhibitor of a BBB key regulatory gene, e.g., an inhibitor of Mfsd2A, to a subject can further comprise administering a CNS therapeutic agent to the subject. Non-limiting examples of such CNS therapeutic agents can include, antibiotics, antibodies, gabapentin, chemotherapeutics, anti-inflammatories, neurotransmitters, morphines, peptides, polypeptides, nucleic acids (e.g. RNAi-based therapies), psychiatric dugs, and/or therapeutic agents for the treatment of brain cancer; encephalitis; hydrocephalus; Parksinson's disease; neuropathic pain; and a condition treated by the administration of psychiatric drugs. The identity of such CNS therapeutic agents are known in the art and described, e.g. in Ghose et al. J Comb Chem 1999 1:55-68 and Pardridge. NeuroRx 2005 2:3-14; each of which is incorporated by reference herein in its entirety. In some embodiments, a central nervous system therapeutic agent can inhibit the activity and/or expression of a therapeutic target gene associated with a central nervous system disease (e.g. examples of such genes are described below herein), e.g. it can be an inhibitory nucleic acid or an inhibitory antibody reagent.

In some embodiments, the central nervous system therapeutic reagent is less than about 500 kDa in size. In some embodiments, the central nervous system therapeutic reagent is less than 500 kDa in size. In some embodiments, the central nervous system therapeutic reagent is less than about 300 kDa in size. In some embodiments, the central nervous system therapeutic reagent is less than 300 kDa in size. In some embodiments, the central nervous system therapeutic reagent is less than about 200 kDa in size. In some embodiments, the central nervous system therapeutic reagent is less than 200 kDa in size. In some embodiments, the central nervous system therapeutic reagent is less than about 70 kDa in size. In some embodiments, the central nervous system therapeutic reagent is less than 70 kDa in size. In some embodiments, the central nervous system therapeutic reagent can be, e.g. an enzyme, an antibody reagent, a sugar, and/or a small molecule.

In some embodiments, the CNS therapeutic agent is an agent that does not normally cross the BBB. In some embodiments, the CNS therapeutic agent is an agent that inefficiently crosses the BBB, e.g. a therapeutically effective dose of the agent is unable to cross the BBB when administered systemically. In some embodiments, the CNS therapeutic agent is an agent that does efficiently cross the BBB, e.g. a therapeutically effective dose of the agent is able to cross the BBB when administered systemically. Administration of an inhibitor of a BBB key regulatory gene, e.g., an inhibitor of Mfsd2A, can increase the permeability of the BBB such that, e.g. a therapeutically effective dose of the CNS therapeutic agent is able to reach the CNS or the necessary dose of the CNS therapeutic agent can be lowered.

In some embodiments, a subject in need of increased permeability of the blood-brain barrier is in need of treatment for a condition selected from the group consisting of brain cancer; encephalitis; hydrocephalus; Parksinson's disease; neuropathic pain; and a condition treated by the administration of psychiatric drugs.

In some embodiments, an agonist of a BBB key regulatory gene, e.g., an agonist of Mfsd2A, can be administered to a subject in need of improved quality (e.g. decreased permeability) of tight junctions of the blood-brain barrier. In some embodiments, the subject in need of improved quality of tight junctions of the blood-brain barrier can be a subject who has been diagnosed with or determined to have abnormally high permeability of the blood-brain barrier, e.g. repeated infections of the CNS or in which abnormal levels of a systemically administered tracer molecule reach the CNS. In some embodiments, the subject in need of improved quality of tight junctions of the blood-brain barrier can be a subject in need of treatment (e.g. having, diagnosed as having, or at risk of developing) a condition selected from the group consisting of a neurodegenerative disease; multiple sclerosis; Parkinson's disease; Huntington's disease; Pick's disease; ALS; dementia; stoke; and Alzheimer's disease. In some embodiments, administration of the agonist of a BBB key regulatory gene, e.g., an agonist of Mfsd2A, can slow or halt the progression of a neurodegenerative disease. In some embodiments, administration of the agonist of a BBB key regulatory gene, e.g., the agonist of Mfsd2A, can slow or prevent the development of at least some symptoms of a neurodegenerative disease.

Tissue membranes other than the blood-brain barrier can be modulated in accordance with the methods described herein. For example, the permeability of the blood-retinal barrier or tissue membranes (e.g., tissue membranes comprising smooth muscle cells) can be modulated. In one aspect, described herein is a method of treatment, the method comprising administering an agonist of a gene or gene expression product selected from the group consisting of: Mfsd2A; Slco1C1; Slc38A5; LRP8; Slc3A2; Slc7A5; Slc7A1; Slc6A6; IGFBP7; Glut1; Slc40A1; and Slc30A1 to the subject in need of treatment for a retinal disease. In some embodiments, the agonist is an agonist of LRP8. In some embodiments, the agonist is a polypeptide or a nucleic acid encoding a polypeptide selected from the group consisting of: a Mfsd2A polypeptide; a Slco1C1 polypeptide; a Slc38A5 polypeptide; a LRP8 polypeptide; a Slc3A2 polypeptide; a Slc7A5 polypeptide; a Slc7A1 polypeptide; a Slc6A6 polypeptide; a IGFBP7 polypeptide; a Glut1 polypeptide; a Slc40A1 polypeptide; and a Slc30A1 polypeptide. In some embodiments, the subject administered an agonist is in need of improved quality of the retinal barrier. In some embodiments, the subject is in need of treatment for a condition selected from the group consisting of glaucoma; diabetic retinopathy; and age-related macular degeneration.

In one aspect, described herein is a method of modulating the permeability of a tissue membrane in a subject, the method comprising: administering an inhibitor of a gene or gene expression product selected from the group consisting of Mfsd2A; Slco1C1; Slc38A5; LRP8; Slc3A2; Slc7A5; Slc7A1; Slc6A6; IGFBP7; Glut1; Slc40A1; and Slc30A1 to the subject, whereby the permeability of the tissue membrane is increased; or administering an agonist of a gene or gene expression product selected from the group consisting of: Mfsd2A; Slco1C1; Slc38A5; LRP8; Slc3A2; Slc7A5; Slc7A1; Slc6A6; IGFBP7; Glut1; Slc40A1; and Slc30A1 to the subject, whereby the permeability of the tissue membrane is decreased. In one aspect, described herein is a method of treatment, the method comprising administering an inhibitor of a gene or gene expression product selected from the group consisting of Mfsd2A; Slco1C1; Slc38A5; LRP8; Slc3A2; Slc7A5; Slc7A1; Slc6A6; IGFBP7; Glut1; Slc40A1; and Slc30A1 to a subject in need of increased permeability of a tissue membrane; or administering an agonist of a gene or gene expression product selected from the group consisting of Mfsd2A; Slco1C1; Slc38A5; LRP8; Slc3A2; Slc7A5; Slc7A1; Slc6A6; IGFBP7; Glut1; Slc40A1; and Slc30A1 to the subject in need of decreased permeability of the tissue membrane. In some embodiments, the tissue membrane is selected from the group consisting of a kidney membrane; a placental membrane; or a testes membrane. In some embodiments, the inhibitor is selected from the group consisting of inhibitory antibodies and inhibitory nucleic acids. In some embodiments, the inhibitor is an inhibitor of Mfsd2A. In some embodiments, the inhibitor of Mfsd2A is selected from the group consisting of tunicamycin; tunicamycin analogs; inhibitory anti-Mfsd2A antibodies; and inhibitory nucleic acids. In some embodiments, the agonist is a polypeptide or a nucleic acid encoding a polypeptide selected from the group consisting of a Mfsd2A polypeptide; a Slco1C1 polypeptide; a Slc38A5 polypeptide; a LRP8 polypeptide; a Slc3A2 polypeptide; a Slc7A5 polypeptide; a Slc7A1 polypeptide; a Slc6A6 polypeptide; a IGFBP7 polypeptide; a Glut1 polypeptide; a Slc40A1 polypeptide; and a Slc30A1 polypeptide. In some embodiments, the subject in need of decreased permeability of the tissue membrane is in need of treatment for a condition selected from the group consisting of proteinuremia.

In one aspect, provided herein is an antibody reagent that binds to Mfsd2A. In some embodiments, the antibody reagent can bind specifically to Mfsd2A. In some embodiments, the antibody reagent can be an inhibitor of Mfsd2A.

In some embodiments, the antibody reagent can bind specifically to an epitope comprising the amino acid corresponding to residue 92 of SEQ ID NO: 3. In some embodiments, the antibody reagent can bind specifically to an epitope comprising the amino acid corresponding to residue 96 of SEQ ID NO: 3.

In some embodiments, the antibody reagent can bind specifically to an epitope comprising amino acids corresponding to residues 1-52 of SEQ ID NO: 3. In some embodiments, the antibody reagent can bind specifically to an epitope comprising at least 4 amino acids of the amino acids corresponding to residues 1-52 of SEQ ID NO: 3, e.g., 4 amino acids, 5 amino acids, 6 amino acids, 7 amino acids, 8 amino acids, or more amino acids comprised by that region of SEQ ID NO: 3. In some embodiments, the antibody reagent can bind specifically to an epitope comprising amino acids corresponding to residues 31-39 of SEQ ID NO: 3. In some embodiments, the antibody reagent can bind specifically to an epitope comprising at least 4 amino acids of the amino acids corresponding to residues 31-39 of SEQ ID NO: 3, e.g., 4 amino acids, 5 amino acids, 6 amino acids, 7 amino acids, 8 amino acids, or more amino acids comprised by that region of SEQ ID NO: 3. In some embodiments, the antibody reagent can bind specifically to an epitope comprising amino acids corresponding to residues 99-114 of SEQ ID NO: 3. In some embodiments, the antibody reagent can bind specifically to an epitope comprising at least 4 amino acids of the amino acids corresponding to residues 99-114 of SEQ ID NO: 3, e.g., 4 amino acids, 5 amino acids, 6 amino acids, 7 amino acids, 8 amino acids, or more amino acids comprised by that region of SEQ ID NO: 3. In some embodiments, the antibody reagent can bind specifically to an epitope comprising amino acids corresponding to residues 175-191 of SEQ ID NO: 3. In some embodiments, the antibody reagent can bind specifically to an epitope comprising at least 4 amino acids of the amino acids corresponding to residues 175-191 of SEQ ID NO: 3, e.g., 4 amino acids, 5 amino acids, 6 amino acids, 7 amino acids, 8 amino acids, or more amino acids comprised by that region of SEQ ID NO: 3. In some embodiments, the antibody reagent can bind specifically to an epitope comprising amino acids corresponding to residues 268-298 of SEQ ID NO: 3. In some embodiments, the antibody reagent can bind specifically to an epitope comprising at least 4 amino acids of the amino acids corresponding to residues 268-298 of SEQ ID NO: 3, e.g., 4 amino acids, 5 amino acids, 6 amino acids, 7 amino acids, 8 amino acids, or more amino acids comprised by that region of SEQ ID NO: 3. In some embodiments, the antibody reagent can bind specifically to an epitope comprising amino acids corresponding to residues 355-360 of SEQ ID NO: 3. In some embodiments, the antibody reagent can bind specifically to an epitope comprising at least 4 amino acids of the amino acids corresponding to residues 355-360 of SEQ ID NO: 3, e.g., 4 amino acids, 5 amino acids, 6 amino acids, 7 amino acids, 8 amino acids, or more amino acids comprised by that region of SEQ ID NO: 3. In some embodiments, the antibody reagent can bind specifically to an epitope comprising amino acids corresponding to residues 406-428 of SEQ ID NO: 3. In some embodiments, the antibody reagent can bind specifically to an epitope comprising at least 4 amino acids of the amino acids corresponding to residues 406-428 of SEQ ID NO: 3, e.g., 4 amino acids, 5 amino acids, 6 amino acids, 7 amino acids, 8 amino acids, or more amino acids comprised by that region of SEQ ID NO: 3. In some embodiments, the antibody reagent can bind specifically to an epitope comprising amino acids corresponding to residues 494-533 of SEQ ID NO: 3. In some embodiments, the antibody reagent can bind specifically to an epitope comprising at least 4 amino acids of the amino acids corresponding to residues 494-533 of SEQ ID NO: 3, e.g., 4 amino acids, 5 amino acids, 6 amino acids, 7 amino acids, 8 amino acids, or more amino acids comprised by that region of SEQ ID NO: 3. In some embodiments, the antibody reagent can bind specifically to an epitope comprising amino acids corresponding to residues 506-509 of SEQ ID NO: 3. In some embodiments, the antibody reagent can bind specifically to an epitope comprising at least 4 amino acids of the amino acids corresponding to residues 506-509 of SEQ ID NO: 3, e.g., 4 amino acids, 5 amino acids, 6 amino acids, 7 amino acids, 8 amino acids, or more amino acids comprised by that region of SEQ ID NO: 3.

In some embodiments, the antibody reagent can bind specifically to an epitope comprising amino acids corresponding to residues 74-77 of SEQ ID NO: 3. In some embodiments, the antibody reagent can bind specifically to an epitope comprising amino acids corresponding to residues 136-150 of SEQ ID NO: 3. In some embodiments, the antibody reagent can bind specifically to an epitope comprising at least 4 amino acids of the amino acids corresponding to residues 136-150 of SEQ ID NO: 3, e.g., 4 amino acids, 5 amino acids, 6 amino acids, 7 amino acids, 8 amino acids, or more amino acids comprised by that region of SEQ ID NO: 3. In some embodiments, the antibody reagent can bind specifically to an eptiope comprising amino acids corresponding to residues 214-246 of SEQ ID NO: 3. In some embodiments, the antibody reagent can bind specifically to an epitope comprising at least 4 amino acids of the amino acids corresponding to residues 214-246 of SEQ ID NO: 3, e.g., 4 amino acids, 5 amino acids, 6 amino acids, 7 amino acids, 8 amino acids, or more amino acids comprised by that region of SEQ ID NO: 3. In some embodiments, the antibody reagent can bind specifically to an epitope comprising amino acids corresponding to residues 319-331 of SEQ ID NO: 3. In some embodiments, the antibody reagent can bind specifically to an epitope comprising at least 4 amino acids of the amino acids corresponding to residues 319-331 of SEQ ID NO: 3, e.g., 4 amino acids, 5 amino acids, 6 amino acids, 7 amino acids, 8 amino acids, or more amino acids comprised by that region of SEQ ID NO: 3. In some embodiments, the antibody reagent can bind specifically to an epitope comprising amino acids corresponding to residues 382-384 of SEQ ID NO: 3. In some embodiments, the antibody reagent can bind specifically to an epitope comprising amino acids corresponding to residues 448-472 of SEQ ID NO: 3. In some embodiments, the antibody reagent can bind specifically to an epitope comprising at least 4 amino acids of the amino acids corresponding to residues 448-472 of SEQ ID NO: 3, e.g., 4 amino acids, 5 amino acids, 6 amino acids, 7 amino acids, 8 amino acids, or more amino acids comprised by that region of SEQ ID NO: 3.

Agents which bind to the BBB key regulatory genes described herein can, after such binding, be endocytosed into the cell expressing the key regulatory gene. When the binding agent is present in a composition and/or conjugated to one or more additional agents, the endocytosis can permit of the additional agent(s) into the cell, i.e. compositions comprising an agent that binds a BBB key regulatory gene can be transported across the BBB. In one aspect, described herein is a pharmaceutical composition comprising a) an antibody reagent that binds to a polypeptide selected from the group consisting of: Mfsd2A; Slco1C1; Slc38A5; LRP8; Slc3A2; Slc7A5; Slc7A1; Slc6A6; IGFBP7; Glut1; Slc40A1; and Slc30A1; b) a central nervous system therapeutic agent; and a pharmaceutically-acceptable carrier. In one aspect, described herein is a method of treatment, the method comprising administering to a subject in need of a central nervous system therapeutic agent a composition comprising a) an antibody reagent that binds to a polypeptide selected from the group consisting of: Mfsd2A; Slco1C1; Slc38A5; LRP8; Slc3A2; Slc7A5; Slc7A1; Slc6A6; IGFBP7; Glut1; Slc40A1; and Slc30A1; and b) a central nervous system therapeutic agent. Central nervous system therapeutic agents are described elsewhere herein.

In some embodiments, the central nervous system therapeutic reagent is less than about 70 kDa in size. In some embodiments, the central nervous system therapeutic reagent is less than 70 kDa in size. In some embodiments, the central nervous system therapeutic reagent and the antibody reagent are, in combination, less than about 70 kDa in size. In some embodiments, the central nervous system therapeutic reagent and the antibody reagent are, in combination, less than 70 kDa in size.

In some embodiments, the antibody reagent that binds a BBB key regulatory gene can be an inhibitor of the BBB key regulatory gene. In some embodiments, the antibody reagent that binds a BBB key regulatory gene can be an agonist of the BBB key regulatory gene. In some embodiments, the antibody reagent that binds a BBB key regulatory gene has no detectable effect on the level and/or activity of the BBB key regulatory gene. In some embodiments, the antibody reagent that binds a BBB key regulatory gene has no statistically significant effect on the level and/or activity of the BBB key regulatory gene. Antibody reagents are discussed elsewhere herein.

In some embodiments, the composition comprises a bi-specific antibody, e.g. an antibody that can specifically bind to both a BBB key regulatory gene and a therapeutic target. The therapeutic target can vary depending upon the disease to be treated. Targets for various diseases of the CNS are known in the art, see, e.g. Corbo and Alsono Adel. Prog Mol Biol Transl Sci 2011 98:47-83 and “Emerging Drugs and Targets for Alzheimer's Diesease” Martinez (ed), 2010, RSC Press for discussion of Alzheimer's targets; Hickey and Stacy. Drug Des Devel Thera 2011 5:241-254; Coune et al. Cold Sprin Harb Perspect Med 2012 2:a009431; and Douglas. Expert Review of Neurotherapeutics 2013 13:695-705 for discussion of Parkinson's disease targets. In some embodiments, the subject is in need of treatment for a condition selected from the group consisting of brain cancer; encephalitis; hydrocephalus; Parksinson's disease; neuropathic pain; a condition treated by the administration of psychiatric drugs; a neurodegenerative disease; multiple sclerosis; Huntington's disease; Pick's disease; ALS; dementia; stroke; and Alzheimer's disease. In some embodiments, the subject is in need of treatment for Alzheimer's, and the therapeutic target is beta-secretase 1.

In some embodiments, the composition can comprise a peptibody, F′ab fragment, recombinant polypeptides, and/or a ligand of one or both of the BB key regulatory gene and the therapeutic target.

In some embodiments, the antibody reagent which binds to the BBB key regulatory gene polypeptide and the therapeutic agent can be directly conjugated and/or bound to each other, e.g. an antibody-drug conjugate. In some embodiments, binding can be non-covalent, e.g., by hydrogen, electrostatic, or van der waals interactions, however, binding may also be covalent. By “conjugated” is meant the covalent linkage of at least two molecules. In some embodiments, the composition can be an antibody-drug conjugate. In some embodiments, the antibody reagent can be bound to and/or conjugated to multiple therapeutic molecules. In some embodiments, the ratio of a given therapeutic molecule to the antibody reagent molecule can be from about 1:1 to about 1,000:1, e.g. a single antibody reagent molecule can be linked to, conjugated to, etc. from about 1 to about 1,000 individual therapeutic molecules.

In some embodiments, the antibody reagent which binds to the BBB key regulatory gene polypeptide and the therapeutic agent can be present in a scaffold material. Scaffold materials suitable for use in therapeutic compositions are known in the art and can include, but are not limited to, a nanoparticle; a matrix; a hydrogel; and a biomaterial, biocompatible, and/or biodegradable scaffold material. As used herein, the term “nanoparticle” refers to particles that are on the order of about 10⁻⁹ or one billionth of a meter. The term “nanoparticle” includes nanospheres; nanorods; nanoshells; and nanoprisms; and these nanoparticles may be part of a nanonetwork.

The term “nanoparticles” also encompasses liposomes and lipid particles having the size of a nanoparticle. As used herein, the term “matrix” refers to a 3-dimensional structure comprising the components of a composition described herein (e.g. a binding reagent, kinase inhibitor, and/or EGFR inhibitor). Non-limiting examples of matrix structures include foams; hydrogels; electrospun fibers; gels; fiber mats; sponges; 3-dimensional scaffolds; non-woven mats; woven materials; knit materials; fiber bundles; and fibers and other material formats (See, e.g. Rockwood et al. Nature Protocols 2011 6:1612-1631 and US Patent Publications 2011/0167602; 2011/0009960; 2012/0296352; and U.S. Pat. No. 8,172,901; each of which is incorporated by reference herein in its entirety). The structure of the matrix can be selected by one of skill in the art depending upon the intended application of the composition, e.g. electrospun matrices can have greater surface area than foams.

In some embodiments, the scaffold is a hydrogel. As used herein, the term “hydrogel” refers to a three-dimensional polymeric structure that is insoluble in water but which is capable of absorbing and retaining large quantities of water to form a stable, often soft and pliable, structure. In some embodiments, water can penetrate in between the polymer chains of the polymer network, subsequently causing swelling and the formation of a hydrogel. In general, hydrogels are superabsorbent. Hydrogels have many desirable properties for biomedical applications. For example, they can be made nontoxic and compatible with tissue, and they are highly permeable to water, ions, and small molecules. Hydrogels are super-absorbent (they can contain over 99% water) and can be comprised of natural (e.g., silk) or synthetic polymers, e.g., PEG.

As used herein, “biomaterial” refers to a material that is biocompatible and biodegradable. As used herein, the term “biocompatible” refers to substances that are not toxic to cells. In some embodiments, a substance is considered to be “biocompatible” if its addition to cells in vitro results in less than or equal to approximately 20% cell death. In some embodiments, a substance is considered to be “biocompatible” if its addition to cells in vivo does not induce inflammation and/or other adverse effects in vivo. As used herein, the term “biodegradable” refers to substances that are degraded under physiological conditions. In some embodiments, a biodegradable substance is a substance that is broken down by cellular machinery. In some embodiments, a biodegradable substance is a substance that is broken down by chemical processes.

In some embodiments, the methods described herein relate to treating a subject having or diagnosed as having a disease affecting the CNS, e.g. a neurodegenerative disease or a condition treated by delivering therapeutic agents to the CNS. Subjects having a disease affecting the CNS can be identified by a physician using current methods of diagnosing such conditions. Symptoms and/or complications of such conditions which characterize these conditions and aid in diagnosis are well known in the art and include but are not limited to, lost of neural function (e.g. lack of coordination, lack of sensation, altered behaviors, inflammation of the CNS, headaches, etc). Tests that may aid in a diagnosis of such conditions can include, but are not limited to, CT scan, MRI scan, spinal tap, brain biopsy, electroencephalogram (EEG), lumbar puncture, and/or blood tests. For some conditions, a family history of the condition, or exposure to risk factors for the condition can also aid in determining if a subject is likely to have the condition or in making a diagnosis.

The compositions and methods described herein can be administered to a subject having or diagnosed as having a disease affecting the CNS. In some embodiments, the methods described herein comprise administering an effective amount of compositions described herein, to a subject in order to alleviate a symptom of a disease affecting the CNS. As used herein, “alleviating a symptom” is ameliorating any condition or symptom associated with the disease affecting the CNS. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, intravenous, intramuscular, subcutaneous, transdermal, airway (aerosol), pulmonary, cutaneous, injection, or intratumoral administration. Administration can be local or systemic.

The term “effective amount” as used herein refers to the amount of a composition needed to alleviate at least one or more symptom of the disease or disorder, and relates to a sufficient amount of pharmacological composition to provide the desired effect. The term “therapeutically effective amount” therefore refers to an amount of a composition that is sufficient to provide a particular effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the active agent which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for BBB permeability, among others. The dosage can be determined by a physician and adjusted, as necessary, to suit observed effects of the treatment.

In some embodiments, the technology described herein relates to a pharmaceutical composition comprising an agonist or inhibitor of a BBB key regulatory gene, e.g., Mfsd2A, as described herein, and optionally a pharmaceutically acceptable carrier.

In one aspect, described herein is a pharmaceutical composition comprising an inhibitor of a BBB key regulatory gene, e.g., an inhibitor of Mfsd2A, and a pharmaceutically-acceptable carrier. In some embodiments, the inhibitor of a BBB key regulatory gene, e.g., an inhibitor of Mfsd2A is selected from the group consisting of tunicamycin; tunicamycin analogs; inhibitory anti-BBB key regulatory gene antibodies; inhibitory and nucleic acids. In some embodiments, the composition can further comprise a central nervous system therapeutic agent.

In one aspect, described herein is a pharmaceutical composition comprising an agonist of a BBB key regulatory gene, e.g., an agonist of Mfsd2A, and a pharmaceutically-acceptable carrier. In some embodiments, the agonist of a BBB key regulatory gene, e.g., the agonist of Mfsd2A, is selected from the group consisting of a BBB key regulatory gene polypeptide and an nucleic acid encoding a BBB key regulatory gene polypeptide, e.g., a Mfsd2A polypeptide; and a nucleic acid encoding a Mfsd2A polypeptide.

Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agent.

In some embodiments, the pharmaceutical composition as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Suitable vehicles that can be used to provide parenteral dosage forms of a composition as disclosed within are well known to those skilled in the art. Examples include, without limitation: sterile water; water for injection USP; saline solution; glucose solution; aqueous vehicles such as but not limited to, sodium chloride injection, Ringer's injection, dextrose Injection, dextrose and sodium chloride injection, and lactated Ringer's injection; water-miscible vehicles such as, but not limited to, ethyl alcohol, polyethylene glycol, and propylene glycol; and non-aqueous vehicles such as, but not limited to, corn oil, cottonseed oil, peanut oil, sesame oil, ethyl oleate, isopropyl myristate, and benzyl benzoate. Compounds that alter or modify the solubility of a pharmaceutically acceptable salt can also be incorporated into the parenteral dosage forms of the disclosure, including conventional and controlled-release parenteral dosage forms.

Pharmaceutical compositions can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, aerosol sprays, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia Pa. (2005).

Conventional dosage forms generally provide rapid or immediate drug release from the formulation. Depending on the pharmacology and pharmacokinetics of the drug, use of conventional dosage forms can lead to wide fluctuations in the concentrations of the drug in a patient's blood and other tissues. These fluctuations can impact a number of parameters, such as dose frequency, onset of action, duration of efficacy, maintenance of therapeutic blood levels, toxicity, side effects, and the like. Advantageously, controlled-release formulations can be used to control a drug's onset of action, duration of action, plasma levels within the therapeutic window, and peak blood levels. In particular, controlled- or extended-release dosage forms or formulations can be used to ensure that the maximum effectiveness of a drug is achieved while minimizing potential adverse effects and safety concerns, which can occur both from under-dosing a drug (i.e., going below the minimum therapeutic levels) as well as exceeding the toxicity level for the drug. In some embodiments, the composition can be administered in a sustained release formulation.

Controlled-release pharmaceutical products have a common goal of improving drug therapy over that achieved by their non-controlled release counterparts. Ideally, the use of an optimally designed controlled-release preparation in medical treatment is characterized by a minimum of drug substance being employed to cure or control the condition in a minimum amount of time. Advantages of controlled-release formulations include: 1) extended activity of the drug; 2) reduced dosage frequency; 3) increased patient compliance; 4) usage of less total drug; 5) reduction in local or systemic side effects; 6) minimization of drug accumulation; 7) reduction in blood level fluctuations; 8) improvement in efficacy of treatment; 9) reduction of potentiation or loss of drug activity; and 10) improvement in speed of control of diseases or conditions. Kim, Cherng-ju, Controlled Release Dosage Form Design, 2 (Technomic Publishing, Lancaster, Pa.: 2000).

Most controlled-release formulations are designed to initially release an amount of drug (active ingredient) that promptly produces the desired therapeutic effect, and gradually and continually release other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, ionic strength, osmotic pressure, temperature, enzymes, water, and other physiological conditions or compounds.

A variety of known controlled- or extended-release dosage forms, formulations, and devices can be adapted for use with the salts and compositions of the disclosure. Examples include, but are not limited to, those described in U.S. Pat. Nos. 3,845,770; 3,916,899; 3,536,809; 3,598,123; 4,008,719; 5,674,533; 5,059,595; 5,591,767; 5,120,548; 5,073,543; 5,639,476; 5,354,556; 5,733,566; and 6,365,185 B1; each of which is incorporated herein by reference. These dosage forms can be used to provide slow or controlled-release of one or more active ingredients using, for example, hydroxypropylmethyl cellulose, other polymer matrices, gels, permeable membranes, osmotic systems (such as OROS® (Alza Corporation, Mountain View, Calif. USA)), or a combination thereof to provide the desired release profile in varying proportions.

The methods described herein can further comprise administering a second agent and/or treatment to the subject, e.g. as part of a combinatorial therapy. Non-limiting examples of a second agent and/or treatment can include CNS therapeutic agents as described herein, agents for the treatment of neurodegenerative diseases, and/or agents to treat symptoms or complications of any of the conditions described herein. Further, the methods of treatment can further include the use of surgical treatments.

In certain embodiments, an effective dose of a composition as described herein can be administered to a patient once. In certain embodiments, an effective dose of a composition can be administered to a patient repeatedly. For systemic administration, subjects can be administered a therapeutic amount of a composition, such as, e.g. 0.1 mg/kg, 0.5 mg/kg, 1.0 mg/kg, 2.0 mg/kg, 2.5 mg/kg, 5 mg/kg, 10 mg/kg, 15 mg/kg, 20 mg/kg, 25 mg/kg, 30 mg/kg, 40 mg/kg, 50 mg/kg, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the active agent. The desired dose or amount of effect can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition can be administered over a period of time, such as over a 5 minute, 10 minute, 15 minute, 20 minute, or 25 minute period.

The dosage ranges for the administration of a composition, according to the methods described herein depend upon, for example, the form of the composition, its potency, and the extent to which symptoms, markers, or indicators of a condition described herein are desired to be reduced, for example the percentage modulation desired for permeability of the BBB. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a composition in, e.g. the treatment of a condition described herein, or to induce a response as described herein (e.g. modulation of BBB permeability) can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. BBB permeability to a detectable agent as described herein. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response, (e.g. BBB permeability, or symptoms of a disease affecting the CNS). It is well within the ability of one skilled in the art to monitor efficacy of administration and/or treatment by measuring any one of such parameters, or any combination of parameters. Efficacy can be assessed in animal models of a condition described herein, for example treatment of a disease affecting the CNS of a mouse, or the mouse embryo model of BBB permeability described herein. When using an experimental animal model, efficacy of treatment is evidenced when a statistically significant change in a marker is observed.

In vitro and animal model assays are provided herein which allow the assessment of a given dose of a composition. The efficacy of a given dosage combination can also be assessed in an animal model, e.g. a mouse embryo in the assays described herein.

In one aspect, described herein is a method for determining the permeability of the blood-brain barrier during development, the method comprising injecting the liver of an embryo with a detectable agent while the embryo is connected to the maternal circulation via the umbilical cord allowing the dye to circulate in the bloodstream and detecting a signal from the detectable agent in blood vessels within the brain and within brain tissue separated from the bloodstream by the blood-brain barrier.

In some embodiments, the embryo can be a murine embryo. In some embodiments, the murine embryo can be less than 19 days of age, eg. 19 days or less, 18 days or less, 17 days or less, 16 days or less, 15 days or less, 14 days or less, or 13 days or less of age. In some embodiments, the murine embryo can be at least 10 days of age, e.g. at least 10 days, at least 11 days, at least 12 days, at least 13 days, at least 14 days, or at least 15 days of age. In some embodiments, the murine embryo can be from about 10 days to about 19 days of age.

A detectable agent can be any agent that produces or can be caused to produce a detectable signal, e.g. an agent with a detectable label. In some embodiments, the detectable agent can be a fixable dye. In some embodiments, the detectable agent can be a dye which is fixable by immersion fixation. Non-limiting examples of suitable dyes can include Evans blue, Hochset, biotin, HRP, and fluoro-Ruby-Dextran. In some embodiments, the agent can be fluoro-Ruby-Dextran.

The volume of the injection comprising the detectable agent should be low enough that the pressure within the circulatory system does not cause capillaries to rupture. In some embodiments, the total volume of the injection is less than or equal to 1 uL for a murine embryo of about 13.5 days age, less than or equal to 2 uL for a murine embryo of about 14.5 days of age, and less than or equal to 5 uL for a murine embryo of about 15 days of age or older. One of skill in the art can readily convert the foregoing volumes for use with embryos of different ages and/or species by comparing the known sizes and rates of development of a murine embryo with the embryo of interest.

In some embodiments, the detectable agent is allowed to circulate for from about 10 seconds to about 3 hours. In some embodiments, the detectable agent is allowed to circulate for from about 30 seconds to about 30 minutes. In some embodiments, the detectable agent is allowed to circulate for from about 1 minute to about 20 minutes. In some embodiments, the detectable agent is allowed to circulate for from about 1 minute to about 10 minutes. In some embodiments, the detectable agent is allowed to circulate for from about 5 minutes to about 30 minutes. In some embodiments, the detectable agent is allowed to circulate for from about 5 minutes to about 30 minutes in an adult animal. In some embodiments, the detectable agent is allowed to circulate for from about 3 minutes to about 5 minutes. In some embodiments, the detectable agent is allowed to circulate in an embryo for from about 3 minutes to about 5 minutes. In some embodiments, the detectable agent is allowed to circulate for about 3 minutes. When the detectable agent has been allowed to circulate for the desired time, the embryo can be fixed and/or a detectable signal from the agent can be measured.

Methods of detecting various types of detectable labels are well known in the art, e.g. fluorescent microscopy to detect a fluorescent label. The amount of signal present, e.g. in the circulatory system, the CNS, and/or elsewhere in the embryo can be measured by, e.g. scoring an image or via computer programs that can quantitate the amount and/or intensity of a signal in a given area of an image. Such methods are known in the art.

In one aspect, described herein is a method for identifying a modulator of the permeability of the blood-brain barrier during development, the method comprising administering a candidate modulator agent to an embryo, injecting the liver of an embryo with a detectable agent while the embryo is connected to the maternal circulation via the umbilical cord, allowing the dye to circulate in the bloodstream, detecting a signal from the detectable agent in blood vessels within the brain and within brain tissue separated from the bloodstream by the blood-brain barrier, wherein the candidate modulator is determined to increase permeability of the blood-brain barrier if the ratio of signal detected in brain tissue:signal detected in the blood vessels within the brain is lower than a reference level; and wherein the candidate modulator is determined to decrease permeability of the blood-brain barrier if the ratio of signal detected in brain tissue:signal detected in the blood vessels within the brain is higher than a reference level.

Detectable agents and methods of detecting the signal from a detectable agent are described elsewhere herein.

As used herein, a “candidate agent” refers to any entity which is normally not present or not present at the levels being administered to a cell, tissue or subject. A candidate agent can be selected from a group comprising: chemicals; small organic or inorganic molecules; nucleic acid sequences; nucleic acid analogues; proteins; peptides; aptamers; peptidomimetic, peptide derivative, peptide analogs, antibodies; intrabodies; biological macromolecules, extracts made from biological materials such as bacteria, plants, fungi, or animal cells or tissues; naturally occurring or synthetic compositions or functional fragments thereof. In some embodiments, the candidate agent is any chemical, entity or moiety, including without limitation synthetic and naturally-occurring non-proteinaceous entities. In certain embodiments the candidate agent is a small molecule having a chemical moiety. For example, chemical moieties include unsubstituted or substituted alkyl, aromatic, or heterocyclyl moieties including macrolides, leptomycins and related natural products or analogues thereof. Candidate agents can be known to have a desired activity and/or property, or can be selected from a library of diverse compounds.

Generally, compounds can be tested at any concentration that can modulate the permeability of the blood-brain barrier relative to a control over an appropriate time period. In some embodiments, compounds are tested at concentration in the range of about 0.1 nM to about 1000 mM. In one embodiment, the compound is tested in the range of about 0.1 μM to about 20 μM, about 0.1 μM to about 10 μM, or about 0.1 μM to about 5 μM.

Depending upon the particular embodiment being practiced, the candidate or test agents can be provided free in solution. Additionally, for the methods described herein, test compounds can be screened individually, or in groups. Group screening is particularly useful where hit rates for effective test compounds are expected to be low such that one would not expect more than one positive result for a given group.

Methods for developing small molecule, polymeric and genome based libraries are described, for example, in Ding, et al. J Am. Chem. Soc. 124: 1594-1596 (2002) and Lynn, et al., J. Am. Chem. Soc. 123: 8155-8156 (2001). Commercially available compound libraries can be obtained from, e.g., ArQule (Woburn, Mass.), Invitrogen (Carlsbad, Calif.), Ryan Scientific (Mt. Pleasant, S.C.), and Enzo Life Sciences (Farmingdale, N.Y.). These libraries can be screened for the ability of members to modulate the BBB using e.g. methods described herein.

In some embodiments, the candidate agents can be naturally occurring proteins or their fragments. Such candidate agents can be obtained from a natural source, e.g., a cell or tissue lysate. Libraries of polypeptide agents can also be prepared, e.g., from a cDNA library commercially available or generated with routine methods. The candidate agents can also be peptides, e.g., peptides of from about 5 to about 30 amino acids, with from about 5 to about 20 amino acids being preferred and from about 7 to about 15 being particularly preferred. The peptides can be digests of naturally occurring proteins, random peptides, or “biased” random peptides. In some methods, the candidate agents are polypeptides or proteins. Peptide libraries, e.g. combinatorial libraries of peptides or other compounds can be fully randomized, with no sequence preferences or constants at any position. Alternatively, the library can be biased, i.e., some positions within the sequence are either held constant, or are selected from a limited number of possibilities. For example, in some cases, the nucleotides or amino acid residues are randomized within a defined class, for example, of hydrophobic amino acids, hydrophilic residues, sterically biased (either small or large) residues, towards the creation of cysteines, for cross-linking, prolines for SH-3 domains, serines, threonines, tyrosines or histidines for phosphorylation sites, or to purines.

The candidate agents can also be nucleic acids. Nucleic acid candidate agents can be naturally occurring nucleic acids, random nucleic acids, or “biased” random nucleic acids. For example, digests of prokaryotic or eukaryotic genomes can be similarly used as described above for proteins.

The candidate agent can function directly in the form in which it is administered. Alternatively, the candidate agent can be modified or utilized intracellularly to produce a form that modulates the desired activity, e.g. introduction of a nucleic acid sequence into a cell and its transcription resulting in the production of an inhibitor or activator of gene expression or protein activity within the cell.

In some embodiments, the candidate agent that is screened and identified to modulate the permeability of the BBB according to the methods described herein by at least 5%, preferably at least 10%, 20%, 30%, 40%, 50%, 50%, 70%, 80%, 90% relative to an untreated control. A level which is higher or lower than a reference level (e.g. the level in the absence of the candidate agent) can be a level which is statistically significantly different than the reference level. In some embodiments, a level that is lower than a reference level can be 90% or less of the reference level, e.g. 90% or less, 80% or less, 70% or less, 60% or less, 50% or less, 25% or less, or 10% or less of the reference level. In some embodiments, a level that is higher than a reference level can be 1.5× or more of the reference level, e.g. 1.5× or more, 2× or more, 3× or more, 5× or more, or 10× or more of the reference level.

The reference level can be the level in the absence of the candidate agent, e.g. the level in a parallel, untreated embryo, the level in the embryo prior to contact with the candidate agent, and/or a level in a population of embryos not contacted with the agent, e.g. a pre-determined level.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of CNS diseases. A subject can be male or female.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

As used herein, the terms “protein” and “polypeptide” are used interchangeably herein to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

As used herein an “antibody” refers to IgG, IgM, IgA, IgD or IgE molecules or antigen-specific antibody fragments thereof (including, but not limited to, a Fab, F(ab′)₂, Fv, disulphide linked Fv, scFv, single domain antibody, closed conformation multispecific antibody, disulphide-linked scfv, diabody), whether derived from any species that naturally produces an antibody, or created by recombinant DNA technology; whether isolated from serum, B-cells, hybridomas, transfectomas, yeast or bacteria.

As described herein, an “antigen” is a molecule that is bound by a binding site on an antibody agent. Typically, antigens are bound by antibody ligands and are capable of raising an antibody response in vivo. An antigen can be a polypeptide, protein, nucleic acid or other molecule or portion thereof. The term “antigenic determinant” refers to an epitope on the antigen recognized by an antigen-binding molecule, and more particularly, by the antigen-binding site of said molecule.

As used herein, the term “antibody reagent” refers to a polypeptide that includes at least one immunoglobulin variable domain or immunoglobulin variable domain sequence and which specifically binds a given antigen. An antibody reagent can comprise an antibody or a polypeptide comprising an antigen-binding domain of an antibody. In some embodiments, an antibody reagent can comprise a monoclonal antibody or a polypeptide comprising an antigen-binding domain of a monoclonal antibody. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody reagent” encompasses antigen-binding fragments of antibodies (e.g., single chain antibodies, Fab and sFab fragments, F(ab′)2, Fd fragments, Fv fragments, scFv, and domain antibodies (dAb) fragments (see, e.g. de Wildt et al., Eur J. Immunol 1996; 26(3):629-39; which is incorporated by reference herein in its entirety)) as well as complete antibodies. An antibody can have the structural features of IgA, IgG, IgE, IgD, IgM (as well as subtypes and combinations thereof). Antibodies can be from any source, including mouse, rabbit, pig, rat, and primate (human and non-human primate) and primatized antibodies. Antibodies also include midibodies, humanized antibodies, chimeric antibodies, and the like.

The VH and VL regions can be further subdivided into regions of hypervariability, termed “complementarity determining regions” (“CDR”), interspersed with regions that are more conserved, termed “framework regions” (“FR”). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917; which are incorporated by reference herein in their entireties). Each VH and VL is typically composed of three CDRs and four FRs, arranged from amino-terminus to carboxy-terminus in the following order: FR1, CDR1, FR2, CDR2, FR3, CDR3, FR4.

The terms “antigen-binding fragment” or “antigen-binding domain”, which are used interchangeably herein are used to refer to one or more fragments of a full length antibody that retain the ability to specifically bind to a target of interest. Examples of binding fragments encompassed within the term “antigen-binding fragment” of a full length antibody include (i) a Fab fragment, a monovalent fragment consisting of the VL, VH, CL and CH1 domains; (ii) a F(ab′)2 fragment, a bivalent fragment including two Fab fragments linked by a disulfide bridge at the hinge region; (iii) an Fd fragment consisting of the VH and CH1 domains; (iv) an Fv fragment consisting of the VL and VH domains of a single arm of an antibody, (v) a dAb fragment (Ward et al., (1989) Nature 341:544-546; which is incorporated by reference herein in its entirety), which consists of a VH or VL domain; and (vi) an isolated complementarity determining region (CDR) that retains specific antigen-binding functionality. As used herein, the term “specific binding” refers to a chemical interaction between two molecules, compounds, cells and/or particles wherein the first entity binds to the second, target entity with greater specificity and affinity than it binds to a third entity which is a non-target. In some embodiments, specific binding can refer to an affinity of the first entity for the second target entity which is at least 10 times, at least 50 times, at least 100 times, at least 500 times, at least 1000 times or greater than the affinity for the third nontarget entity.

Additionally, and as described herein, a recombinant humanized antibody can be further optimized to decrease potential immunogenicity, while maintaining functional activity, for therapy in humans. In this regard, functional activity means a polypeptide capable of displaying one or more known functional activities associated with a recombinant antibody or antibody reagent thereof as described herein. Such functional activities include, e.g. the ability to bind to Mfsd2A.

The term “chimeric antibody” refers to antibodies which contain sequences for the variable region of the heavy and light chains from one species and constant region sequences from another species, such as antibodies having murine heavy and light chain variable regions linked to human constant regions. Humanized antibodies have variable region framework residues substantially from a human antibody (termed an acceptor antibody) and complementarity determining regions substantially from a non-human antibody, e.g. a mouse-antibody, (referred to as the donor immunoglobulin). See, Queen et al., Proc Natl Acad Sci USA 86:10029-10033 (1989) and WO 90/07861, U.S. Pat. No. 5,693,762, U.S. Pat. No. 5,693,761, U.S. Pat. No. 5,585,089, U.S. Pat. No. 5,530,101 and Winter, U.S. Pat. No. 5,225,539, which are herein incorporated by reference in their entirety. The constant region(s), if present, are also substantially or entirely from a human immunoglobulin. The human variable domains are usually chosen from human antibodies whose framework sequences exhibit a high degree of sequence identity with the (murine) variable region domains from which the CDRs were derived. The heavy and light chain variable region framework residues can be substantially similar to a region of the same or different human antibody sequences. The human antibody sequences can be the sequences of naturally occurring human antibodies or can be consensus sequences of several human antibodies. See Carter et al., WO 92/22653, which is herein incorporated by reference in its entirety.

In some embodiments, the antibody reagents (e.g. antibodies) described herein are not naturally-occurring biomolecules. For example, a murine antibody raised against an antigen of human origin would not occur in nature absent human intervention and manipulation, e.g. manufacturing steps carried out by a human. Chimeric antibodies are also not naturally-occurring biomolecules, e.g., in that they comprise sequences obtained from multiple species and assembled into a recombinant molecule. In some embodiments, the human antibody reagents described herein are not naturally-occurring biomolecules, e.g., fully human antibodies directed against a human antigen would be subject to negative selection in nature and are not naturally found in the human body.

Traditionally, monoclonal antibodies have been produced as native molecules in murine hybridoma lines. In addition to that technology, the methods and compositions described herein provide for recombinant DNA expression of monoclonal antibodies. This allows the production of humanized antibodies as well as a spectrum of antibody derivatives and fusion proteins in a host species of choice. The production of antibodies in bacteria, yeast, transgenic animals and chicken eggs are also alternatives for hybridoma-based production systems. The main advantages of transgenic animals are potential high yields from renewable sources.

Nucleic acid molecules encoding amino acid sequence variants of antibodies are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the antibody. A nucleic acid sequence encoding at least one antibody, portion or polypeptide as described herein can be recombined with vector DNA in accordance with conventional techniques, including blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulations are disclosed, e.g., by Maniatis et al., Molecular Cloning, Lab. Manual (Cold Spring Harbor Lab. Press, NY, 1982 and 1989), and Ausubel, 1987, 1993, and can be used to construct nucleic acid sequences which encode a monoclonal antibody molecule or antigen binding region thereof.

A nucleic acid molecule, such as DNA, is said to be “capable of expressing” a polypeptide if it contains nucleotide sequences which contain transcriptional and translational regulatory information and such sequences are “operably linked” to nucleotide sequences which encode the polypeptide. An operable linkage is a linkage in which the regulatory DNA sequences and the DNA sequence sought to be expressed are connected in such a way as to permit gene expression as peptides or antibody portions in recoverable amounts. The precise nature of the regulatory regions needed for gene expression may vary from organism to organism, as is well known in the analogous art. See, e.g., Sambrook et al., 1989; Ausubel et al., 1987-1993.

Accordingly, the expression of an antibody or antigen-binding portion thereof as described herein can occur in either prokaryotic or eukaryotic cells. Suitable hosts include bacterial or eukaryotic hosts, including yeast, insects, fungi, bird and mammalian cells either in vivo, or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of an antibody or portion thereof as described herein with a specified amino terminus sequence. Moreover, problems associated with retention of initiation codon-derived methionine residues in direct yeast (or bacterial) expression maybe avoided. Sabin et al., 7 Bio/Technol. 705 (1989); Miller et al., 7 Bio/Technol. 698 (1989). Any of a series of yeast gene expression systems incorporating promoter and termination elements from the actively expressed genes coding for glycolytic enzymes produced in large quantities when yeast are grown in mediums rich in glucose can be utilized to obtain recombinant antibodies or antigen-binding portions thereof as described herein. Known glycolytic genes can also provide very efficient transcriptional control signals. For example, the promoter and terminator signals of the phosphoglycerate kinase gene can be utilized.

Production of antibodies or antigen-binding portions thereof as described herein in insects can be achieved. For example, by infecting the insect host with a baculovirus engineered to express a transmembrane polypeptide by methods known to those of skill. See Ausubel et al., 1987, 1993.

In some embodiments, the introduced nucleotide sequence is incorporated into a plasmid or viral vector capable of autonomous replication in the recipient host. Any of a wide variety of vectors can be employed for this purpose and are known and available to those or ordinary skill in the art. See, e.g., Ausubel et al., 1987, 1993. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Example prokaryotic vectors known in the art include plasmids such as those capable of replication in E. coli, for example. Other gene expression elements useful for the expression of cDNA encoding antibodies or antigen-binding portions thereof include, but are not limited to (a) viral transcription promoters and their enhancer elements, such as the SV40 early promoter. (Okayama et al., 3 Mol. Cell. Biol. 280 (1983)), Rous sarcoma virus LTR (Gorman et al., 79 PNAS 6777 (1982)), and Moloney murine leukemia virus LTR (Grosschedl et al., 41 Cell 885 (1985)); (b) splice regions and polyadenylation sites such as those derived from the SV40 late region (Okayarea et al., 1983), and (c) polyadenylation sites such as in SV40 (Okayama et al., 1983) Immunoglobulin cDNA genes can be expressed as described by Liu et al., infra, and Weidle et al., 51 Gene 21 (1987), using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements.

For immunoglobulin genes comprised of part cDNA, part genomic DNA (Whittle et al., 1 Protein Engin. 499 (1987)), the transcriptional promoter can be human cytomegalovirus, the promoter enhancers can be cytomegalovirus and mouse/human immunoglobulin, and mRNA splicing and polyadenylation regions can be the native chromosomal immunoglobulin sequences.

In some embodiments, for expression of cDNA genes in rodent cells, the transcriptional promoter is a viral LTR sequence, the transcriptional promoter enhancers are either or both the mouse immunoglobulin heavy chain enhancer and the viral LTR enhancer, the splice region contains an intron of greater than 31 bp, and the polyadenylation and transcription termination regions are derived from the native chromosomal sequence corresponding to the immunoglobulin chain being synthesized. In other embodiments, cDNA sequences encoding other proteins are combined with the above-recited expression elements to achieve expression of the proteins in mammalian cells.

Each fused gene is assembled in, or inserted into, an expression vector. Recipient cells capable of expressing the chimeric immunoglobulin chain gene product are then transfected singly with an antibody, antigen-binding portion thereof, or chimeric H or chimeric L chain-encoding gene, or are co-transfected with a chimeric H and a chimeric L chain gene. The transfected recipient cells are cultured under conditions that permit expression of the incorporated genes and the expressed immunoglobulin chains or intact antibodies or fragments are recovered from the culture.

In some embodiments, the fused genes encoding the antibody, antigen-binding fragment thereof, or chimeric H and L chains, or portions thereof are assembled in separate expression vectors that are then used to co-transfect a recipient cell. Each vector can contain two selectable genes, a first selectable gene designed for selection in a bacterial system and a second selectable gene designed for selection in a eukaryotic system, wherein each vector has a different pair of genes. This strategy results in vectors which first direct the production, and permit amplification, of the fused genes in a bacterial system. The genes so produced and amplified in a bacterial host are subsequently used to co-transfect a eukaryotic cell, and allow selection of a co-transfected cell carrying the desired transfected genes. Non-limiting examples of selectable genes for use in a bacterial system are the gene that confers resistance to ampicillin and the gene that confers resistance to chloramphenicol. Selectable genes for use in eukaryotic transfectants include the xanthine guanine phosphoribosyl transferase gene (designated gpt) and the phosphotransferase gene from Tn5 (designated neo). Alternatively the fused genes encoding chimeric H and L chains can be assembled on the same expression vector.

For transfection of the expression vectors and production of the chimeric, humanized, or composite human antibodies described herein, the recipient cell line can be a myeloma cell. Myeloma cells can synthesize, assemble and secrete immunoglobulins encoded by transfected immunoglobulin genes and possess the mechanism for glycosylation of the immunoglobulin. For example, in some embodiments, the recipient cell is the recombinant Ig-producing myeloma cell SP2/0 (ATCC #CRL 8287). SP2/0 cells produce only immunoglobulin encoded by the transfected genes. Myeloma cells can be grown in culture or in the peritoneal cavity of a mouse, where secreted immunoglobulin can be obtained from ascites fluid. Other suitable recipient cells include lymphoid cells such as B lymphocytes of human or non-human origin, hybridoma cells of human or non-human origin, or interspecies heterohybridoma cells.

An expression vector carrying a chimeric, humanized, or composite human antibody construct, antibody, or antigen-binding portion thereof as described herein can be introduced into an appropriate host cell by any of a variety of suitable means, including such biochemical means as transformation, transfection, conjugation, protoplast fusion, calcium phosphate-precipitation, and application with polycations such as diethylaminoethyl (DEAE) dextran, and such mechanical means as electroporation, direct microinjection, and microprojectile bombardment. Johnston et al., 240 Science 1538 (1988), as known to one of ordinary skill in the art.

Yeast provides certain advantages over bacteria for the production of immunoglobulin H and L chains. Yeasts carry out post-translational peptide modifications including glycosylation. A number of recombinant DNA strategies exist that utilize strong promoter sequences and high copy number plasmids which can be used for production of the desired proteins in yeast. Yeast recognizes leader sequences of cloned mammalian gene products and secretes peptides bearing leader sequences (i.e., pre-peptides). Hitzman et al., 11th Intl. Conf. Yeast, Genetics & Molec. Biol. (Montpelier, France, 1982).

Yeast gene expression systems can be routinely evaluated for the levels of production, secretion and the stability of antibodies, and assembled chimeric, humanized, or composite human antibodies, portions and regions thereof. Any of a series of yeast gene expression systems incorporating promoter and termination elements from the actively expressed genes coding for glycolytic enzymes produced in large quantities when yeasts are grown in media rich in glucose can be utilized. Known glycolytic genes can also provide very efficient transcription control signals. For example, the promoter and terminator signals of the phosphoglycerate kinase (PGK) gene can be utilized. A number of approaches can be taken for evaluating optimal expression plasmids for the expression of cloned immunoglobulin cDNAs in yeast. See II DNA Cloning 45, (Glover, ed., IRL Press, 1985) and e.g., U.S. Publication No. US 2006/0270045 A1.

Bacterial strains can also be utilized as hosts for the production of the antibody molecules or peptides described herein, E. coli K12 strains such as E. coli W3110 (ATCC 27325), Bacillus species, enterobacteria such as Salmonella typhimurium or Serratia marcescens, and various Pseudomonas species can be used. Plasmid vectors containing replicon and control sequences which are derived from species compatible with a host cell are used in connection with these bacterial hosts. The vector carries a replication site, as well as specific genes which are capable of providing phenotypic selection in transformed cells. A number of approaches can be taken for evaluating the expression plasmids for the production of chimeric, humanized, or composite humanized antibodies and fragments thereof encoded by the cloned immunoglobulin cDNAs or CDRs in bacteria (see Glover, 1985; Ausubel, 1987, 1993; Sambrook, 1989; Colligan, 1992-1996).

Host mammalian cells can be grown in vitro or in vivo. Mammalian cells provide post-translational modifications to immunoglobulin protein molecules including leader peptide removal, folding and assembly of H and L chains, glycosylation of the antibody molecules, and secretion of functional antibody protein.

Mammalian cells which can be useful as hosts for the production of antibody proteins, in addition to the cells of lymphoid origin described above, include cells of fibroblast origin, such as Vero (ATCC CRL 81) or CHO-K1 (ATCC CRL 61) cells. Exemplary eukaryotic cells that can be used to express polypeptides include, but are not limited to, COS cells, including COS 7 cells; 293 cells, including 293-6E cells; CHO cells, including CHO—S and DG44 cells; PER.C6™ cells (Crucell); and NSO cells. In some embodiments, a particular eukaryotic host cell is selected based on its ability to make desired post-translational modifications to the heavy chains and/or light chains. For example, in some embodiments, CHO cells produce polypeptides that have a higher level of sialylation than the same polypeptide produced in 293 cells.

In some embodiments, one or more antibodies or antigen-binding portions thereof as described herein can be produced in vivo in an animal that has been engineered or transfected with one or more nucleic acid molecules encoding the polypeptides, according to any suitable method.

In some embodiments, an antibody or antigen-binding portion thereof as described herein is produced in a cell-free system. Nonlimiting exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

Many vector systems are available for the expression of cloned H and L chain genes in mammalian cells (see Glover, 1985). Different approaches can be followed to obtain complete H₂L₂ antibodies. As discussed above, it is possible to co-express H and L chains in the same cells to achieve intracellular association and linkage of H and L chains into complete tetrameric H₂L₂ antibodies or antigen-binding portions thereof. The co-expression can occur by using either the same or different plasmids in the same host. Genes for both H and L chains or portions thereof can be placed into the same plasmid, which is then transfected into cells, thereby selecting directly for cells that express both chains. Alternatively, cells can be transfected first with a plasmid encoding one chain, for example the L chain, followed by transfection of the resulting cell line with an H chain plasmid containing a second selectable marker. Cell lines producing antibodies, antigen-binding portions thereof and/or H₂L₂ molecules via either route could be transfected with plasmids encoding additional copies of peptides, H, L, or H plus L chains in conjunction with additional selectable markers to generate cell lines with enhanced properties, such as higher production of assembled H₂L₂ antibody molecules or enhanced stability of the transfected cell lines.

Additionally, plants have emerged as a convenient, safe and economical alternative main-stream expression systems for recombinant antibody production, which are based on large scale culture of microbes or animal cells. Antibodies can be expressed in plant cell culture, or plants grown conventionally. The expression in plants may be systemic, limited to sub-cellular plastids, or limited to seeds (endosperms). See, e.g., U.S. Patent Pub. No. 2003/0167531; U.S. Pat. No. 6,080,560; U.S. Pat. No. 6,512,162; WO 0129242. Several plant-derived antibodies have reached advanced stages of development, including clinical trials (see, e.g., Biolex, NC).

In some aspects, provided herein are methods and systems for the production of a humanized antibody, which is prepared by a process which comprises maintaining a host transformed with a first expression vector which encodes the light chain of the humanized antibody and with a second expression vector which encodes the heavy chain of the humanized antibody under such conditions that each chain is expressed and isolating the humanized antibody formed by assembly of the thus-expressed chains. The first and second expression vectors can be the same vector. Also provided herein are DNA sequences encoding the light chain or the heavy chain of the humanized antibody; an expression vector which incorporates a said DNA sequence; and a host transformed with a said expression vector.

Generating a humanized antibody from the sequences and information provided herein can be practiced by those of ordinary skill in the art without undue experimentation. In one approach, there are four general steps employed to humanize a monoclonal antibody, see, e.g., U.S. Pat. No. 5,585,089; U.S. Pat. No. 6,835,823; U.S. Pat. No. 6,824,989. These are: (1) determining the nucleotide and predicted amino acid sequence of the starting antibody light and heavy variable domains; (2) designing the humanized antibody, i.e., deciding which antibody framework region to use during the humanizing process; (3) the actual humanizing methodologies/techniques; and (4) the transfection and expression of the humanized antibody.

Usually the CDR regions in humanized antibodies and human antibody variants are substantially identical, and more usually, identical to the corresponding CDR regions in the mouse or human antibody from which they were derived. Although not usually desirable, it is sometimes possible to make one or more conservative amino acid substitutions of CDR residues without appreciably affecting the binding affinity of the resulting humanized immunoglobulin or human antibody variant. Occasionally, substitutions of CDR regions can enhance binding affinity.

In addition, techniques developed for the production of “chimeric antibodies” (see Morrison et al., Proc. Natl. Acad. Sci. 81:851-855 (1984); Neuberger et al., Nature 312:604-608 (1984); Takeda et al., Nature 314:452-454 (1985); which are incorporated by reference herein in their entireties) by splicing genes from a mouse, or other species, antibody molecule of appropriate antigen specificity together with genes from a human antibody molecule of appropriate biological activity can be used. A chimeric antibody is a molecule in which different portions are derived from different animal species, such as those having a variable region derived from a murine monoclonal antibody and a human immunoglobulin constant region, e.g., humanized antibodies.

The variable segments of chimeric antibodies are typically linked to at least a portion of an immunoglobulin constant region (Fc), typically that of a human immunoglobulin. Human constant region DNA sequences can be isolated in accordance with well-known procedures from a variety of human cells, such as immortalized B-cells (WO 87/02671; which is incorporated by reference herein in its entirety). The antibody can contain both light chain and heavy chain constant regions. The heavy chain constant region can include CH1, hinge, CH2, CH3, and, sometimes, CH4 regions. For therapeutic purposes, the CH2 domain can be deleted or omitted.

Alternatively, techniques described for the production of single chain antibodies (see, e.g. U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989); which are incorporated by reference herein in their entireties) can be adapted to produce single chain antibodies. Single chain antibodies are formed by linking the heavy and light chain fragments of the Fv region via an amino acid bridge, resulting in a single chain polypeptide. Techniques for the assembly of functional Fv fragments in E. coli can also be used (see, e.g. Skerra et al., Science 242:1038-1041 (1988); which is incorporated by reference herein in its entirety).

Chimeric, humanized and human antibodies are typically produced by recombinant expression. Recombinant polynucleotide constructs typically include an expression control sequence operably linked to the coding sequences of antibody chains, including naturally-associated or heterologous promoter regions. Preferably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences, and the collection and purification of the cross-reacting antibodies. These expression vectors are typically replicable in the host organisms either as episomes or as an integral part of the host chromosomal DNA. Commonly, expression vectors contain selection markers, e.g., ampicillin-resistance or hygromycin-resistance, to permit detection of those cells transformed with the desired DNA sequences. E. coli is one prokaryotic host particularly useful for cloning the DNA sequences. Microbes, such as yeast are also useful for expression. Saccharomyces is a preferred yeast host, with suitable vectors having expression control sequences, an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization. Mammalian cells are a preferred host for expressing nucleotide segments encoding immunoglobulins or fragments thereof. See Winnacker, From Genes to Clones, (VCH Publishers, N Y, 1987), which is incorporated herein by reference in its entirety. A number of suitable host cell lines capable of secreting intact heterologous proteins have been developed in the art, and include CHO cell lines, various COS cell lines, HeLa cells, L cells and multiple myeloma cell lines. Expression vectors for these cells can include expression control sequences, such as an origin of replication, a promoter, an enhancer (Queen et al., “Cell-type Specific Regulation of a Kappa Immunoglobulin Gene by Promoter and Enhancer Elements,” Immunol Rev 89:49 (1986), incorporated herein by reference in its entirety), and necessary processing information sites, such as ribosome binding sites, RNA splice sites, polyadenylation sites, and transcriptional terminator sequences. Preferred expression control sequences are promoters substantially similar to a region of the endogenous genes, cytomegalovirus, SV40, adenovirus, bovine papillomavirus, and the like. See Co et al., “Chimeric and Humanized Antibodies with Specificity for the CD33 Antigen,” J Immunol 148:1149 (1992), which is incorporated herein by reference in its entirety. Alternatively, antibody coding sequences can be incorporated in transgenes for introduction into the genome of a transgenic animal and subsequent expression in the milk of the transgenic animal (e.g., according to methods described in U.S. Pat. No. 5,741,957, U.S. Pat. No. 5,304,489, U.S. Pat. No. 5,849,992, all incorporated by reference herein in their entireties). Suitable transgenes include coding sequences for light and/or heavy chains in operable linkage with a promoter and enhancer from a mammary gland specific gene, such as casein or beta lactoglobulin. The vectors containing the DNA segments of interest can be transferred into the host cell by well-known methods, depending on the type of cellular host. For example, calcium chloride transfection is commonly utilized for prokaryotic cells, whereas calcium phosphate treatment, electroporation, lipofection, biolistics or viral-based transfection can be used for other cellular hosts. Other methods used to transform mammalian cells include the use of polybrene, protoplast fusion, liposomes, electroporation, and microinjection (see generally, Sambrook et al., supra, which is herein incorporated by reference in is entirety). For production of transgenic animals, transgenes can be microinjected into fertilized oocytes, or can be incorporated into the genome of embryonic stem cells, and the nuclei of such cells transferred into enucleated oocytes. Once expressed, antibodies can be purified according to standard procedures of the art, including HPLC purification, column chromatography, gel electrophoresis and the like (see generally, Scopes, Protein Purification (Springer-Verlag, N Y, 1982), which is incorporated herein by reference in its entirety).

Once expressed, the whole antibodies, their dimers, individual light and heavy chains, or other immunoglobulin forms of the present invention can be recovered and purified by known techniques, e.g., immunoabsorption or immunoaffinity chromatography, chromatographic methods such as HPLC (high performance liquid chromatography), ammonium sulfate precipitation, gel electrophoresis, or any combination of these. See generally, Scopes, PROTEIN PURIF. (Springer-Verlag, N Y, 1982). Substantially pure immunoglobulins of at least about 90% to 95% homogeneity are advantageous, as are those with 98% to 99% or more homogeneity, particularly for pharmaceutical uses. Once purified, partially or to homogeneity as desired, a humanized or composite human antibody can then be used therapeutically or in developing and performing assay procedures, immunofluorescent stainings, and the like. See generally, Vols. I & II Immunol Meth. (Lefkovits & Pernis, eds., Acad. Press, N Y, 1979 and 1981).

In some embodiments, the technology described herein relates to a nucleic acid encoding an antibody or antigen-binding portion thereof as described herein. As used herein, the term “nucleic acid” or “nucleic acid sequence” refers to a polymeric molecule incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA.

In some embodiments, a nucleic acid encoding an antibody or antigen-binding portion thereof as described herein is comprised by a vector. In some of the aspects described herein, a nucleic acid sequence encoding an antibody or antigen-binding portion thereof as described herein, or any module thereof, is operably linked to a vector. The term “vector”, as used herein, refers to a nucleic acid construct designed for delivery to a host cell or for transfer between different host cells. As used herein, a vector can be viral or non-viral. The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc.

As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in human cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “viral vector” refers to a nucleic acid vector construct that includes at least one element of viral origin and has the capacity to be packaged into a viral vector particle. The viral vector can contain the nucleic acid encoding an antibody or antigen-binding portion thereof as described herein in place of non-essential viral genes. The vector and/or particle may be utilized for the purpose of transferring any nucleic acids into cells either in vitro or in vivo. Numerous forms of viral vectors are known in the art.

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. In some embodiments, the vector is episomal. The use of a suitable episomal vector provides a means of maintaining the nucleotide of interest in the subject in high copy number extra chromosomal DNA thereby eliminating potential effects of chromosomal integration.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable nucleic acid molecules are DNA, including genomic DNA or cDNA. Other suitable nucleic acid molecules are RNA, including mRNA.

Aptamers are short synthetic single-stranded oligonucleotides that specifically bind to various molecular targets such as small molecules, proteins, nucleic acids, and even cells and tissues. These small nucleic acid molecules can form secondary and tertiary structures capable of specifically binding proteins or other cellular targets, and are essentially a chemical equivalent of antibodies. Aptamers are highly specific, relatively small in size, and non-immunogenic. Aptamers are generally selected from a biopanning method known as SELEX (Systematic Evolution of Ligands by Exponential enrichment) (Ellington et al. Nature. 1990; 346(6287):818-822; Tuerk et al., Science. 1990; 249(4968):505-510; Ni et al., Curr Med Chem. 2011; 18(27):4206-14; which are incorporated by reference herein in their entireties). Methods of generating an apatmer for any given target are well known in the art. Preclinical studies using, e.g. aptamer-siRNA chimeras and aptamer targeted nanoparticle therapeutics have been very successful in mouse models of cancer and HIV (Ni et al., Curr Med Chem. 2011; 18(27):4206-14).

In some embodiments, a nucleic acid, e.g. an mRNA encoding a Mfsd2A polypeptide can be a modified mRNA. Modifications that improve the half-life, translation efficiency, and/or efficacy of a nucleic are known in the art. Non-limiting examples of such modifications can include the inclusion of a nucleoside selected from the group consisting of pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, 4-methoxy-2-thio-pseudouridine, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, 4-methoxy-1-methyl-pseudoisocytidine, 2-aminopurine, 2,6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, 2-methoxy-adenine, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, l-methyl-6-thio-guanosine, N2-methyl-6-thio-guanosine, N2,N2-dimethyl-6-thio-guanosine, 5-methylcytidine (5mC), N6-methyladenosine (m6A), 3,2′-0-dimethyluridine (m4U), 2-thiouridine (s2U), 2′ fluorouridine, pseudouridine, 2′-0-methyluridine (Um), 2′deoxy uridine (2′ dU), 4-thiouridine (s4U), 5-methyluridine (m5U), 2′-0-methyladenosine (m6A), N6,2′-0-dimethyladenosine (m6Am), N6,N6,2′-0-trimethyladenosine (m62Am), 2′-0-methylcytidine (Cm), 7-methylguanosine (m7G), 2′-0-methylguanosine (Gm), N2,7-dimethylguanosine (m2,7G), and N2,N2,7-trimethylguanosine (m2,2,7G). Additional non-limiting modifications can include, chemical modifications of a nucleotide wherein the nucleotide has altered binding to major groove interacting partners, a modification located on the major groove face of the nucleobase, and wherein the chemical modifications can include replacing or substituting an atom of a pyrimidine nucleobase with an amine, an SH, an alkyl (e.g., methyl or ethyl), or a halo (e.g., chloro or fluoro), chemical modifications located on the sugar moiety of the nucleotide, chemical modifications located on the phosphate backbone of the nucleic acid, chemical modifications that alter the electrochemistry on the major groove face of the nucleic acid, and chemical modifications wherein the nucleotide reduces the cellular innate immune response, as compared to the cellular innate immune induced by a corresponding unmodified nucleic acid. Approximately one hundred different nucleoside modifications have been identified in RNA (Rozenski, J, Crain, P, and McCloskey, J. (1999). The RNA Modification Database: 1999 update. Nucl Acids Res 27: 196-197). Modified mRNAs and methods of producing them are described, e.g. in US Patent Publications US2013/0115272, US2013/0115272, and US2013/0123481 and International Patent Publications PCT/US11/32679, PCT/US2012/058519, PCT/US12/054574, and PCT/US12/05456; each of which is incorporated by reference herein in its entirety.

Inhibitors of the expression of a given gene can be an inhibitory nucleic acid. In some embodiments, the inhibitory nucleic acid is an inhibitory RNA (iRNA). Double-stranded RNA molecules (dsRNA) have been shown to block gene expression in a highly conserved regulatory mechanism known as RNA interference (RNAi). The inhibitory nucleic acids described herein can include an RNA strand (the antisense strand) having a region which is 30 nucleotides or less in length, i.e., 15-30 nucleotides in length, generally 19-24 nucleotides in length, which region is substantially complementary to at least part the targeted mRNA transcript. The use of these iRNAs enables the targeted degradation of mRNA transcripts, resulting in decreased expression and/or activity of the target.

As used herein, the term “iRNA” refers to an agent that contains RNA as that term is defined herein, and which mediates the targeted cleavage of an RNA transcript via an RNA-induced silencing complex (RISC) pathway. In one embodiment, an iRNA as described herein effects inhibition of the expression and/or activity of Mfsd2A. In certain embodiments, contacting a cell with the inhibitor (e.g. an iRNA) results in a decrease in the target mRNA level in a cell by at least about 5%, about 10%, about 20%, about 30%, about 40%, about 50%, about 60%, about 70%, about 80%, about 90%, about 95%, about 99%, up to and including 100% of the target mRNA level found in the cell without the presence of the iRNA.

In some embodiments, the iRNA can be a dsRNA. A dsRNA includes two RNA strands that are sufficiently complementary to hybridize to form a duplex structure under conditions in which the dsRNA will be used. One strand of a dsRNA (the antisense strand) includes a region of complementarity that is substantially complementary, and generally fully complementary, to a target sequence. The target sequence can be derived from the sequence of an mRNA formed during the expression of the target. The other strand (the sense strand) includes a region that is complementary to the antisense strand, such that the two strands hybridize and form a duplex structure when combined under suitable conditions. Generally, the duplex structure is between 15 and 30 inclusive, more generally between 18 and 25 inclusive, yet more generally between 19 and 24 inclusive, and most generally between 19 and 21 base pairs in length, inclusive. Similarly, the region of complementarity to the target sequence is between 15 and 30 inclusive, more generally between 18 and 25 inclusive, yet more generally between 19 and 24 inclusive, and most generally between 19 and 21 nucleotides in length, inclusive. In some embodiments, the dsRNA is between 15 and 20 nucleotides in length, inclusive, and in other embodiments, the dsRNA is between 25 and 30 nucleotides in length, inclusive. As the ordinarily skilled person will recognize, the targeted region of an RNA targeted for cleavage will most often be part of a larger RNA molecule, often an mRNA molecule. Where relevant, a “part” of an mRNA target is a contiguous sequence of an mRNA target of sufficient length to be a substrate for RNAi-directed cleavage (i.e., cleavage through a RISC pathway). dsRNAs having duplexes as short as 9 base pairs can, under some circumstances, mediate RNAi-directed RNA cleavage. Most often a target will be at least 15 nucleotides in length, preferably 15-30 nucleotides in length.

In yet another embodiment, the RNA of an iRNA, e.g., a dsRNA, is chemically modified to enhance stability or other beneficial characteristics. The nucleic acids featured in the invention may be synthesized and/or modified by methods well established in the art, such as those described in “Current protocols in nucleic acid chemistry,” Beaucage, S. L. et al. (Edrs.), John Wiley & Sons, Inc., New York, N.Y., USA, which is hereby incorporated herein by reference. Modifications include, for example, (a) end modifications, e.g., 5′ end modifications (phosphorylation, conjugation, inverted linkages, etc.) 3′ end modifications (conjugation, DNA nucleotides, inverted linkages, etc.), (b) base modifications, e.g., replacement with stabilizing bases, destabilizing bases, or bases that base pair with an expanded repertoire of partners, removal of bases (abasic nucleotides), or conjugated bases, (c) sugar modifications (e.g., at the 2′ position or 4′ position) or replacement of the sugar, as well as (d) backbone modifications, including modification or replacement of the phosphodiester linkages. Specific examples of RNA compounds useful in the embodiments described herein include, but are not limited to RNAs containing modified backbones or no natural internucleoside linkages. RNAs having modified backbones include, among others, those that do not have a phosphorus atom in the backbone. For the purposes of this specification, and as sometimes referenced in the art, modified RNAs that do not have a phosphorus atom in their internucleoside backbone can also be considered to be oligonucleosides. In particular embodiments, the modified RNA will have a phosphorus atom in its internucleoside backbone.

Modified RNA backbones can include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those) having inverted polarity wherein the adjacent pairs of nucleoside units are linked 3′-5′ to 5′-3′ or 2′-5′ to 5′-2′. Various salts, mixed salts and free acid forms are also included. Representative U.S. patents that teach the preparation of the above phosphorus-containing linkages include, but are not limited to, U.S. Pat. Nos. 3,687,808; 4,469,863; 4,476,301; 5,023,243; 5,177,195; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,316; 5,550,111; 5,563,253; 5,571,799; 5,587,361; 5,625,050; 6,028,188; 6,124,445; 6,160,109; 6,169,170; 6,172,209; 6,239,265; 6,277,603; 6,326,199; 6,346,614; 6,444,423; 6,531,590; 6,534,639; 6,608,035; 6,683,167; 6,858,715; 6,867,294; 6,878,805; 7,015,315; 7,041,816; 7,273,933; 7,321,029; and U.S. Pat. No. RE39464, each of which is herein incorporated by reference

Modified RNA backbones that do not include a phosphorus atom therein have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts. Representative U.S. patents that teach the preparation of the above oligonucleosides include, but are not limited to, U.S. Pat. Nos. 5,034,506; 5,166,315; 5,185,444; 5,214,134; 5,216,141; 5,235,033; 5,64,562; 5,264,564; 5,405,938; 5,434,257; 5,466,677; 5,470,967; 5,489,677; 5,541,307; 5,561,225; 5,596,086; 5,602,240; 5,608,046; 5,610,289; 5,618,704; 5,623,070; 5,663,312; 5,633,360; 5,677,437; and, 5,677,439, each of which is herein incorporated by reference.

In other RNA mimetics suitable or contemplated for use in iRNAs, both the sugar and the internucleoside linkage, i.e., the backbone, of the nucleotide units are replaced with novel groups. The base units are maintained for hybridization with an appropriate nucleic acid target compound. One such oligomeric compound, an RNA mimetic that has been shown to have excellent hybridization properties, is referred to as a peptide nucleic acid (PNA). In PNA compounds, the sugar backbone of an RNA is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082; 5,714,331; and 5,719,262, each of which is herein incorporated by reference. Further teaching of PNA compounds can be found, for example, in Nielsen et al., Science, 1991, 254, 1497-1500.

Some embodiments featured in the invention include RNAs with phosphorothioate backbones and oligonucleosides with heteroatom backbones, and in particular —CH₂—NH—CH₂—, —CH₂—N(CH₃)—O—CH₂— [known as a methylene (methylimino) or MMI backbone], —CH₂—O—N(CH₃)—CH₂—, —CH₂—N(CH₃)—N(CH₃)—CH₂— and —N(CH₃)—CH₂—CH₂— [wherein the native phosphodiester backbone is represented as —O—P—O—CH₂—] of the above-referenced U.S. Pat. No. 5,489,677, and the amide backbones of the above-referenced U.S. Pat. No. 5,602,240. In some embodiments, the RNAs featured herein have morpholino backbone structures of the above-referenced U.S. Pat. No. 5,034,506.

Modified RNAs can also contain one or more substituted sugar moieties. The iRNAs, e.g., dsRNAs, featured herein can include one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Exemplary suitable modifications include O[(CH₂)ₙO]ₘCH₃, O(CH₂).ₙOCH₃, O(CH₂)ₙNH₂, O(CH₂)ₙCH₃, O(CH₂)ₙONH₂, and O(CH₂)ₙON[(CH₂)ₙCH₃)]₂, where n and m are from 1 to about 10. In other embodiments, dsRNAs include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an iRNA, or a group for improving the pharmacodynamic properties of an iRNA, and other substituents having similar properties. In some embodiments, the modification includes a 2′-methoxyethoxy (2′-O—CH₂CH₂OCH₃, also known as 2′-O-(2-methoxyethyl) or 2′-MOE) (Martin et al., Helv. Chim. Acta, 1995, 78:486-504) i.e., an alkoxy-alkoxy group. Another exemplary modification is 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethylaminoethoxyethyl or 2′-DMAEOE), i.e., 2′-O—CH₂—O—CH₂—N(CH₂)₂, also described in examples herein below.

Other modifications include 2′-methoxy (2′-OCH₃), 2′-aminopropoxy (2′-OCH₂CH₂CH₂NH₂) and 2′-fluoro (2′-F). Similar modifications can also be made at other positions on the RNA of an iRNA, particularly the 3′ position of the sugar on the 3′ terminal nucleotide or in 2′-5′ linked dsRNAs and the 5′ position of 5′ terminal nucleotide. iRNAs may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; and 5,700,920, certain of which are commonly owned with the instant application, and each of which is herein incorporated by reference.

An iRNA can also include nucleobase (often referred to in the art simply as “base”) modifications or substitutions. As used herein, “unmodified” or “natural” nucleobases include the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl anal other 8-substituted adenines and guanines, 5-halo, particularly 5-bromo, 5-trifluoromethyl and other 5-substituted uracils and cytosines, 7-methylguanine and 7-methyladenine, 8-azaguanine and 8-azaadenine, 7-deazaguanine and 7-daazaadenine and 3-deazaguanine and 3-deazaadenine. Further nucleobases include those disclosed in U.S. Pat. No. 3,687,808, those disclosed in Modified Nucleosides in Biochemistry, Biotechnology and Medicine, Herdewijn, P. ed. Wiley-VCH, 2008; those disclosed in The Concise Encyclopedia Of Polymer Science And Engineering, pages 858-859, Kroschwitz, J. L, ed. John Wiley & Sons, 1990, these disclosed by Englisch et al., Angewandte Chemie, International Edition, 1991, 30, 613, and those disclosed by Sanghvi, Y S., Chapter 15, dsRNA Research and Applications, pages 289-302, Crooke, S. T. and Lebleu, B., Ed., CRC Press, 1993. Certain of these nucleobases are particularly useful for increasing the binding affinity of the oligomeric compounds featured in the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. (Sanghvi, Y. S., Crooke, S. T. and Lebleu, B., Eds., dsRNA Research and Applications, CRC Press, Boca Raton, 1993, pp. 276-278) and are exemplary base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,30; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,681,941; 6,015,886; 6,147,200; 6,166,197; 6,222,025; 6,235,887; 6,380,368; 6,528,640; 6,639,062; 6,617,438; 7,045,610; 7,427,672; and 7,495,088, each of which is herein incorporated by reference, and U.S. Pat. No. 5,750,692, also herein incorporated by reference.

The RNA of an iRNA can also be modified to include one or more locked nucleic acids (LNA). A locked nucleic acid is a nucleotide having a modified ribose moiety in which the ribose moiety comprises an extra bridge connecting the 2′ and 4′ carbons. This structure effectively “locks” the ribose in the 3′-endo structural conformation. The addition of locked nucleic acids to siRNAs has been shown to increase siRNA stability in serum, and to reduce off-target effects (Elmen, J. et al., (2005) Nucleic Acids Research 33(1):439-447; Mook, O R. et al., (2007) Mol Canc Ther 6(3):833-843; Grunweller, A. et al., (2003) Nucleic Acids Research 31(12):3185-3193). Representative U.S. patents that teach the preparation of locked nucleic acid nucleotides include, but are not limited to, the following: U.S. Pat. Nos. 6,268,490; 6,670,461; 6,794,499; 6,998,484; 7,053,207; 7,084,125; and 7,399,845, each of which is herein incorporated by reference in its entirety.

Another modification of the RNA of an iRNA featured in the invention involves chemically linking to the RNA one or more ligands, moieties or conjugates that enhance the activity, cellular distribution, pharmacokinetic properties, or cellular uptake of the iRNA. Such moieties include but are not limited to lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-S-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-phosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim Biophys. Acta, 1995, 1264:229-237), or an octadecylamine or hexylamino-carbonyloxycholesterol moiety (Crooke et al., J. Pharmacol. Exp. Ther., 1996, 277:923-937).

In some embodiments, the foregoing RNA modifications can also be applied to nucleic acids which are not iRNAs, e.g. a nucleic acid encoding an Mfsd2A polypeptide.

Nucleic acid molecules described herein, e.g. a nucleic acid encoding an Mfsd2A polypeptide or an iRNA, are prepared by a variety of methods known in the art. These methods include, but are not limited to, PCR, ligation, and direct synthesis. A nucleic acid sequence as described herein can be recombined with vector DNA in accordance with conventional techniques, including blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases. Techniques for such manipulations are disclosed, e.g., by Maniatis et al., Molecular Cloning, Lab. Manual (Cold Spring Harbor Lab. Press, N Y, 1982 and 1989), and Ausubel, 1987, 1993, and can be used to construct nucleic acid sequences as described herein.

The term “vector” encompasses any genetic element that is capable of replication when associated with the proper control elements and that can transfer gene sequences to cells. A vector can include, but is not limited to, a cloning vector, an expression vector, a plasmid, phage, transposon, cosmid, chromosome, virus, virion, etc. These transgenes can be introduced as a linear construct, a circular plasmid, or a viral vector, which can be an integrating or non-integrating vector. The transgene can also be constructed to permit it to be inherited as an extrachromosomal plasmid (Gassmann, et al., Proc. Natl. Acad. Sci. USA (1995) 92:1292).

In one aspect, the technology described herein relates to an expression vector comprising a nucleic acid as described herein. Such vectors can be used, e.g. to transform a cell in order to produce the encoded polypeptide or nucleic acid. As used herein, the term “expression vector” refers to a vector that directs expression of an RNA or polypeptide from sequences linked to transcriptional regulatory sequences on the vector. The sequences expressed will often, but not necessarily, be heterologous to the cell. An expression vector may comprise additional elements, for example, the expression vector may have two replication systems, thus allowing it to be maintained in two organisms, for example in mammalian cells for expression and in a prokaryotic host for cloning and amplification. The term “expression” refers to the cellular processes involved in producing RNA and proteins and as appropriate, secreting proteins, including where applicable, but not limited to, for example, transcription, transcript processing, translation and protein folding, modification and processing. “Expression products” include RNA transcribed from a gene, and polypeptides obtained by translation of mRNA transcribed from a gene. The term “gene” means the nucleic acid sequence which is transcribed (DNA) to RNA in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

By “recombinant vector” is meant a vector that includes a heterologous nucleic acid sequence, or “transgene” that is capable of expression in vivo. It should be understood that the vectors described herein can, in some embodiments, be combined with other suitable compositions and therapies. Vectors useful for the delivery of a sequence encoding an isolated peptide as described herein can include one or more regulatory elements (e.g., promoter, enhancer, etc.) sufficient for expression of the transgene in the desired cell or tissue. The regulatory elements can be chosen to provide either constitutive or regulated/inducible expression. As used herein, the term “viral vector” refers to a nucleic acid vector construct that includes at least one element of viral origin and has the capacity to be packaged into a viral vector particle. The viral vector can contain the nucleic acid as described herein in place of non-essential viral genes. The vector and/or particle may be utilized for the purpose of transferring any nucleic acids into cells either in vitro or in vivo. Numerous forms of viral vectors are known in the art.

Examples of vectors useful in delivery of nucleic acids as described herein include plasmid vectors, non-viral plasmid vectors (e.g. see U.S. Pat. Nos. 6,413,942, 6,214,804, 5,580,859, 5,589,466, 5,763,270 and 5,693,622, all of which are incorporated herein by reference in their entireties); retroviruses (e.g. see U.S. Pat. No. 5,219,740; Miller and Rosman (1989) BioTechniques 7:980-90; Miller, A. D. (1990) Human Gene Therapy 1:5-14; Scarpa et al. (1991) Virology 180:849-52; Miller et al., Meth. Enzymol. 217:581-599 (1993); Burns et al. (1993) Proc. Natl. Acad. Sci. USA 90:8033-37; Boris-Lawrie and Temin (1993) Curr. Opin. Genet. Develop. 3:102-09. Boesen et al., Biotherapy 6:291-302 (1994); Clowes et al., J. Clin. Invest. 93:644-651 (1994); Kiem et al., Blood 83:1467-1473 (1994); Salmons and Gunzberg, Human Gene Therapy 4:129-141 (1993); and Grossman and Wilson, Curr. Opin. in Genetics and Devel. 3:110-114 (1993), the contents of each of which are herein incorporated by reference in their entireties); lentiviruses (e.g., see U.S. Pat. Nos. 6,143,520; 5,665,557; and 5,981,276, the contents of which are herein incorporated by reference in their entireties); adenovirus-based expression vectors (e.g., see Haj-Ahmad and Graham (1986) J. Virol. 57:267-74; Bett et al. (1993) J. Virol. 67:5911-21; Mittereder et al. (1994) Human Gene Therapy 5:717-29; Seth et al. (1994) J. Virol. 68:933-40; Barr et al. (1994) Gene Therapy 1:51-58; Berkner, K. L. (1988) BioTechniques 6:616-29; and Rich et al. (1993) Human Gene Therapy 4:461-76; Wu et al. (2001) Anesthes. 94:1119-32; Parks (2000) Clin. Genet. 58:1-11; Tsai et al. (2000) Curr. Opin. Mol. Ther. 2:515-23; and U.S. Pat. Nos. 6,048,551; 6,306,652 and 6,306,652, incorporated herein by reference in their entireties); Adeno-associated viruses (AAV) (e.g. see U.S. Pat. Nos. 5,139,941; 5,622,856; 5,139,941; 6,001,650; and 6,004,797, the contents of each of which are incorporated by reference herein in their entireties); and avipox vectors (e.g. see WO 91/12882; WO 89/03429; and WO 92/03545; which are incorporated by reference herein in their entireties).

Useful methods of transfection can include, but are not limited to electroporation, sonoporation, protoplast fusion, peptoid delivery, or microinjection. See, e.g., Sambrook et al (1989) Molecular Cloning, A Laboratory Manual, Cold Spring Harbor Laboratories, New York, for a discussion of techniques for transforming cells of interest; and Feigner, P. L. (1990) Advanced Drug Delivery Reviews 5:163-87, for a review of delivery systems useful for gene transfer. Exemplary methods of delivering DNA using electroporation are described in U.S. Pat. Nos. 6,132,419; 6,451,002, 6,418,341, 6,233,483, U.S. Patent Publication No. 2002/0146831, and International Publication No. WO/0045823, all of which are incorporated herein by reference in their entireties.

In some embodiments, the nucleic acid as described herein can be operatively linked to, e.g. a promoter or other transcriptional regulatory sequence. The term “operatively linked” includes having an appropriate start signal (e.g., ATG) in front of the polynucleotide sequence to be expressed, and maintaining the correct reading frame to permit expression of the polynucleotide sequence under the control of the expression control sequence, and production of the desired polypeptide encoded by the polynucleotide sequence. In some examples, transcription of a nucleic acid modulatory compound is under the control of a promoter sequence (or other transcriptional regulatory sequence) which controls the expression of the nucleic acid in a cell-type in which expression is intended. It will also be understood that the modulatory nucleic acid can be under the control of transcriptional regulatory sequences which are the same or which are different from those sequences which control transcription of the naturally-occurring form of a protein. In some instances the promoter sequence is recognized by the synthetic machinery of the cell, or introduced synthetic machinery, required for initiating transcription of a specific gene.

In some embodiments, the vector comprising a nucleic acid encoding an isolated polypeptide as described herein and/or a nucleic acid encoding an isolated polypeptide as described herein, can be present in a cell. The cell can be, e.g. a microbial cell or a mammalian cell. In some embodiments, the cell as described herein is cultured under conditions suitable for the expression of the gene product as described herein. Such conditions can include, but are not limited to, conditions under which the cell is capable of growth and/or polypeptide synthesis. Conditions may vary depending upon the species and strain of cell selected. Conditions for the culture of cells, e.g. prokaryotic and mammalian cells, are well known in the art. If the recombinant polypeptide is operatively linked to an inducible promoter, such conditions can include the presence of the suitable inducing molecule(s).

The term “agent” refers generally to any entity which is normally not present or not present at the levels being administered to a cell, tissue or subject. An agent can be selected from a group including but not limited to: polynucleotides; polypeptides; small molecules; and antibodies or antigen-binding fragments thereof. A polynucleotide can be RNA or DNA, and can be single or double stranded, and can be selected from a group including, for example, nucleic acids and nucleic acid analogues that encode a polypeptide. A polypeptide can be, but is not limited to, a naturally-occurring polypeptide, a mutated polypeptide or a fragment thereof that retains the function of interest. Further examples of agents include, but are not limited to a nucleic acid aptamer, peptide-nucleic acid (PNA), locked nucleic acid (LNA), small organic or inorganic molecules; saccharide; oligosaccharides; polysaccharides; biological macromolecules, peptidomimetics; nucleic acid analogs and derivatives; extracts made from biological materials such as bacteria, plants, fungi, or mammalian cells or tissues and naturally occurring or synthetic compositions. An agent can be applied to the media, where it contacts the cell and induces its effects. Alternatively, an agent can be intracellular as a result of introduction of a nucleic acid sequence encoding the agent into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. In certain embodiments the agent is a small molecule having a chemical moiety selected, for example, from unsubstituted or substituted alkyl, aromatic, or heterocyclyl moieties including macrolides, leptomycins and related natural products or analogues thereof. Agents can be known to have a desired activity and/or property, or can be selected from a library of diverse compounds. As used herein, the term “small molecule” can refer to compounds that are “natural product-like,” however, the term “small molecule” is not limited to “natural product-like” compounds. Rather, a small molecule is typically characterized in that it contains several carbon-carbon bonds, and has a molecular weight more than about 50, but less than about 5000 Daltons (5 kD). Preferably the small molecule has a molecular weight of less than 3 kD, still more preferably less than 2 kD, and most preferably less than 1 kD. In some cases it is preferred that a small molecule have a molecular mass equal to or less than 700 Daltons.

A “subject in need” of treatment for a particular condition can be a subject having that condition, diagnosed as having that condition, or at risk of developing that condition.

As used herein, the terms “treat” “treatment” “treating,” or “amelioration” refer to therapeutic treatments, wherein the object is to reverse, alleviate, ameliorate, inhibit, slow down or stop the progression or severity of a condition associated with a disease or disorder, e.g. a neurodenerative disease or other disease affecting the CNS. The term “treating” includes reducing or alleviating at least one adverse effect or symptom of a condition, disease or disorder associated with the CNS. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein the term “comprising” or “comprises” is used in reference to compositions, methods, and respective component(s) thereof, that are essential to the method or composition, yet open to the inclusion of unspecified elements, whether essential or not.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of elements that do not materially affect the basic and novel or functional characteristic(s) of that embodiment.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Definitions of common terms in cell biology and molecular biology can be found in “The Merck Manual of Diagnosis and Therapy”, 19th Edition, published by Merck Research Laboratories, 2006 (ISBN 0-911910-19-0); Robert S. Porter et al. (eds.), The Encyclopedia of Molecular Biology, published by Blackwell Science Ltd., 1994 (ISBN 0-632-02182-9); Benjamin Lewin, Genes X, published by Jones & Bartlett Publishing, 2009 (ISBN-10: 0763766321); Kendrew et al. (eds.), Molecular Biology and Biotechnology: a Comprehensive Desk Reference, published by VCH Publishers, Inc., 1995 (ISBN 1-56081-569-8) and Current Protocols in Protein Sciences 2009, Wiley Intersciences, Coligan et al., eds.

Unless otherwise stated, the present invention was performed using standard procedures, as described, for example in Sambrook et al., Molecular Cloning: A Laboratory Manual (3 ed.), Cold Spring Harbor Laboratory Press, Cold Spring Harbor, N.Y., USA (2001); Davis et al., Basic Methods in Molecular Biology, Elsevier Science Publishing, Inc., New York, USA (1995); or Methods in Enzymology: Guide to Molecular Cloning Techniques Vol. 152, S. L. Berger and A. R. Kimmel Eds., Academic Press Inc., San Diego, USA (1987); Current Protocols in Protein Science (CPPS) (John E. Coligan, et. al., ed., John Wiley and Sons, Inc.), Current Protocols in Cell Biology (CPCB) (Juan S. Bonifacino et. al. ed., John Wiley and Sons, Inc.), and Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998) which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A method of modulating the permeability of the blood-brain barrier in a subject, the method comprising:
   administering an inhibitor of a gene or gene expression product selected from the group consisting of:
      Mfsd2A; Slco1C1; Slc38A5; LRP8; Slc3A2; Slc7A5; Slc7A1; Slc6A6; IGFBP7; Glut1; Slc40A1; and Slc30A1
   to the subject, whereby the permeability of the blood-brain barrier is increased; or administering an agonist of a gene or gene expression product selected from the group consisting of:
      Mfsd2A; Slco1C1; Slc38A5; LRP8; Slc3A2; Slc7A5; Slc7A1; Slc6A6; IGFBP7; Glut1; Slc40A1; and Slc30A1
   to the subject, whereby the permeability of the blood-brain barrier is decreased.
2. A method of treatment, the method comprising
   administering an inhibitor of a gene or gene expression product selected from the group consisting of:
      Mfsd2A; Slco1C1; Slc38A5; LRP8; Slc3A2; Slc7A5; Slc7A1; Slc6A6; IGFBP7; Glut1; Slc40A1; and Slc30A1
   to a subject in need of increased permeability of the blood-brain barrier; or administering an agonist of a gene or gene expression product selected from the group consisting of:
      Mfsd2A; Slco1C1; Slc38A5; LRP8; Slc3A2; Slc7A5; Slc7A1; Slc6A6; IGFBP7; Glut1; Slc40A1; and Slc30A1
   to the subject in need of decreased permeability of the blood-brain barrier.
3. The method of any of paragraphs 1-2, wherein the inhibitor is selected from the group consisting of inhibitory antibodies and inhibitory nucleic acids.
4. The method of any of paragraphs 1-3, wherein the inhibitor is an inhibitor of Mfsd2A.
5. The method of paragraph 4, wherein the inhibitor of Mfsd2A is selected from the group consisting of:
   tunicamycin; tunicamycin analogs; inhibitory anti-Mfsd2A antibodies; and inhibitory nucleic acids.
6. The method of any of paragraphs 2-4, wherein the subject administered an inhibitor is in need of delivery of a central nervous system therapeutic agent to the central nervous system.
7. The method of paragraph 6, wherein the method further comprises administering a central nervous system therapeutic agent to the subject.
8. The method of any of paragraphs 2-7, wherein the subject in need of increased permeability of the blood-brain barrier is in need of treatment for a condition selected from the group consisting of:
   brain cancer; encephalitis; hydrocephalus; Parksinson's disease; neuropathic pain;
   and a condition treated by the administration of psychiatric drugs.
9. The method of any of paragraphs 1-2, wherein the agonist is a polypeptide or a nucleic acid encoding a polypeptide selected from the group consisting of:
   a Mfsd2A polypeptide; a Slco1C1 polypeptide; a Slc38A5 polypeptide; a LRP8 polypeptide; a Slc3A2 polypeptide; a Slc7A5 polypeptide; a Slc7A1 polypeptide; a Slc6A6 polypeptide; a IGFBP7 polypeptide; a Glut1 polypeptide; a Slc40A1 polypeptide; and a Slc30A1 polypeptide.
10. The method of paragraph 2 or 9, wherein the subject administered an agonist is in need of improved quality of tight junctions of the blood-brain barrier.
11. The method of any of paragraphs 2 and 9-10, wherein the subject in need of decreased permeability of the blood-brain barrier is in need of treatment for a condition selected from the group consisting of:
   a neurodegenerative disease; multiple sclerosis; Parkinson's disease; Huntington's disease; Pick's disease; ALS; dementia; stroke; and Alzheimer's disease.
12. A pharmaceutical composition comprising an inhibitor of a gene or gene expression product selected from the group consisting of:
   Mfsd2A; Slco1C1; Slc38A5; LRP8; Slc3A2; Slc7A5; Slc7A1; Slc6A6; IGFBP7; Glut1; Slc40A1; and Slc30A1
   and a pharmaceutically-acceptable carrier.
13. The composition of paragraph 12, wherein the inhibitor is selected from the group consisting of inhibitory antibodies and inhibitory nucleic acids.
14. The composition of any of paragraphs 12-13, wherein the inhibitor is an inhibitor of Mfsd2A.
15. The composition of paragraph 14, wherein the inhibitor of Mfsd2A is selected from the group consisting of:
   tunicamycin; tunicamycin analogs; inhibitory anti-Mfsd2A antibodies; and inhibitory nucleic acids.
16. The composition of any of paragraphs 12-15, further comprising a central nervous system therapeutic agent.
17. A pharmaceutical composition comprising an agonist of a gene or gene expression product selected from the group consisting of:
   Mfsd2A; Slco1C1; Slc38A5; LRP8; Slc3A2; Slc7A5; Slc7A1; Slc6A6; IGFBP7; Glut1; Slc40A1; and Slc30A1
   and a pharmaceutically-acceptable carrier.
18. The composition of paragraph 17, wherein the agonist is a polypeptide or a nucleic acid encoding a polypeptide selected from the group consisting of:
   a Mfsd2A polypeptide; a Slco1C1 polypeptide; a Slc38A5 polypeptide; a LRP8 polypeptide; a Slc3A2 polypeptide; a Slc7A5 polypeptide; a Slc7A1 polypeptide; a Slc6A6 polypeptide; a IGFBP7 polypeptide; a Glut1 polypeptide; a Slc40A1 polypeptide; and a Slc30A1 polypeptide.
19. A method for determining the permeability of the blood-brain barrier during development, the method comprising:
   injecting the liver of an embryo with a detectable agent while the embryo is connected to the maternal circulation via the umbilical cord;
   allowing the dye to circulate in the bloodstream;
   detecting a signal from the detectable agent in blood vessels within the brain and within brain tissue separated from the bloodstream by the blood-brain barrier.
20. The method of paragraph 19, wherein the agent is a fixable dye.
21. The method of any of paragraphs 19-20, wherein the total volume of the injection is less than or equal to 1 uL for a murine embryo of about 13.5 days age, less than or equal to 2 uL for a murine embryo of about 14.5 days of age, and less than or equal to 5 uL for a murine embryo of about 15 days of age or older.
22. The method of any of paragraphs 19-21, wherein the agent is allowed to circulate for from about 30 seconds to about 30 minutes.
23. The method of any of paragraphs 19-22, wherein the agent is allowed to circulate for about 3 minutes.
24. The method of any of paragraphs 19-23, wherein the agent is fixed by immersion fixation.
25. The method of any of paragraphs 19-24, wherein the agent is fluoro-Ruby-Dextran.
26. A method for identifying a modulator of the permeability of the blood-brain barrier during development, the method comprising:
   administering a candidate modulator agent to an embryo;
   injecting the liver of an embryo with a detectable agent while the embryo is connected to the maternal circulation via the umbilical cord;
   allowing the dye to circulate in the bloodstream;
   detecting a signal from the detectable agent in blood vessels within the brain and within brain tissue separated from the bloodstream by the blood-brain barrier;
   wherein the candidate modulator is determined to increase permeability of the blood-brain barrier if the ratio of signal detected in brain tissue:signal detected in the blood vessels within the brain is lower than a reference level; and
   wherein the candidate modulator is determined to decrease permeability of the blood-brain barrier if the ratio of signal detected in brain tissue:signal detected in the blood vessels within the brain is higher than a reference level.
27. The method of paragraph 26, wherein the agent is a fixable dye.
28. The method of any of paragraphs 26-27, wherein the total volume of the injection is less than or equal to 1 uL for a murine embryo of about 13.5 days age, less than or equal to 2 uL for a murine embryo of about 14.5 days of age, and less than or equal to 5 uL for a murine embryo of about 15 days of age or older.
29. The method of any of paragraphs 26-28, wherein the agent is allowed to circulate for from about 30 seconds to about 30 minutes.
30. The method of any of paragraphs 26-29, wherein the agent is allowed to circulate for about 3 minutes.
31. The method of any of paragraphs 26-30, wherein the agent is fixed by immersion fixation.
32. The method of any of paragraphs 26-31, wherein the agent is fluoro-Ruby-Dextran.
33. A method of treatment, the method comprising
   administering to a subject in need of a central nervous system therapeutic agent a composition comprising:
      a) an antibody reagent that binds to a polypeptide selected from the group consisting of:
      Mfsd2A; Slco1C1; Slc38A5; LRP8; Slc3A2; Slc7A5; Slc7A1; Slc6A6; IGFBP7; Glut1; Slc40A1; and Slc30A1; and
      b) a central nervous system therapeutic agent.
34. The method of paragraph 33, wherein the composition is a bi-specific antibody.
35. The method of any of paragraphs 33-34, wherein the subject is in need of treatment for a condition selected from the group consisting of:
   brain cancer; encephalitis; hydrocephalus; Parksinson's disease; neuropathic pain; a condition treated by the administration of psychiatric drugs; a neurodegenerative disease; multiple sclerosis; Huntington's disease; Pick's disease; ALS; dementia; stroke; and Alzheimer's disease.
36. A pharmaceutical composition comprising
   a) an antibody reagent that binds to a polypeptide selected from the group consisting of:
      Mfsd2A; Slco1C1; Slc38A5; LRP8; Slc3A2; Slc7A5; Slc7A1; Slc6A6; IGFBP7; Glut1; Slc40A1; and Slc30A1;
   b) a central nervous system therapeutic agent;
   and a pharmaceutically-acceptable carrier.
37. A method of treatment, the method comprising
   administering an agonist of a gene or gene expression product selected from the group consisting of:
      Mfsd2A; Slco1C1; Slc38A5; LRP8; Slc3A2; Slc7A5; Slc7A1; Slc6A6; IGFBP7; Glut1; Slc40A1; and Slc30A1
   to the subject in need of treatment for a retinal disease.
38. The method of paragraph 37, wherein the agonist is a polypeptide or a nucleic acid encoding a polypeptide selected from the group consisting of:
   a Mfsd2A polypeptide; a Slco1C1 polypeptide; a Slc38A5 polypeptide; a LRP8 polypeptide; a Slc3A2 polypeptide; a Slc7A5 polypeptide; a Slc7A1 polypeptide; a Slc6A6 polypeptide; a IGFBP7 polypeptide; a Glut1 polypeptide; a Slc40A1 polypeptide; and a Slc30A1 polypeptide.
39. The method of paragraph 37 or 38, wherein the subject administered an agonist is in need of improved quality of the retinal barrier.
40. The method of any of paragraphs 37-39, wherein the subject is in need of treatment for a condition selected from the group consisting of:
   Glaucoma; diabetic retinopathy; and age-related macular degeneration.
41. A method of modulating the permeability of a tissue membrane in a subject, the method comprising:
   administering an inhibitor of a gene or gene expression product selected from the group consisting of:
      Mfsd2A; Slco1C1; Slc38A5; LRP8; Slc3A2; Slc7A5; Slc7A1; Slc6A6; IGFBP7; Glut1; Slc40A1; and Slc30A1
   to the subject, whereby the permeability of the tissue membrane is increased; or administering an agonist of a gene or gene expression product selected from the group consisting of:
      Mfsd2A; Slco1C1; Slc38A5; LRP8; Slc3A2; Slc7A5; Slc7A1; Slc6A6; IGFBP7; Glut1; Slc40A1; and Slc30A1
   to the subject, whereby the permeability of the tissue membrane is decreased.
42. A method of treatment, the method comprising
   administering an inhibitor of a gene or gene expression product selected from the group consisting of:
      Mfsd2A; Slco1C1; Slc38A5; LRP8; Slc3A2; Slc7A5; Slc7A1; Slc6A6; IGFBP7; Glut1; Slc40A1; and Slc30A1
   to a subject in need of increased permeability of a tissue membrane; or
   administering an agonist of a gene or gene expression product selected from the group consisting of:
      Mfsd2A; Slco1C1; Slc38A5; LRP8; Slc3A2; Slc7A5; Slc7A1; Slc6A6; IGFBP7; Glut1; Slc40A1; and Slc30A1
   to the subject in need of decreased permeability of the tissue membrane.
43. The method of any of paragraphs 41-42, wherein the tissue membrane is selected from the group consisting of:
   a kidney membrane; a placental membrane; or a testes membrane.
44. The method of any of paragraphs 41-43, wherein the inhibitor is selected from the group consisting of inhibitory antibodies and inhibitory nucleic acids.
45. The method of any of paragraphs 41-44, wherein the inhibitor is an inhibitor of Mfsd2A.
46. The method of paragraph 45, wherein the inhibitor of Mfsd2A is selected from the group consisting of:
   tunicamycin; tunicamycin analogs; inhibitory anti-Mfsd2A antibodies; and inhibitory nucleic acids.
47. The method of any of paragraphs 41-43, wherein the agonist is a polypeptide or a nucleic acid encoding a polypeptide selected from the group consisting of:
   a Mfsd2A polypeptide; a Slco1C1 polypeptide; a Slc38A5 polypeptide; a LRP8 polypeptide; a Slc3A2 polypeptide; a Slc7A5 polypeptide; a Slc7A1 polypeptide; a Slc6A6 polypeptide; a IGFBP7 polypeptide; a Glut1 polypeptide; a Slc40A1 polypeptide; and a Slc30A1 polypeptide.
48. The method of any of paragraphs 41-43 and 47, wherein the subject in need of decreased permeability of the tissue membrane is in need of treatment for a condition selected from the group consisting of:
   proteinuremia.
49. An antibody reagent that binds specifically to Mfsd2A.
50. The antibody reagent of paragraph 49, wherein the antibody reagent is selected from the group consisting of:
   a monoclonal antibody; a humanized antibody; a human antibody; a murine antibody; an intrabody; a single chain antibody; and an antigen-binding antibody fragment.
51. The antibody reagent of any of paragraphs 49-50, wherein the antibody reagent can bind specifically to an epitope comprising the amino acid corresponding to a residue of SEQ ID NO: 3 selected from the group consisting of:
   residue 92 and residue 96.
52. The antibody reagent of any of paragraphs 49-50, wherein the antibody reagent can bind specifically to an epitope comprising the amino acids corresponding the residues of SEQ ID NO: 3 selected from the group consisting of:
   1-52; 31-39; 99-114; 175-191; 268-298; 355-360; 406-428; 494-533; 506-509; 74-77; 136-150; 214-246; 319-331; 382-384; and 448-472.
53. The antibody reagent of any of paragraphs 49-50, wherein the antibody reagent can bind specifically to an epitope comprising at least 4 amino acids of the amino acids corresponding the residues of SEQ ID NO: 3 selected from the group consisting of:
   1-52; 31-39; 99-114; 175-191; 268-298; 355-360; 406-428; 494-533; 506-509; 74-77; 136-150; 214-246; 319-331; 382-384; and 448-472.

### EXAMPLES

### Example 1

### MSFD2A is Critical for Embryonic Formation of a Functional Blood Brain Barrier

The function of the central nervous system (CNS) depends on a tightly controlled environment that provides the proper chemical composition for synaptic transmissions and is free of various toxins and pathogens. This environment is maintained by highly specialized blood vessels that physically seal the CNS and control substance influx/efflux, known as the ‘blood brain barrier’ (BBB)¹. On one hand, BBB breakdown has recently been shown to be involved in the initiation and perpetuation of some neurological diseases. On the other hand, an intact BBB is a major obstacle for drug delivery to the CNS. However, limited understanding of the molecular mechanisms that control the BBB formation has hampered the ability to manipulate the BBB during diseases. Described herein is a method permitting the evaluation of BBB functionality at early developmental stages. Using this method, a temporal and spatial development profile of BBB functionality was observed and for the first time, concrete evidence is provided demonstrating that the mouse BBB becomes fully functional as early as embryonic day 15.5 (E15.5). Guided by this temporal information, an unbiased approach was used to identify BBB specific genes at the time when the BBB is actively forming. As described herein, t major facilitator super family domain containing 2a (Mfsd2a) is selectively expressed in BBB-containing blood vessels in the CNS, but not in the non-BBB blood vessels of either circumventricular organs in the CNS or blood vessels from the rest of the body. Finally, genetic ablation of Mfsd2a resulted in leaky BBB both at E15.5 and postnatal stages, while vasculature network architecture developed normally. Therefore, MFSD2A is required in vivo for barrier-genesis but not for CNS angiogenesis.

The central nervous system (CNS) functions in a tightly controlled and stable environment. This is maintained by highly specialized blood vessels that physically seal the CNS and control substance influx/efflux, known as the ‘blood brain barrier’ (BBB)¹. A single layer of endothelial cells lining the CNS capillaries is thought to be the site of the BBB and specialized tight junctions of these cells were shown to be the physical seal between blood and brain^{2,3}. BBB selectivity is facilitated by an array of endothelial transporters responsible for the supply of nutrients and for the clearance of waste or toxins⁴. In concert with pericytes and astrocytes, the BBB protects the brain from various toxins and pathogens and provides the proper chemical composition for synaptic transmissions. Therefore, proper function of the CNS critically depends on BBB integrity. Indeed emerging evidence shows that BBB breakdown occurs in many neurodegenerative diseases prior to noticeable neuronal abnormalities. On the other hand, the BBB is also a major obstacle for drug delivery to the CNS, proximally 98% of small molecules and most large molecules/biologics can not freely pass through the BBB. Therefore, attempts have been made, both to “loosen” the BBB and to “re-seal” the BBB to treat various CNS disorders. However, in both cases, a limited understanding of BBB formation at the molecular level has hampered these efforts.

The BBB is a unique feature of CNS blood vessels compared to blood vessels in the rest of the body. However, when and how these endothelial cells of CNS blood vessels acquired this property is still debated. The prevailing view has been that embryonic and even newborn BBB is not yet functional and thus, leaky′. However, previous studies of embryonic and newborn BBB functionality were mainly performed by trans-cardiac dye/tracer injection. Such direct injection into the embryo blood circulation may dramatically affect blood pressure, causing fragile CNS capillaries to burst, resulting in an artificial leakiness phenotype. To circumvent this caveat, a new method allowing for the detection of BBB integrity during development was developed. This method was based on the well established adult BBB dye injection assay with special considerations of the injection site and volume to cater the nature of embryonic vasculature (Risau et al. 1986), (Ek C J et al. 2006) (Stern et al. 1929)¹⁴⁻¹⁶. For adult BBB functionality, typical dyes of different molecular size are acutely injected into the tail vein and the extent of leakiness is visualized in brain slices. The embryonic injection method described herein contains four major modifications: 1. Embryos are injected while attached to the maternal circulation via the umbilical cord, minimizing abrupt changes in blood flow. 2. Taking advantage of the fenestrated and very permeable liver vasculature, dye is injected into the embryonic liver where it is being taken into the circulation in a matter of seconds. 3. Dye volume is adjusted to a minimum that still allows the detection in all CNS capillaries after 3 minutes (high fluoresce intensity dyes enable use of small volume and facilitate detection at single capillary levels). 4. Traditional perfusion fixation was omitted, again to prevent damage to capillaries. Fixable dyes were used to allow reliable immobilization of the dye at the end of the circulation time by immersion fixation.

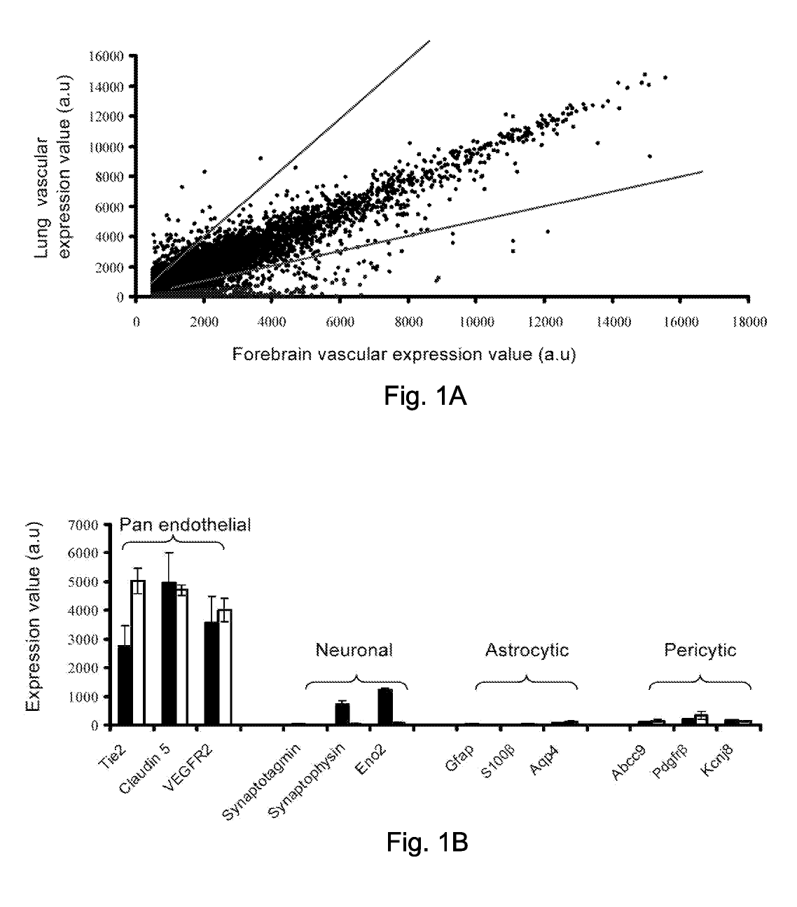

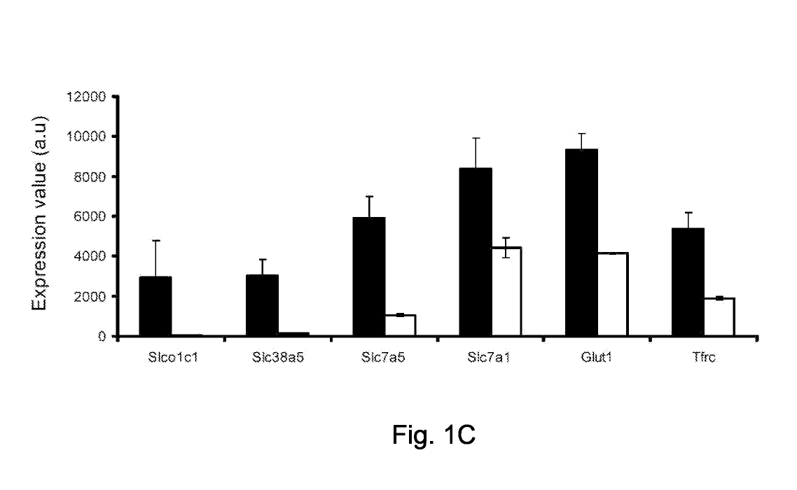

This new method was used to determine the precise timing of BBB formation in the developing mouse brain and observed a spatial and temporal pattern of functional barrier-genesis. The use of a lysine-fixable dye enables reliable co-labeling with vascular markers to allow visualization of both vessels and the injected dye. At least 6 embryos from each of 3 litters were used for each timepoint. BBB formation in the forebrain was focused upon. At embryonic day (E) 115, most of the 10 kDa dextran-dye leaked out of the capillaries and was taken up by non-vascular cells, mostly the surrounding neuro-progenitors. E14.5, most of the dye was located within the capillaries, but a diffused pattern could be detected outside of the vessels even though there was no visible dye uptake in individual neuro-progenitors any more. Finally, at E15.5 all the dye was confined in vessels with no detectible signal in brain parenchyma, similar to the adult functional BBB. These data demonstrate that after vessel ingression, the BBB gradually becomes functional as early as E15.5. Besides this temporal pattern, a spatial pattern of BBB functionality in different regions of the brain was observed. At E14.5, although forebrain vasculature does not yet exhibit a functional barrier, hind- and mid-brain vasculature is already capable of preventing 10 kDa dextran leakage (data not shown). Therefore, brain BBB formation exhibits a caudal to rostral spatial developmental pattern. Even within the forebrain, the latest barrier developing region within the brain, spatial differences were found (FIG. 1A); at E13.5, although most of the dye escapes the vessels in dorso-medial regions, most of the dextran was detected inside vessels in ventro-lateral regions. At E13.5 both diffuse tracer and neuro-progenitor cells stained with the injected tracer are apparent. Little tracer is detected inside the capillaries of dorsal regions while ventral regions capillaries are already less leaky with more tracer inside capillaries and less tracer in brain parenchyma (data not shown).

Similarly, the ventro-lateral regions are already fully functional at E14.5 while dorsal-medial regions are still leaky. At E14.5 BBB of ventral regions is already fully functional with all the injected tracer apparent inside capillaries and no detectible tracer in brain parenchyma. In contrast in dorsal regions diffuse tracer is still apparent in brain parenchyma (data not shown). Therefore, BBB formation exhibits a spatial pattern in the forebrain from ventral-lateral to dorsal-medial. This spatial pattern of development is reminiscent of other neurodevelopmental processes such as tangential migration path of inhibitory neuro-progenitors, deposit of extracellular matrix components and cortical plate expansion¹⁷⁻¹⁹.

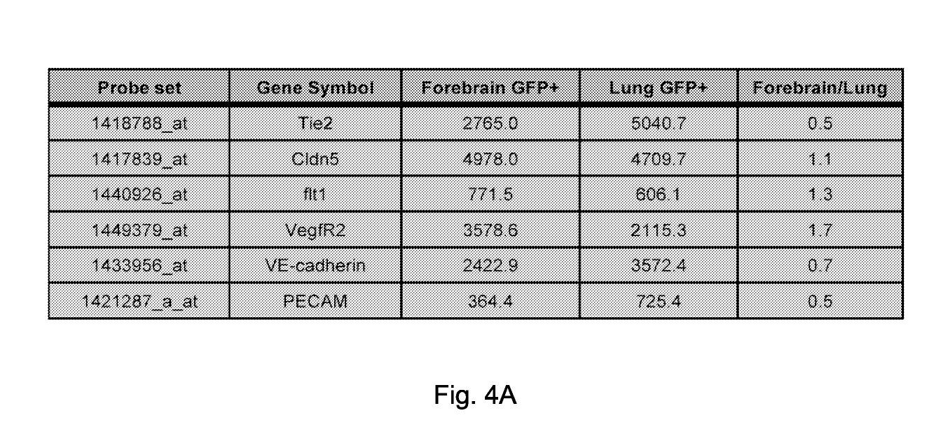

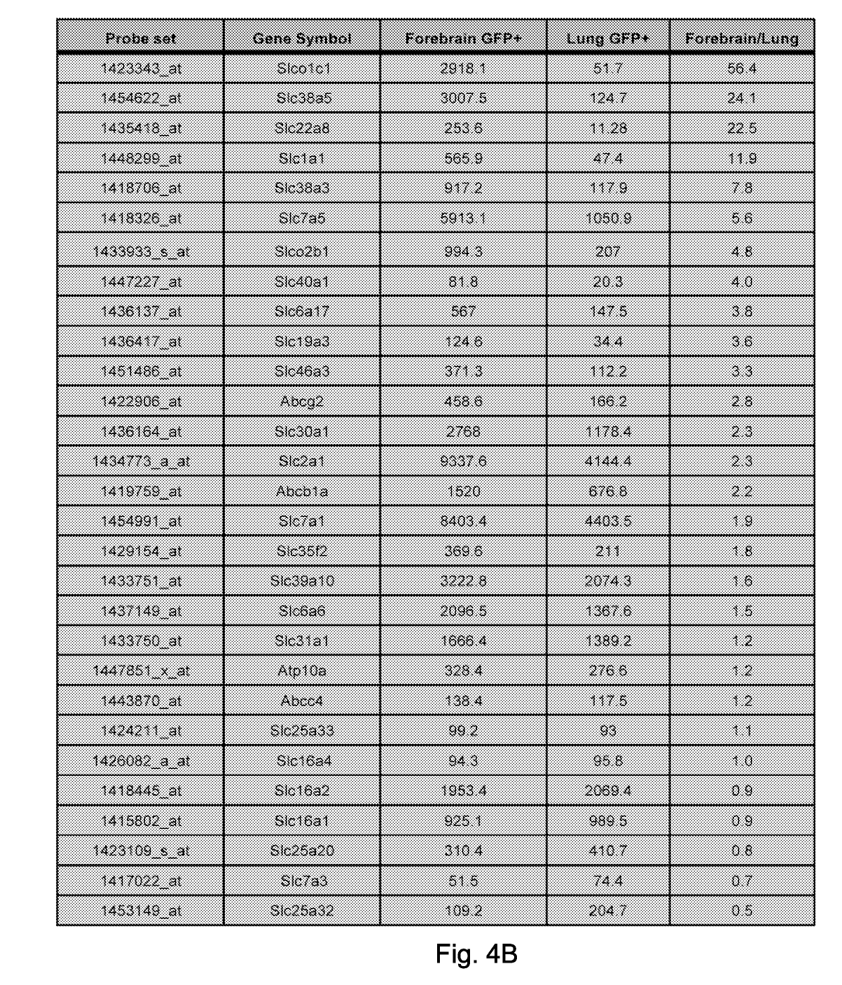

Knowing the exact timing of barrier-genesis, key molecular regulators that control the initial establishment of BBB functionality were identified in an unbiased manner by comparing expression profiles of BBB (forebrain) with non-BBB (lung) endothelium. The E13.5 forebrain was focused on at the time when the BBB is actively forming. Endothelial cells were isolated from forebrain or lung of E13.5 vascular-specific Tie2-GFP mouse embryos by fluorescence-activated cell sorting (FACS). RNA was extracted from these cell populations and an Affymetrix microarray was performed. As expected from a comparison of two endothelial populations, 91% of the genes analyzed showed less than a 2-fold difference in the relative representation of their transcripts (FIG. 1A between the lines). In both populations, many endothelial specific genes show a high representation of their transcripts while neuronal, astrocyte or pericyte specific transcripts have a negligible to very low representation, indicating a high enrichment of endothelial cells in the isolation procedure (FIG. 1B and table at FIG. 4A). 659 genes that show more than a 5-fold higher representation of their transcripts in the forebrain than in the lung endothelium were identified (FIG. 1A). Among them, many genes involved in transport are already highly and differentially expressed in brain endothelial cells (e.g. Glut1 FIG. 1C and table at FIG. 4B). As described herein, these proteins can control and/or regulate BBB differentiation. Some of these transport genes are found to be expressed in the CNS blood vessels as early as E9.5 when the peri-neural vascular plexus (PNVP) vessels just begin to ingress into the brain.

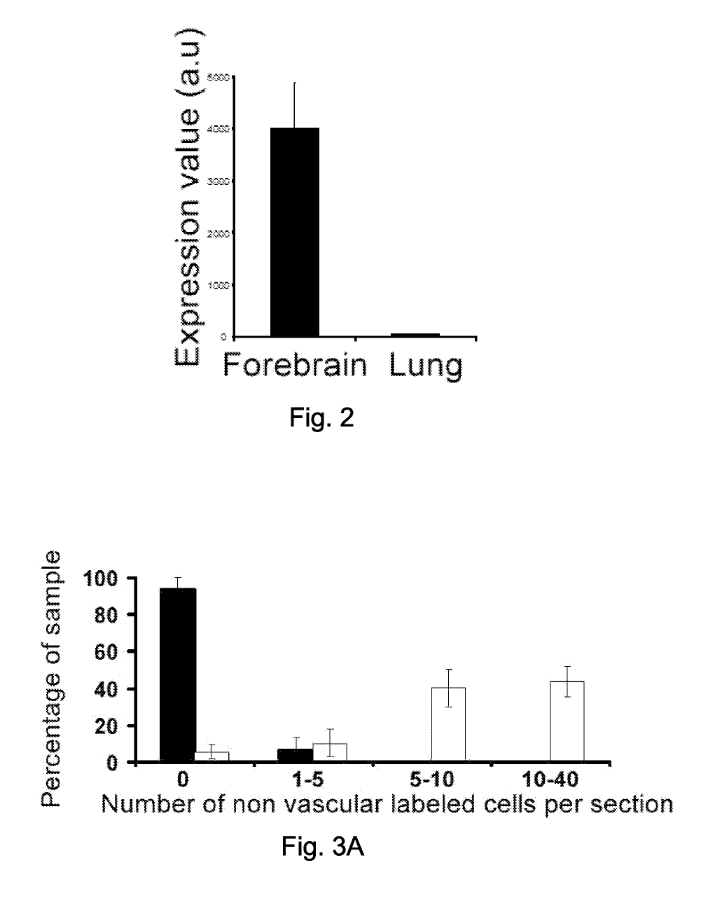

Next, candidates from the expression profile analysis that exhibited high differential expression were validated by examining their expression in the developing mouse. One of the genes, major facilitator super family domain containing 2a (Mfsd2a), showed 78.8 times higher expression in forebrain endothelium compared to lung endothelium. (FIG. 2). Strikingly, in situ hybridization analysis showed prominent Mfsd2a mRNA expression in the CNS vasculature with no detectable signal in the vasculature outside of CNS (data not shown). Moreover, Mfsd2a mRNA is not expressed in the vasculature of the circumventricular organs, which are part of the CNS, but their vasculature does not posses BBB characteristics. BBB-specific expression of Mfsd2a was observed both at embryonic (E13.5, E15.5) and postnatal stages (postnatal day (P) 2) (data not shown).

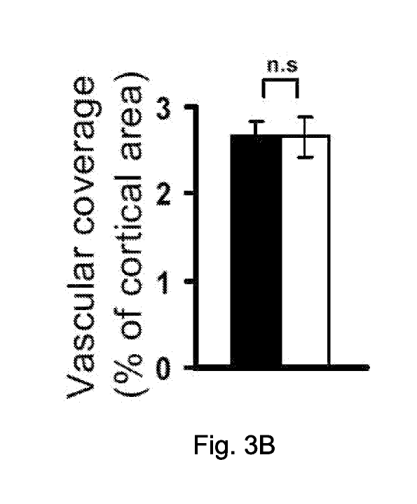

To address the requirement of MFSD2A in the establishment of functional BBB in vivo, the integrity of the BBB in mice lacking MFSD2A was examined. Using the embryonic injection method, 10 kDa dextran was injected into Mfsd2a^{-/-} and wild type littermates at E15.5-E16.5. As predicted, dextran is completely confined within the vessels in the control littermates embryos. In contrast, apparent dextran leakage to the outside of the vessels was observed in the brain of Mfsd2a^{-/-} embryos. A significant amount of diffuse patterns of dextran were found in the forebrain parenchyma, as well as individual parenchyma cells that uptake the dextran. Quantification of this phenotype was done in the developing lateral cortical plate by counting tracer positive parenchyma cells per cortical plate area (FIG. 3A). Moreover, the leaky phenotype persisted in newborn and early postnatal mice. 10 kDa Ruby-Dextran tracer injections of Mfsd2a^{-/-}/wild-type litter mates at P2-P4 revealed aberrant barrier function in the absence of Mfsd2a, Confocal microscope images revealed brain parenchyma cells stained with tracer in Mfsd2a^{-/-} but not in controls. At least 6 embryos of 3 different litters in each genotype were used. Confocal images were taken from tissue cryosections of P4 cortex (data not shown). These data demonstrate that Mfsd2a is required for the establishment of a functional barrier in vivo. To rule out the possibility that the leaky phenotype is due to abnormal blood vessel formation (or abnormal angiogenesis) in the brain, the vascular patterning in mfsd2a mutant mice was examined. In contrast to a severe barrier leakage defect, no detectable patterning difference between mfsd2a ko brain and their littermate controls was found (FIG. 3B). Therefore, MSFD2A is critical for proper embryonic barrier-genesis but not for angiogenesis in vivo.

There was no apparent difference in expression or localization of BBB markers between Mfsd2a^{-/-} and Mfsd2a^{+/+} littermates as tested by immunohistochemistry (co-staining of vessels with lectin and antibodies against the indicated markers). BBB identity of forebrain vasculature is not changed in Mfsd2a^{-/-} mice. Both mutants and control cortical plate vessels have similar signal of Glut1. Non BBB vessels at the choroid plexus of both mutants and control express PLVAP (a marker of non BBB vessels) while adjacent cortical vessels are negative. Tight-junction proteins expression is not changed in Mfsd2a^{-/-} mice. No apparent difference between Mfsd2a^{-/-} and Mfsd2a^{+/+} with regard to expression or localization of three tight-junction molecules; claudin5, ZO-1 and occluding. At least 6 embryos of 3 different litters in each genotype were used. All confocal images were taken from tissue cryosections of E15.5 cortical plates in dorsal forebrain (data not shown).

Described herein is the development of a novel and sensitive method to detect the integrity of the BBB functionality during embryonic development. Using this method, a clear temporal and spatial development profile of BBB functionality was observed and it was demonstrated that as early as E15.5, the BBB is already functional. This finding clarifies the debate in the BBB field on whether barrier-genesis occurs during embryonic development or only after birth′ and provides an important time window for studying BBB formation. This method has been applied herein to both identifying and testing barrier-genesis molecular candidates.

The recent renaissance in embryonic BBB research has yielded a series of studies relating several molecular pathways to the development of the embryonic BBB (e.g. wnt/βcatenin, Sonic Hedgehog, DR6/TROY death receptors, Retinoic acid, GPR124 and Norrin)⁶⁻¹³ Most of these studies have shown changes in the expression of BBB markers (e.g. Glut1, Claudin5 and PLVAP) upon disruption of a single gene or a molecular pathway. Nevertheless, expression changes of markers do not necessarily indicate a non-functional BBB. With no conclusive evidence for the developmental time point at which the barrier becomes functional, it is hard to clearly state whether a molecular pathway is important for barrier-genesis.

Are barrier-genesis and angiogenesis separable? Previous studies show that the disruption of wnt/βcatenin, DR6/TROY and GPR124 pathways results in BBB defects, but these mice also have severe CNS angiogenesis defects. Is the barrier phenotype secondary to the angiogenesis defects? Or perhaps these pathways influence both angiogenesis and barrier-genesis. Therefore an outstanding question in the field is whether barrier genesis and angiogenesis are coupled. It is demonstrated herein that mice lacking mfsd2a have no detectable angiogenesis defect, yet exhibit dramatic BBB leakage, demonstrating that msfd2a is specifically required for barrier-genesis but not for angiogenesis. This result, together with the finding described herein that while angiogenesis ingression in cortex occurs at E10-E11 but functional BBB is not fully formed until E15.5, further demonstrates that angiogenesis and barrier genesis are two separate events.

Mice lacking mfsd2a exhibit leaky BBB at E15.5 when the BBB just becomes functional and the leakiness continues to postnatal stages, thus mfsd2a is required for the initial establishment of a functional BBB during development. MFSD2A was reported to be expressed in placenta and testis, both organs with highly restrictive barrier properties²¹. Without wishing to be bound by theory, MFSD2A might regulate cell fusion at the BBB. In addition, MSFD2A was shown to facilitate transport of tuncamycin into cancer cell lines²³. This function is in line with its sequence similarity with major facilitator superfamily of transporters even though the physiological substance transported by MFSD2A has not been identified. Therefore, without wishing to be bound by theory, mfsd2a could also act as a carbohydrate transporter in the CNS blood vessels to modulate BBB integrity.

### REFERENCES

1. Saunders, N. R., Liddelow, S. A. & Dziegielewska, K. M. Barrier mechanisms in the developing brain, Front Pharmacol. 3, 46 (2012).
2. Daneman, R., Zhou, L., Kebede, A. A. & Barres, B. A. Pericytes are required for blood-brain barrier integrity during embryogenesis. Nature 468, 562-566 (2010).
3. Armulik, A. et al. Pericytes regulate the blood-brain barrier. Nature 468, 557-561 (2010).
4. Bell, R. D. et al. Pericytes Control Key Neurovascular Functions and Neuronal Phenotype in Adult Brain and during Brain Aging. Neuron 68, 321-323 (2010).
5. Zlokovic, B. V. The blood-brain barrier in health and chronic neurodegenerative disorders. Neuron 57, 178-201 (2008).
6. Zhong, Z. et al. ALS-causing SOD1 mutants generate vascular changes prior to motor neuron degeneration. Nature Neuroscience 11, 420-422 (2008).
7. Bell, R. D Zlokovic, B. V. Neurovascular mechanisms and blood-brain barrier disorder in Alzheimer's disease. Acta Neuropathol. 118, 103-113 (2009).
8. Bell. I. D. et al. Apolipoprotein E controls cerebrovascular integrity via cyclophilin A. Nature. 485, 512-516 (2012).
9. Reese T. S. & Karnovsky M. J. Fine structural localization of a blood-brain barrier to exogenous peroxidase. J Cell Biol. 34, 207-17 (1967).
10. Saunders, N. R. et al. Transporters of the blood-brain and blood-CSF interfaces in development and in the adult. Mol Aspects Med. 34, 742-752 (2013).
11. Stenman, J. M. et al. Canonical Writ signaling regulates organ-specific assembly and differentiation of CNS vasculature. Science 322, 1247-1250 (2008).
12. Liebner, S. et al. Wnt/beta-catenin signaling controls development of the bloodbrain barrier, J. Cell Biol. 183, 409-417 (2008).
13. Daneman, R. et al. Wnt/b-catenin signaling is required for CNS, but not non-CNS, angiogenesis. Proc. Natl Acad. Sci. USA. 106, 641-646 (2009).
14. Tam, S. J. et al. Death receptors DR6 and TROY regulate brain vascular development. Dev Cell. 22, 403-17 (2012).
15. Cullen, M. et al. GPR124, an orphan G protein-coupled receptor, is required for CNS-specific vascularization and establishment of the blood-brain barrier, Proc Natl Acad Sci USA. 108, 5759-6 (2011).
16, Wang, Y. et al. Norrin/Frizzled4 Signaling in retinal vascular development and blood brain barrier plasticity. Cell 151, 1332-44 (2012).
17. Alvarez, J. I. et al. The Hedgehog pathway promotes blood-brain barrier integrity and CNS immune quiescence. Science 334, 1727-31 (2011).
18. Mizee, M. R. et al. Retinoic acid induces blood-brain barrier development. J. Neurosci. 33, 1660-7|(2013).
19. Stern, L., Rapoport, J. L. & Lokschina, E. S. Lefonctionnement de labarrièrehémato-encéphalique chezlesnouveaunés. C. R. Soc. Biol. 100, 231-223 (1929),
20. Marin, O. & Rubenstein, J. L. A long, remarkable journey: tangential migration in the telencephalon. Nat Rev Neurosci. 2, 780-790 (2001).
21. Sheppard, A. M., Hamilton, S. K. & Pearlman, A. L. Changes in the distribution of extracellular matrix components accompany early morphogenetic events of mammalian cortical development. J. Neurosci. 11, 392@-42 (1991).
22. Esnault, C. A. placenta-specific receptor for the fusogenic, endogenous retrovirus-derived, human syncytin-2. Proc Natl Acad Sci USA. 105, 17532-72008 (2008).
23. Tang, T, et al. A mouse knockout library for secreted and transmembrane proteins. Nat Biotechnol. 28, 749-55 (2010).
24. Reiling, J. H. et al. A Haploid genetic screen identifies the major facilitator domain containing 2A (MFSD2A transporter as a key mediator in the response to tunicamycin. Proc Natl Acad Sci USA. 108, 11756-65 (2011).
25. Toufaily, C. et al. MFSD2a, the Syncytin-2 receptor, is important for trophoblast fusion. Placenta34, 85-8 (2013).
26. Berger, J. H. Charron, M. J. Silver, D. L. Major facilitator superfamily domain-containing protein 2a (MFSD2A) has roles in body growth, motor function, and lipid metabolism. PLoS One 7, e50629 doi: 10.1371 (2012),
27. Daneman, R. et al. The mouse blood-brain barrier transcriptome: a new resource for understanding the development and function of brain endothelial cells. PLoS One 5, e13741. doi: 10.1371 (2010).

### Example 2

The proper formation and function of the blood brain barrier (BBB) is critical for normal brain function. Understanding the molecular mechanisms governing BBB formation and function are critical for properly treating neurological disorders and psychiatric illnesses. However, how the BBB forms and functions is still a mystery. Described herein are three major findings that together have immediate and far-reaching implications for both our understanding of BBB formation and our ability to manipulate and/or restore the BBB for therapeutic purposes.

Described herein is a method to evaluate BBB functionality and the use of this method to identify the kinetics of BBB formation during brain development. It was thought previously that the BBB only becomes functional after birth, but demonstrated herein for the first time to have a clear temporal and spatial profile of BBB development and that the BBB is already functional in mice as early as E15.5. The discovery of the exact time window for barrier-genesis is a critical first step for studying the mechanisms governing BBB formation and function.

Described herein is genetic evidence demonstrating that BBB genesis is a unique biological process that is distinct from CNS angiogenesis, a result that refutes the previous view that BBB genesis and CNS angiogenesis are coupled. This close coupling may have been a logical conclusion based on all previously identified molecular pathways implicated in BBB formation, which result in both BBB defects and severe CNS angiogenesis defects when genetically disrupted. However, a gene, MSFD2A, whose genetic ablation disrupts only the BBB and not CNS angiogenesis is identified herein; this result demonstrates that the two processes are distinct and that the previous findings were likely a secondary consequence of CNS angiogenesis defects. The finding is the basis for development of BBB specific therapeutics that can selectively modulate the BBB without affecting angiogenesis.

Demonstrated herein via mouse genetics and electron microscopy is that MFSD2A is specifically required for the suppression of transcytosis in the CNS endothelial cells to maintain BBB integrity. It is well known that the barrier function of brain endothelial cells occurs through an increase in paracellular mechanisms (intercellular tight junctions) and a decrease in transcytotic mechanisms (macropinocytosis and fenestrae). The relative roles of these two mechanisms in BBB function have been, to date, uncharacterized, although most attention has been paid to sealing off potential leaks in the BBB via the formation of intercellular tight junctions. This demonstration that MFSD2A functions specifically in maintaining a low level of transcytosis but not in tightening the junction not only provides the first molecular evidence of how BBB function is regulated to maintain its integrity, but also highlights the importance of transcytosis mechanism in the overall function of the BBB. Finally, it is also demonstrated herein that endotheilal-pericyte interactions control the expression of MFSD2A, which in turn controls BBB integrity. Therefore, MFSD2A acts downstream of intercellular signaling mechanisms to act as a major regulator of BBB function.

The identification of MFSD2A as a key regulator for BBB formation and function, with Mfsd2a mutant mice exhibiting a leaky BBB but normal vascular patterning, and Mfsd2a mutant mice displaying a dramatic increased transcytosis but normal tight junctions, provides a valuable tool to address how a non-functional/leaky barrier could affect brain function and serve as a new model for understanding and addressing neurodegenerative diseases in the brain where BBB leakiness has been implicated. In addition, by virtue of it being an accessible cell surface molecule, and its specific role in regulating transcytosis, MFSD2A is itself poised to be a therapeutic target for pharmacologic BBB manipulation.

Given the importance of the BBB in normal brain function and neurodegenerative diseases, and how little is known about the molecular mechanisms of BBB formation and function, these findings fundamentally advance the field of BBB biology and permit the application of therapeutic approaches to manipulate the BBB for treating neurologic conditions.

The central nervous system (CNS) requires a tightly controlled environment free of various toxins and pathogens to provide the proper chemical composition for synaptic transmission. This environment is maintained by the ‘blood brain barrier’ (BBB), which is composed of highly specialized blood vessels whose endothelial cells display specialized tight junctions and unusually low rates of transcellular vesicular transport (transcytosis)^{1,2}. In concert with pericytes and astrocytes, this unique brain endothelial physiological barrier seals the CNS and controls substance influx and efflux³⁻⁵. While BBB breakdown has recently been associated to initiation and perpetuation of various neurological disorders, an intact BBB is a major obstacle for drug delivery to the CNS⁶⁻⁹. A limited understanding of the molecular mechanisms that control BBB formation has hampered our ability to manipulate the BBB in disease.

Described herein is the identification of mechanisms governing the establishment of a functional BBB. First, using a novel embryonic tracer injection method, temporal and spatial profiles of BBB functionality are described, and it is demonstrated that the mouse BBB becomes functional as early as embryonic day 15.5 (E15.5).

A screen for BBB-specific genes expressed during BBB formation is performed, and major facilitator super family domain containing 2a (Mfsd2a) is found to be selectively expressed in BBB-containing blood vessels in the CNS. Genetic ablation of Mfsd2a results in a leaky BBB both at E15.5 and postnatal stages, while maintaining the normal patterning of the vascular networks. Examination by electron microscopy reveals a dramatic increase in the vesicular activity in CNS endothelial cells in Mfsd2a^{-/-} mice, in absence of obvious tight junction defect. These findings demonstrate that BBB formation can be genetically dissociated from CNS angiogenesis, and identifies MFSD2A as a key regulator of BBB function that acts by specifically suppressing vesicle transcytosis in CNS endothelial cells. This study provides new insights into the temporal dynamics and mechanisms governing the formation and function BBB.

Two unique features of CNS endothelium determine the BBB integrity (FIG. 10)². One is the specialized tight junctions between a single layer of endothelial cells lining the CNS capillaries that form the physical seal between the blood and brain parenchyma², which is much “tighter” than the junctions between peripheral endothelial cells. In addition, CNS endothelial cells are characterized by unusually low rates of transcytosis, unlike peripheral endothelial cells which display active vesicle trafficking as a mean to deliver nutrients to the peripheral tissues, CNS endothelial cells express specific transporters to traffic specific nutrients across the BBB^{1,10}. However, the molecular mechanisms that give rise to these unique CNS endothelial cell-specific properties has been elusive. It is not clear when these properties are acquired during development, or whether these properties are acquired through regulation (induction or inhibition) of default properties of endothelial cells. Although recent studies have revealed the importance of several molecular pathways to the development of the embryonic BBB¹¹⁻¹⁸, disruption of these genes has effects on many aspects of blood vessel development, making it difficult to determine whether the barrier phenotype was primary or rather secondary to changes in vasculature network development.

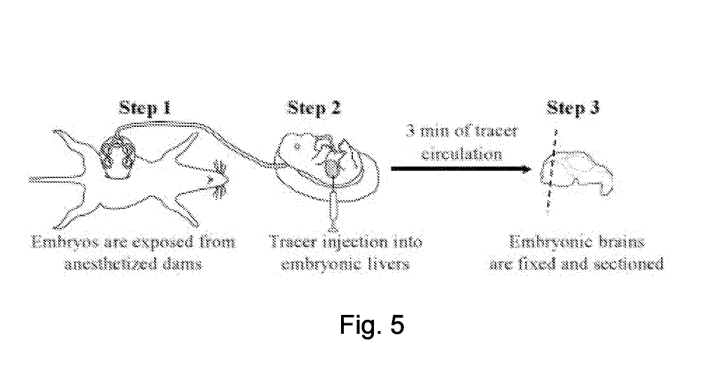

It was desired to first identify the specific time point during development when the BBB gains functional integrity. The prevailing view has been that the embryonic and even perinatal BBB is not yet functional¹. However, previous studies of embryonic and newborn BBB functionality were primarily performed by trans-cardiac dye/tracer perfusion, which may dramatically affect blood pressure, cause fragile CNS capillaries to burst, and produce an artificial leakiness phenotype^{1,19} To circumvent this obstacle, a new method was developed to assess BBB integrity during mouse development, in which a small volume of tracer is injected into embryonic liver to minimize alteration of blood pressure (See FIG. 5 and below herein for a full description of the method).

This method was used to determine the precise timing of BBB formation in the developing mouse brain and a spatial and temporal pattern of functional barrier-genesis was observed (data not shown). It was found that in the cortex at E13.5, most of the 10 kDa dextran-tracer leaked out of the brain capillaries and was subsequently taken up by non-vascular brain parenchyma cells. At E14.5, this tracer was primarily restricted to the capillaries, but diffused tracer could still be detected outside of the vessels. In contrast, at E15.5, all of the tracer was confined to vessels with no detectible signal in the surrounding brain parenchyma, similar to the mature BBB. These data demonstrate that following vessel ingression into the neural tube, the BBB gradually becomes functional as early as E15.5. In addition to this temporal profile, it was also observed a spatial pattern in BBB functionality across different regions of the brain. At E14.5, although cortical vasculature did not yet exhibit a functional barrier, midbrain and hindbrain vasculature was already capable of preventing 10 kDa dextran leakage (data not shown). Spatial differences were also apparent within the cortex (data not shown). At E13.5, most of the injected tracer escaped vessels in dorsal-medial cortex but was detected inside vessels in ventral-lateral cortex. Similarly, in the ventral-lateral cortex, the BBB was already fully sealed at E14.5 while still leaky in the dorsal-medial cortex. Therefore, BBB formation exhibits a spatial pattern in the developing cortex from ventral-lateral to dorsal-medial, similar to patterns observed in other neurodevelopmental processes such as the tangential migration path of inhibitory neuro-progenitors, deposition of extracellular matrix components, and cortical plate expansion^{20,21}

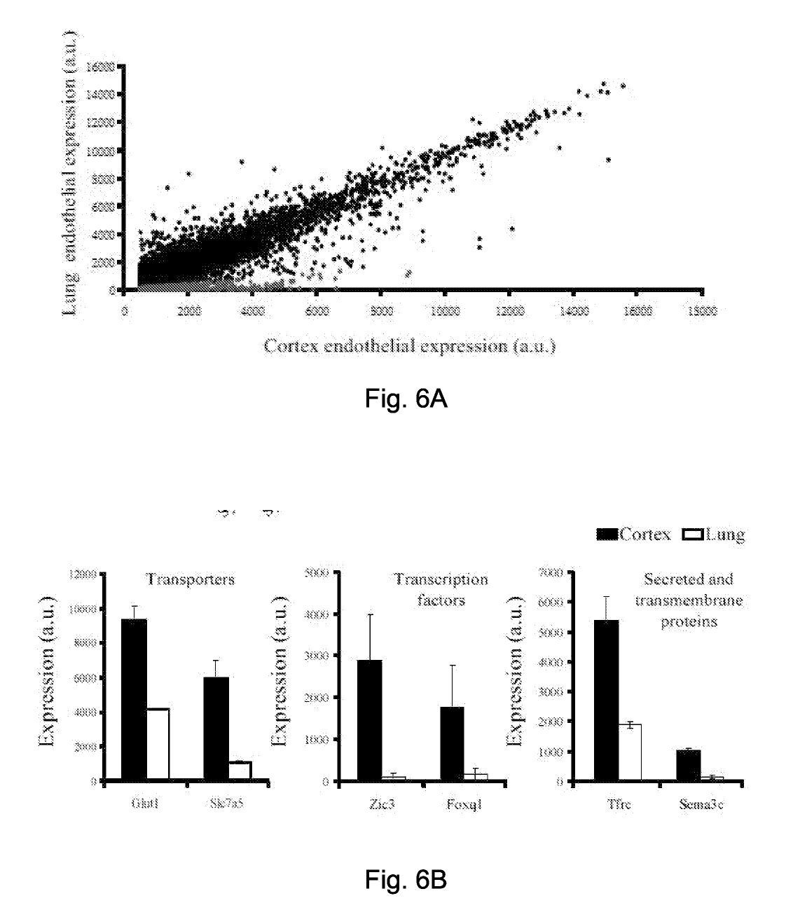

Based upon the temporal profile of BBB formation in the brain, the expression profiles of BBB (cortex) and non-BBB (lung) endothelium at E13.5 were compared, using an Affymetrix array. Cortical and lung endothelial cells were isolated from Tie2-GFP expressing mouse embryos using fluorescence-activated cell sorting (FACS). As expected from a comparison of two endothelial populations, a great majority of the genes analyzed showed little difference in the relative representation of their transcripts (FIG. 6A), with overall enrichment of endothelial-specific genes and de-enrichment of neuronal-, astrocyte- or pericyte-specific transcripts (FIGS. 1B and 4A). However, a small fraction of transcripts with significantly higher representation in the cortical endothelium than in the lung endothelium were identified. These include transporters, transcription factors, secreted and transmembrane proteins (FIGS. 6A-6B and 4B).

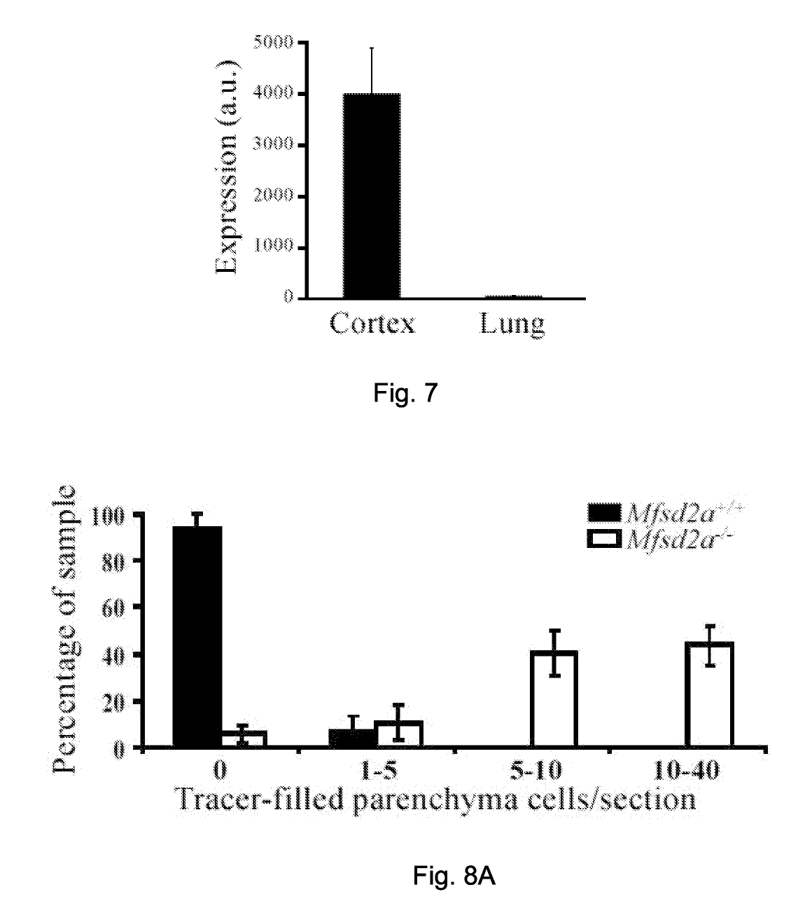

One of the genes, Mfsd2a, showed 78.8 times higher expression in cortical endothelium compared to lung endothelium in the array analysis (FIG. 7). In situ hybridization analysis showed prominent Mfsd2a mRNA expression in the CNS vasculature but no detectable signal in the vasculature outside the CNS (data not shown). Moreover, both Mfsd2a mRNA and MFSD2A protein were absent in the vasculature of the circumventricular organs or the choroid plexus, which are part of the CNS but do not posses a BBB¹ (data not shown). Mfsd2a mRNA and protein expression in CNS vasculature was observed both at embryonic (E13.5 and E15.5) and postnatal (postnatal day (P) 2 and 5) stages (data not shown). Finally, MFSD2A protein was specifically expressed in CNS endothelial cells but not in neighboring parenchymal cells (neurons and glia) nor in adjacent pericytes (data not shown). MFSD2A has also been reported to be a transmembrane protein and expressed in the placenta and testis, both organs with highly restrictive barrier properties²². Together with the demonstration of the specific expression of Mfsd2a in BBB-containing endothelial cells, this indicates that Mfsd2a may play a role in BBB formation.

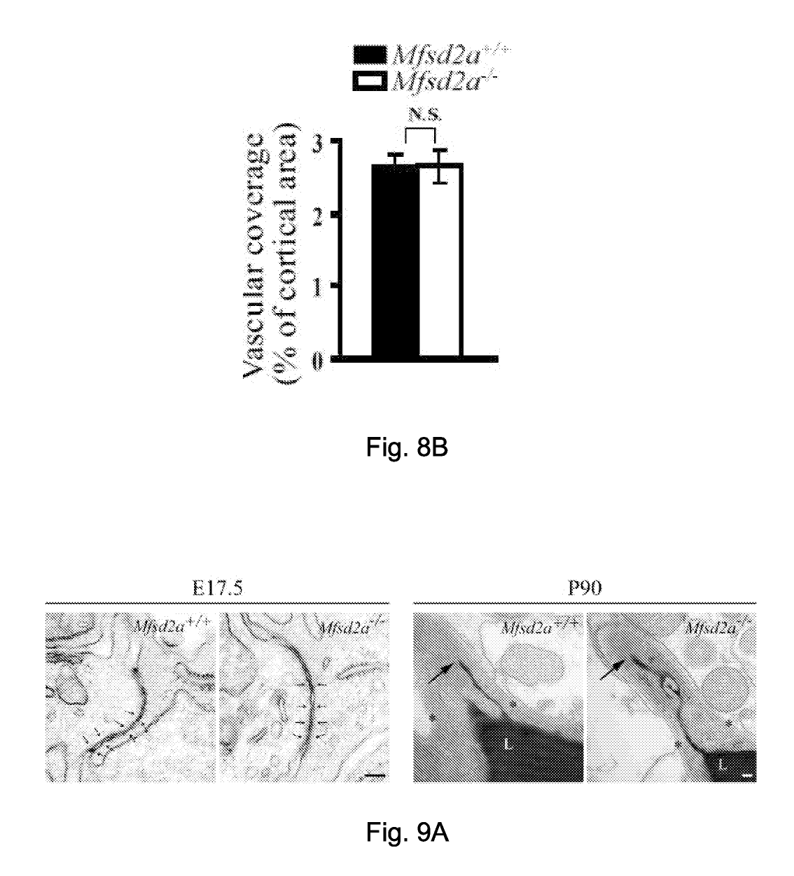

To test the possible role of MFSD2A in the establishment of a functional BBB in vivo, the integrity of the BBB was examined in mice lacking Mfsd2a²³. Using the new embryonic injection method described above herein, 10 kDa dextran was injected into Mfsd2a^{-/-} and wild type littermates at E15.5-E16.5 (data not shown). As expected, dextran is completely confined within the vessels in embryos of control littermates. In contrast, dextran leakage outside of the vessels was observed in the brain of Mfsd2a^{-/-} embryos. A substantial amount of diffused dextran was found in the cortical parenchyma, as well as individual parenchyma cells that take up the dextran. Quantification of this phenotype was done in the developing lateral cortical plate by counting tracer-positive parenchyma cells per cortical plate area (FIG. 8A). Furthermore, the leaky phenotype persisted in perinatal and early postnatal Mfsd2a^{-/-} mice (data not shown). Since MFSD2A sequence shares similarity to the major facilitator superfamily of transporters, and it has been shown to facilitate the transport of tunicamycin into cancer cell lines²⁴, it was sought to inject two non-carbohydrate-based tracers of different sizes to rule out the possibility that dextran leakiness is due to interactions with MFSD2A. All three different tracers—sulfo-NHS-biotin (˜550 Dalton), fluoro-Ruby-Dextran (10 kDa) and Horseradish peroxidase (HRP ˜44 kDa) exhibited the leaky phenotype in Mfsd2a^{-/-} mice (data not shown). Together, these data demonstrate that Mfsd2a is required for the establishment of a functional BBB in vivo.

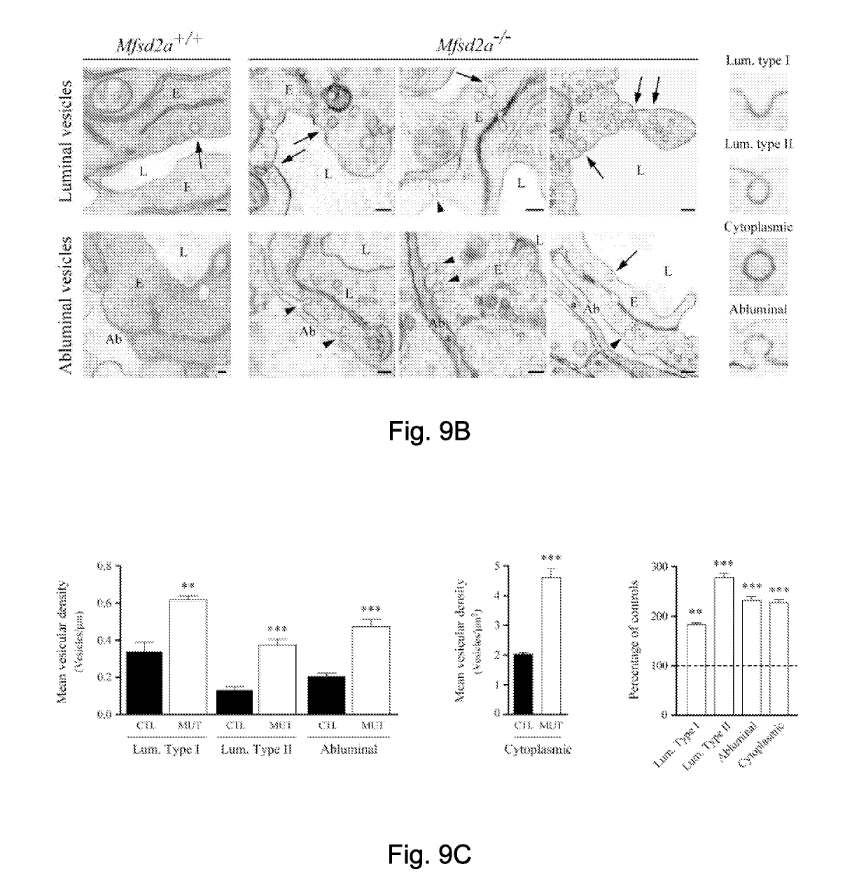

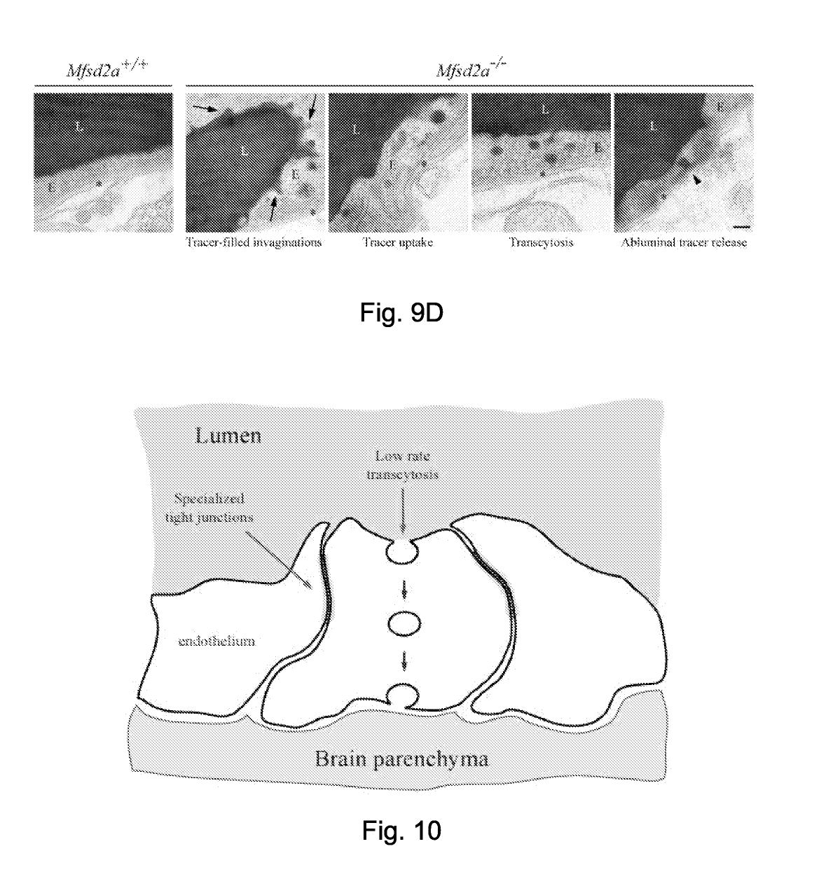

The barrier function of brain endothelial cells occurs through a reduction in the level of transcytotic mechanisms (macropinocytosis and fenestrae) relative to those observed in periphery vascular endothelial cells, and an relative increase in paracellular mechanisms (intercellular tight junctions)². The question of whether MFSD2A regulates endothelial tight junction formation, transcytosis, or both was next addressed. The integrity of these properties was examined by electron microscopy (EM) in brain samples from E17.5 embryos and P90 mice subject to intravenous Horse Radish Peroxidase (HRP) injection². EM examination failed to reveal any apparent abnormalities in the ultrastructure of endothelial tight junctions (FIG. 9A).

At E17.5, tight junctions in both control and Mfsd2a⁻/− littermates appear normal, with a typical electron-dense linear structure showing ‘kissing points’ where adjacent membranes are fused (FIG. 9A). In electron micrographs of HRP-injected adult cerebral cortex, peroxidase activity was revealed by a black (electron-dense) reaction product that filled the vessel lumen. In both control and Mfsd2a⁻/− mice, the HRP penetrated the intercellular spaces between neighboring endothelial cells only for short distance, and was stopped at the tight junction. Hence, a sharp boundary between HRP-positive and HRP-negative regions is evident without any suggestion of leakage through tight junctions. In contrast, CNS endothelial cells of Mfsd2a^{-/-} mice displayed a dramatic increase in the number of vesicles, including vesicles connected to the luminal plasma membrane, abluminal plasma membrane, and of free cytoplasmic vesicles, which may indicate an increased rate of transcytosis (FIG. 9B). Specifically, pinocytosis events were evidenced by type II lumen-connected vesicles pinching in from the luminal plasma membrane.

Quantification of the vesicles densities in different locations along the transcytosis path showed more than 2-folds increases in the vesicles number in Mfsd2a^{-/-} mice compared to control littermates (FIG. 9B). Indeed, HRP reaction product in adult mice could be observed in transcytosis vesicles invaginated from luminal membrane, and exocytosed at the abluminal plasma membrane only in Mfsd2a^{-/-} mice (FIG. 9C), suggesting that HRP itself was subject to active transcytosis in these animals. In WT littermates, HRP reaction product was confined within the vessel lumen, and the luminal plasma membrane appear non active in pinocytosis (FIG. 9C), Together, these findings indicate that the BBB leakness observed in Mfsd2a^{-/-} mice is not caused by abnormal tight junctions but rather is attributed to increased transcellular trafficking across the endothelial cytoplasm. Therefore, MFSD2A is required for the suppression of transcytosis in CNS endothelial cells to maintain BBB integrity.

A central question in the field is whether the BBB development and CNS angiogenesis are necessarily coupled or dissociated processes. Previous studies show that the disruption of wnt/βcatenin, DR6/TROY and GPR124 pathways results both in BBB defects and severe CNS angiogenesis defects in mice¹¹⁻¹⁵. Therefore, these reported barrier phenotypes could simply be a secondary effect of the angiogenesis defects. To determine whether the leaky phenotype we observed in Mfsd2a^{-/-} is accompanied by abnormal blood vessel formation, the vascular patterning in Mfsd2a^{-/-} mice was examined. In contrast to the severe barrier leakage defects (FIG. 8A), no brain vascular patterning differences were found between Mfsd2a^{-/-} and their littermate controls (FIG. 8B). Therefore, MSFD2A is specifically required for proper formation of a functional BBB but not for CNS angiogenesis in vivo. Moreover, this result, together with the temporal difference between cortical angiogenesis (E10-E11) and cortical barrier-genesis (E13.5-E15.5) demonstrates that angiogenesis and barrier-genesis are distinct processes.

In this study, a novel and sensitive method was developed to assess the integrity of the BBB during embryonic development. A clear temporal and spatial profile of BBB development was observed, and as early as E15.5, the BBB is already capable of restricting leakage of blood-borne molecules into the brain parenchyma (at least for molecular weight of 550 Da or larger). This finding provides the answer to a long-standing question, whether barrier-genesis occurs during embryonic development or only after birth¹, and identifies the time window during which the BBB forms. With no conclusive evidence for the developmental time point at which the barrier becomes functional, it has been difficult, prior to this study, to clearly state whether a molecular pathway is important for barrier-genesis.

The data presented herein also demonstrate the existence of a key regulator, MFSD2A, which is specifically expressed in BBB-containing CNS endothelial cells and is essential for the function of the BBB. Highly elaborate tight junctions and unusually low rate of transcytosis are two unique properties of CNS endothelial cells compared to the periphery (FIG. 10)². The EM investigation suggests that MFSD2A is required to suppress endothelial transcytosis activity, which is normally associated with periphery (non-BBB) vessels. In light of MFSD2A involvement in human trophoblast cell fusion²⁵ and of the genetic evidence for its role in suppressing transcytosis, MFSD2A can serve as a cell surface molecule to regulate membrane fusion or trafficking. the observation that deletion of MFSD2A increases rates of pinocytosis, taken with prior evidence that MFSD2A has been shown to facilitate the transport of tunicamycin into cancer cell lines²⁴, indicates that MFSD2A may not simply reduce the physiological rate of pinocytosis, but also act as a specific transporters of molecules such as carbohydrates

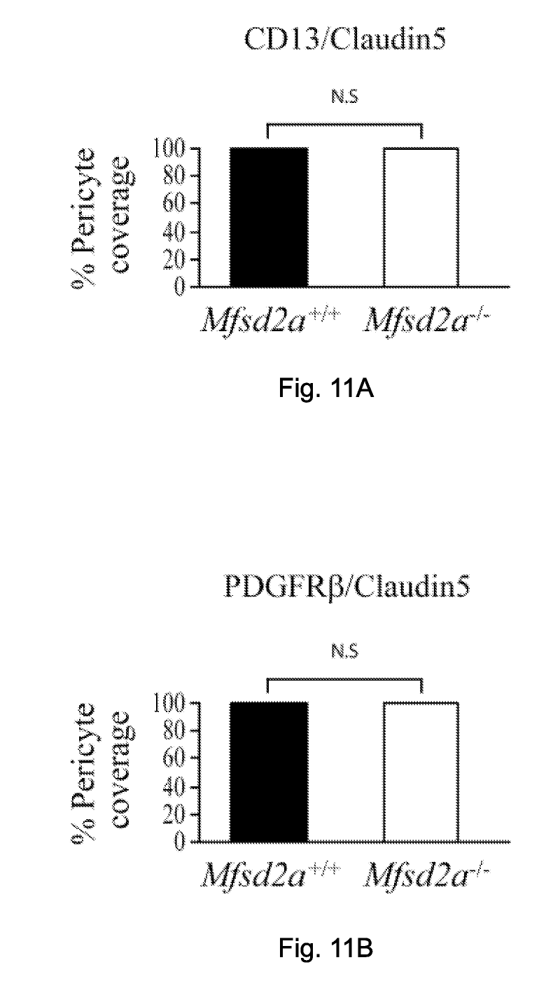

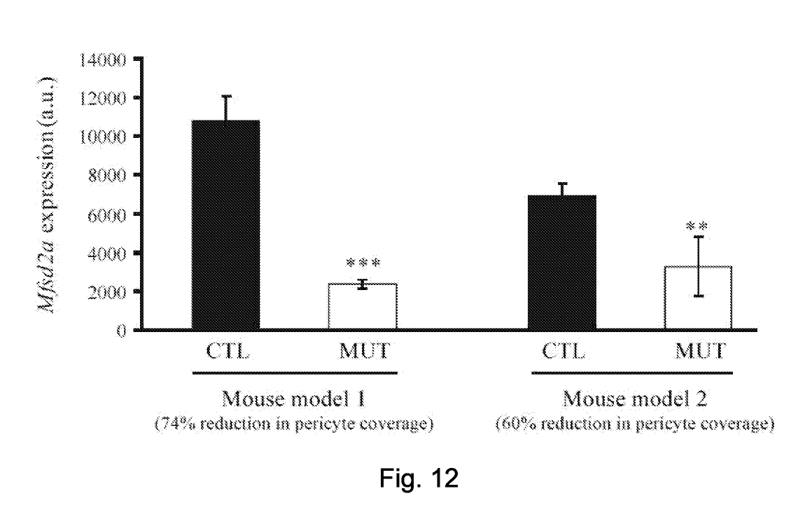

Two recent studies using genetic mouse models in which CNS vasculature has reduced coverage of pericytes have shown that pericytes can also regulate BBB integrity. Interestingly, these mouse models also displayed increased vesicle trafficking without obvious junction defect^{3,4}, similar to what we have observed in Mfsd2a^{-/-} mice. It is conceivable that the role of MFSD2A in regulating CNS endothelial transcytosis may be via modulating pericyte function, or that the effect of pericytes on endothelial transcytosis is mediated by MFSD2A. Both possibilities were examined herein. First, pericytes coverage as well as their ultrastructure and positioning relative to endothelial cells in Mfsd2a^{-/-} mice are normal (FIGS. 11A-11B). This data, together with MFSD2A-specific expression in endothelial cells but not in pericytes, suggest that the increased transcytosis observed in Mfsd2a^{-/-} mice is not due to a direct involvement of pericytes. Second, the genetically reduced pericytes coverage has been reported to influence endothelial cell gene expression profiles^{3,4}. Therefore the published microarray data of two pericyte mouse models (from Armulik, A. et al.)⁴ were analyzed and a dramatic down regulation of Mfsd2a was found in these mice, with a direct correlation between the reduction of Mfsd2a gene expression and the degree of pericyte coverage (FIG. 12)⁴. Therefore, it is possible that the vesicular phenotype observed in mice models lacking pericytes is also mediated by MFSD2A, that endotheilal-pericyte interactions control the expression of MFSD2A which in turn controls BBB integrity.

BBB breakdown has been reported in the etiology of various neurological disorders⁶⁻⁹ and two separate Mfsd2a deficient mouse lines were reported to exhibit neurological abnormalities, such as ataxic behavior^{23,26} Finding a novel physiological role of MFSD2A can provide a valuable tool to address how a non-functional BBB could affect brain development. Identifying a key molecular player in BBB formation also aids in efforts to develop therapeutic approaches to effectively penetrate the CNS. Thus, as an accessible cell surface molecule, MFSD2A is a therapeutic target for BBB restoration and manipulation

Methods

Animals.

Wild-type Swiss-Webster mice (Taconic Farms, Inc.) were used for embryonic BBB functionality assays and expression profiles. Homozygous Tie2-GFP transgenic mice (Jackson laboratory, strain 003658) were used for BBB transcriptional profiling. Mfsd2a null mice²³ (Mouse Biology Program, University of California, Davis—MMRRC strain 032467-UCD, B6; 12955-Mfsd2atm1Lex/Mmucd) were maintained on C57Bl/6; 129SVE mixed background and used for testing the involvement of MSFD2A in barrier-genesis. Pregnant mice were obtained following overnight mating (day of vaginal plug was defined as embryonic day 0.5). All animals were treated according to institutional and NIH guidelines approved by IACUC at Harvard Medical School

Immunohistochemistry.

Tissues were fixed with 4% paraformaldehyde at 4° C. overnight, cryopreserved in 30% sucrose and frozen in TissueTek OCT™ (Sakura). Tissue sections were blocked with 5% goat serum, permeabilized with 0.5% Triton X-100, and stained with the following primary antibodies: ct-PECAM (1:500; 553370, BD Pharmingen™), ct-Claudin5 (1:400; 35-2500, Invitrogen), ct-MFSD2A (1:500; Cell Signaling Technologies (under development), ct-PDGFR3 (1:100; 141402, eBioscience), ct-CD31 (1:100; 558744, BD Pharmingen™) followed by 568/488 Alexa Fluor™ conjugated secondary antibody (1:1000, Invitrogen) or with Isolectin B4 (1:500; 121411, Molecular Probes). Slides were mounted in Fluoromount™ G (EMS) and visualized by fluorescence or light microscopy.

Embryonic BBB Permeability Assay.

Deeply anaesthetized pregnant mice were used. Minimal volume (1-5 μl, 4 mg/ml) of 10 kDa Dextran-Tetramethylrhodamine, Lysine Fixable (D3312 Invitrogen) was injected into the embryonic liver while keeping the embryo connected to the maternal blood circulation through the umbilical cord. After three minutes of tracer circulation, embryonic heads were immersion fixed in 4% paraformaldehyde at 4° C. overnight, cryopreserved in 30% sucrose and frozen in TissueTek OCT (Sakura). 12 μm sections were collected and post fixed in 4% paraformaldehyde at room temperature (RT) for 15 min, washed in PBS and co-stained to visualize blood vessels with either a-PECAM antibody or with Isolectin B4 (as described above).

Transcriptional Profile.

E13.5 Tie2-GFP embryos were micro-dissected for cortex and lungs. Cortex tissue was carefully cleared of meninges and choroid plexus. FACS purification of GFP positive cells and GeneChip analysis was performed as previously described²⁷. All material from a single litter (10-13 embryos) was pooled and considered as a biological replicate. n=4 litters.

Transmission Electron Microscopy.

P90 HRP injection and E17.5 cortex capillaries TEM imaging was done as previously described². 10 mg (per 20 g) of horseradish peroxidase (Sigma Aldrich, HRP, type II) were dissolved in 0.4 ml of PBS and injected into the tail veins of deeply anaesthetized P90 mice. After 30 min HRP of circulation, brains were dissected and fixed by immersion in a 0.1 M sodium cacodylate-buffered mixture (5% glutaraldehyde and 4% formaldehyde) for 1 hr at room temperature (RT) followed by 5 hr at 4° C. Following fixation, the tissue was washed overnight in 0.1 M sodium cacodylate buffer and then cut in 50 μm thick free floating sections using a vibratome. Sections were incubated for 45 min at RT in 0.05 M Tris-HCl pH 7.6 buffer, containing 5.0 mg/10 ml of 3-3′ diaminobenzidine (DAB, Sigma Aldrich) with 0.01% hydrogen peroxide. Samples were then postfixed in 2% osmium tetroxide in sodium cacodylate buffer and treated with uranyl acetate, dehydrated and embedded in epoxy resin. E17.5 samples were processed same as the P90 samples without HRP injection and with longer fixation times (2-3 days in room temperature). Ultrathin sections (80 nm) were then cut from the block surface, collected on copper grids, stained with Reynold's lead citrate and examined under a 1200EX electron microscope (JEOL) equipped with a 2 k CCD digital camera (AMT).

### REFERENCES

1. Saunders, N. R., Liddelow, S. A. & Dziegielewska, K. M. Barrier mechanisms in the developing brain. Front Pharmacol. 3, 46 (2012).
2. Reese T. S. & Karnovsky M. J. Fine structural localization of a blood-brain barrier to exogenous peroxidase. J Cell Biol. 34, 207-17 (1967).
3. Daneman, R., Zhou, L., Kebede, A. A. & Banes, B. A. Pericytes are required for blood-brain barrier integrity during embryogenesis. Nature 468, 562-566 (2010).
4. Armulik, A. et al. Pericytes regulate the blood-brain barrier. Nature 468, 557-561 (2010).
5. Bell. R. D. et al. Pericytes Control Key Neurovascular Functions and Neuronal Phenotype in the Adult Brain and during Brain Aging. Neuron 68, 321-323 (2010).
6. Zlokovic, B. V. The blood-brain barrier in health and chronic neurodegenerative disorders. Neuron 57, 178-201 (2008).
7. Zhong, Z. et al. ALS-causing SOD1 mutants generate vascular changes prior to motor neuron degeneration. Nature Neuroscience 11, 420-422 (2008).
8. Bell, R. D., Zlokovic, B. V. Neurovascular mechanisms and blood-brain barrier disorder in Alzheimer's disease. Acta Neuropathol. 118, 103-113 (2009).
9. Bell. R. D. et al. Apolipoprotein E controls cerebrovascular integrity via cyclophilin A. Nature. 485, 512-516 (2012).
10. Saunders, N. R. et al. Transporters of the blood-brain and blood-CSF interfaces in development and in the adult. Mol Aspects Med. 34, 742-752 (2013).
11. Stenman, J. M. et al. Canonical Wnt signaling regulates organ-specific assembly and differentiation of CNS vasculature. Science 322, 1247-1250 (2008).
12. Liebner, S. et al. Wnt/beta-catenin signaling controls development of the blood-brain barrier. J. Cell Biol. 183, 409-417 (2008).
13. Daneman, R. et al. Wnt/b-catenin signaling is required for CNS, but not non-CNS, angiogenesis. Proc. Natl Acad. Sci. USA. 106, 641-646 (2009).
14. Tam, S. J. et al. Death receptors DR6 and TROY regulate brain vascular development. Dev Cell. 22, 403-17 (2012).
15. Cullen, M. et al. GPR124, an orphan G protein-coupled receptor, is required for CNS-specific vascularization and establishment of the blood-brain barrier. Proc Natl Acad Sci USA. 108, 5759-6 (2011).
16. Wang, Y. et al. Norrin/Frizzled4 Signaling in retinal vascular development and blood brain barrier plasticity. Cell 151, 1332-44 (2012).
17. Alvarez, J. I. et al. The Hedgehog pathway promotes blood-brain barrier integrity and CNS immune quiescence. Science 334, 1727-31 (2011).
18. Mizee, M. R. et al. Retinoic acid induces blood-brain barrier development. J. Neurosci. 33, 1660-71 (2013).
19. Stern, L., Rapoport, J. L. & Lokschina, E. S. Le fonctionnement de la barrière hémato-encéphalique chez les nouveau-nés. C. R. Soc. Biol. 100, 231-223 (1929).
20. Mar'n, O. & Rubenstein, J. L. A long, remarkable journey: tangential migration in the telencephalon. Nat Rev Neurosci. 2, 780-790 (2001).
21. Sheppard, A. M., Hamilton, S. K. & Pearlman, A. L. Changes in the distribution of extracellular matrix components accompany early morphogenetic events of mammalian cortical development. J. Neurosci. 11, 3928-42 (1991).
22. Esnault, C. A. placenta-specific receptor for the fusogenic, endogenous retrovirus-derived, human syncytin-2. Proc Natl Acad Sci USA. 105, 17532-72008 (2008).
23. Tang, T. et al. A mouse knockout library for secreted and transmembrane proteins. Nat Biotechnol. 28, 749-55 (2010).
24. Reiling, J. H. et al. A Haploid genetic screen identifies the major facilitator domain containing 2A (MFSD2A) transporter as a key mediator in the response to tunicamycin. Proc Natl Acad Sci USA. 108, 11756-65 (2011).
25. Toufaily, C. et al. MFSD2a, the Syncytin-2 receptor, is important for trophoblast fusion. Placenta 34, 85-8 (2013).
26. Berger, J. H. Charron, M. J. Silver, D. L. Major facilitator superfamily domain-containing protein 2a (MFSD2A) has roles in body growth, motor function, and lipid metabolism. PLoS One 7, e50629 doi: 10.1371 (2012).
27. Daneman, R. et al. The mouse blood-brain barrier transcriptome: a new resource for understanding the development and function of brain endothelial cells. PLoS One 5, e13741. doi: 10.1371 (2010).

Supplementary Information

Animals.

Mfsd2a null mice were genotyped using the following PCR primers: 5′ CCTGGTTTGCTAAGTGCTAGC (SEQ ID NO: 4) and 5′ GTTCACTGGCTTGGAGGATGC (SEQ ID NO: 5)—which provide a 210 bp product for the Mfsd2a wild-type allele. 5′ CACTTCCTAAAGCCTTACTTC (SEQ ID NO: 6) and 5′ GCAGCGCATCGCCTTCTATC (SEQ ID NO: 7)—which provide a 301 bp product for the Mfsd2a knockout allele.

Embryonic BBB Permeability Assay.

The method is based on the well-established adult BBB dye injection assay with special considerations for the injection site and volume to cater the nature of embryonic vasculature¹⁻⁴. Four major modifications were made:
1. Embryos are injected while attached via the umbilical cord to the mother's blood circulation, minimizing abrupt changes in blood flow.
2. Taking advantage of the sinusoidal/fenestrated and most permeable liver vasculature, dye is injected using a Hamilton syringe into the embryonic liver and is taken into the circulation in a matter of seconds.
3. Dye volume is adjusted to a minimum that still allows detection in all CNS capillaries after 3 minutes of circulation (high fluoresce intensity dye enables the use of small volumes and facilitates detection at the single capillary level). 1 μl for E13.5, 2 μl for E14.5, 5 μl for E15.5-E16.5
4. Traditional perfusion fixation was omitted, again to prevent damage to capillaries. Instead fixable dyes were used to allow reliable immobilization of the dye at the end of the circulation time (relatively small embryonic brain facilitates immersion fixation).
All embryos from each litter were injected blindly prior genotyping.

Postnatal BBB Permeability Assay.

P2-P4 pups were deeply anaesthetized and three methods were used:
1. 10 tl of 10 kDa Dextran-Tetramethylrhodamine (4 mg/ml D3312 Invitrogen) were injected into the left ventricle with a Hamilton syringe. After 5 min of circulation, brains were dissected and immersion fixed in 4% paraformaldehyde at 4° C. overnight, cryopreserved in 30% sucrose and frozen in TissueTek OCT (Sakura). 12 tm sections were collected and post fixed in 4% paraformaldehyde at RT for 15 min, washed in PBS and co-stained to visualize blood vessels with either α-PECAM primary antibody (1:500; 553370, BD Pharmingen™), followed by 488-Alexa Fluor conjugated secondary antibody (1:1000, Invitrogen) or with Isolectin B4 (1:500; 121411, Molecular Probes).
2. 10 tl of HRP Type II (5 mg/ml P8250-50KU Sigma-Aldrich) were injected into the left ventricle with a Hamilton syringe. After 5 min of circulation brains were dissected and immersion fixed in 2% glutaraldehyde/4% paraformaldehyde in cacodylate buffer (0.1 M, pH 7.3) at RT for 1 hour then at 4° C. for 3 hours then washed in cacodylate buffer overnight. 100 tm cortical vibratome sections were processed in a standard DAB reaction.
3. EZ-link sulfo NHS Biotin was used as a tracer as described before⁵.

Imaging.

Nikon Eclipse 80i™ microscope equipped with a Nikon DS-2™ digital camera was used to image HRP tracer experiments, vasculature coverage and pericyte coverage comparisons and expression analyses. Zeiss LSM 510 META™ upright confocal microscope was used to image Dextran and NHS-Sulfo-biotin BBB permeability assays. Nikon FluoView™ FV1000 laser scanning confocal microscope and Leica SP8 laser scanning confocal microscope were used for imaging MFSD2A and pericyte marker immunohistochemistry. Images were processed using Adobe Photoshop™ and ImageJ™ (NIH).

In Situ Hybridization.

Tissue samples were frozen in liquid nitrogen and embedded in TissueTek™ OCT (Sakura). Sections (18 μm) were hybridized with a digoxigenin (DIG)-labelled mouse Mfsd2a antisense riboprobe (1,524-2,024 bp NM_029662) at 60° C. overnight. A sense probe was used to ensure signal specificity. For detection, signals were developed using anti-DIG antibody conjugated with alkaline phosphatase (Roche). After antibody treatment, sections were incubated with BM Purple™ AP Substrate (Roche).

Quantification of Cortical Vessel Coverage.

Epi-fluorescence microscopy images of PECAM-vascular staining were analyzed with an ImageJ™ (NIH) macro. PECAM positive profiles were masked and accumulative area was calculated as percentage of total cortical plate area (manually marked according to nuclei stained with DAPI). 12 μm coronal sections of the same rostral-caudal position were used for the analysis. The same acquisition parameters were applied to all images and same threshold was used for producing masks for vascular profiles. Quantification was done blindly.

Quantification of Cortical Vessel Pericyte Coverage.

Pericyte coverage quantification was done as previously described⁶.

Quantification of Vessel Leakage.

Epi-fluorescence microscopy images of injected tracer co-stained with lectin for vascular labeling were manually analyzed with ImageJ™ (NIH). 12 μm coronal cortical sections of the same rostral-caudal position were used for the analysis. The same acquisition parameters were applied to all images and same threshold was used. Tracer positive cells found outside a vessel (parenchyma) were used as a parameter for leakage. For each embryo at least 20 sections of lateral cortical plate were scored using the same cortical plate area. Four arbitrary leakage groups were classified based on the number of tracer parenchyma positive cells per section (0, 1-5, 5-10, and 1040). Average representation of each leakage group was calculated for Mfsd2a^{-/-} and controls embryos. Quantification was done blindly.

Statistical Analysis.

Comparison between wild-type and Mfsd2a^{-/-} vascular or pericyte coverage was performed by a Mann-Whitney U test (appropriate for small sample size—each embryo was considered as a sample) using StatXact (Cytel Software Corporation, Cambridge, Mass., USA).

Transcriptional Profile.

RNA was purified with Arcturus PicoPure RNA™ isolation kit (Applied Biosystems™), followed by NuGEN™ Ovation V2 standard linear amplification and hybridization to Affymetrix Mouse Genome 430 2.0 Array™. Four biological replicates were used. Each biological replicate represents purification from different litters performed on different days, where material from 10-13 embryos in each litter was pooled.

Transcriptional Profile Analysis of Pericyte Deficient Study.

Expression data from pericyte deficient mice generated by Armulik et al. were obtained from the Gene Expression Omnibus (http://www.ncbi.nlm.nih.gov/geo, GSE15892). All microarrays were analyzed using MASS probe set condensation algorithm with Expression Console software (Affymetrix). P-value was determined using two tailed students t-test (n=4).

### SUPPLEMENTARY REFERENCES

1. Stern, L., Rapoport, J. L. & Lokschina, E. S. Le fonctionnement de la barrière hémato-encéphalique chez les nouveau-nés. C. R. Soc. Biol. 100, 231-223 (1929).
2. Ek, C. J., Habgood, M. D., Dziegielewska, K. M. & Saunders, N. R. Functional effectiveness of the blood-brain barrier to small water-soluble molecules in developing and adult opossum (Monodelphisdomestica). J. Comp. Neurol. 496, 13-26 (2006).
3. Risau, W., Hallmann, R. & Albrecht U. Differentiation-dependent expression of proteins in brain endothelium during development of the blood-brain barrier. Dev Biol. 117, 537-45 (1986).
4. Bauer, H. et al. Ontogenic expression of the erythroid-type glucose transporter (Glut1) in the telencephalon of the mouse: correlation to the tightening of the blood-brain barrier. Brain Res Dev Brain Res. 26, 317-25 (1995).
5. Wang, Y. et al. Norrin/Frizzled4 Signaling in retinal vascular development and blood brain barrier plasticity. Cell 151, 1332-44 (2012).
6. Armulik, A. et al. Pericytes regulate the blood-brain barrier. Nature 468, 557-561 (2010)

TABLE 1
Vesicular activity in the brain endothelial cells is dramatically increased in
Mfsd2a^{-/-} mice. Quantification of the vesicular density (both total and individual type of vesicles) in
E17.5 control and mutant endothelium. Mean vesicles density was calculated from the number of
vesicles types per μm of luminal membrane (luminal type I and type II vesicles), per μm² of
cytoplasm (cytoplasmic vesicles), and per μm of abluminal membrane (abluminal vesicles). Values
are mean ± S.E.M. from 4 controls and 4 mutants (10 vessels per animal, 2 images at 12,000X per
vessel). **P < 0.01, ***P < 0.001 in Student's t test.
Density of vesicles in the embryo brain endothelium (E17.5)

| | No. of | | Mean vesicular density | | | |
|---|---|---|---|---|---|---|
| Tissue | endothelial | No. of | Luminal type I | Luminal type II | Cytoplasmic | Abluminal |
| source | profiles | vesicles | vesicles (/μm) | vesicles (/μm) | vesicles (/μm²) | vesicles (/μm) |
| Controls | 40 | 1180 | 0.34 ± 0.05 | 0.14 ± 0.03 | 2.04 ± 0.06 | 0.21 ± 0.03 |
| Mfsd2a^{-/-} | 40 | 2449 | 0.62 ± 0.01** | 0.38 ± 0.03*** | 4.62 ± 0.30*** | 0.48 ± 0.04*** |

### Example 3

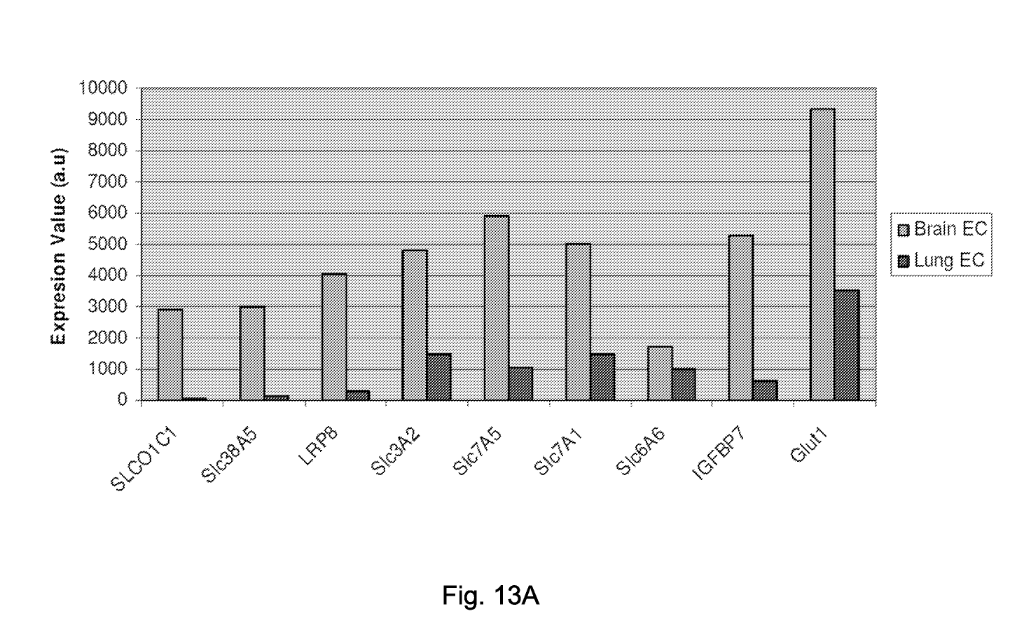

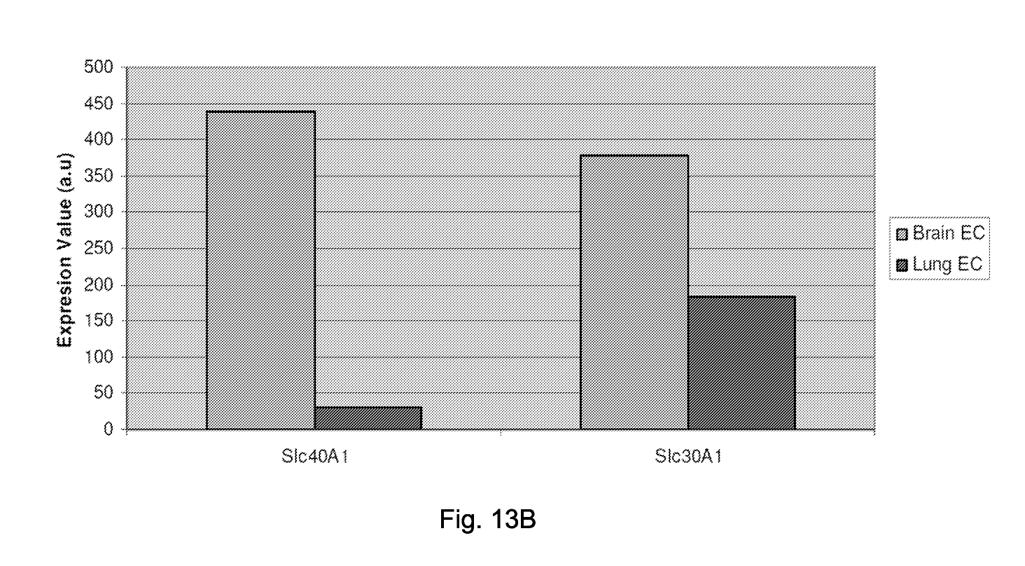

The blood-brain barrier-specific expression of Slco1C1; Slc38A5; LRP8; Slc3A2; Slc7A5; Slc7A1; Slc6A6; IGFBP7; Glut1; Slc40A1; and Slc30A1 are demonstrated in FIGS. 13A-13B.

### Example 4

### Mfsd2a is Critical for the Formation and Function of the Blood-Brain Barrier

The central nervous system (CNS) requires a tightly controlled environment free of toxins and pathogens to provide the proper chemical composition for neural function. This environment is maintained by the ‘blood-brain barrier’ (BBB), which is composed of blood vessels whose endothelial cells display specialized tight junctions and extremely low rates of transcellular vesicular transport transcytosis 1-3. In concert with pericytes and astrocytes, this unique brain endothelial physiological barrier seals the CNS and controls substance influx and efflux⁴⁻⁶. Although BBB breakdown has recently been associated with initiation and perpetuation of various neurological disorders, an intact BBB is a major obstacle for drug delivery to the CNS⁷⁻¹⁰. A limited understanding of the molecular mechanisms that control BBB formation has hindered the ability to manipulate the BBB in disease and therapy. Described herein are mechanisms governing the establishment of a functional BBB. First, using a novel tracer-injection method for embryos, spatiotemporal developmental profiles of BBB functionality are demonstrated and it is found that the mouse BBB becomes functional at embryonic day 15.5 (E15.5). Subsequently, a screen for BBB-specific genes expressed during BBB formation demonstrates that major facilitator super family domain containing 2a is selectively expressed in BBB-containing blood vessels in the CNS. Genetic ablation of Mfsd2a results in a leaky BBB from embryonic stages through to adulthood, but the normal patterning of vascular networks is maintained. Electron microscopy examination reveals a dramatic increase in CNS-endothelial-cell vesicular transcytosis in Mfsd2a−/− mice, without obvious tight-junction defects. Finally it is demonstrated that Mfsd2a endothelial expression is regulated by pericytes to facilitate BBB integrity. These findings identify Mfsd2a as a key regulator of BBB function that may act by suppressing transcytosis in CNS endothelial cells. Furthermore, these findings aid in efforts to develop therapeutic approaches for CNS drug delivery.

Two unique features of the CNS endothelium determine BBB integrity (FIG. 10). One is specialized tight junctions between a single endothelial cell layer lining the CNS capillaries, which form the physical seal between the blood and brain parenchyma². In addition, CNS endothelial cells have lower rates of transcytosis than endothelial cells in other organs³. Peripheral endothelial cells display active vesicle trafficking to deliver nutrients to peripheral tissues, whereas CNS endothelial cells express transporters to selectively traffic nutrients across the BBB^{1,3,11}. However, it is not clear when and how these properties are acquired. Furthermore, the molecular mechanisms that give rise to the unique properties of the CNS endothelium have not been identified. Although recent studies revealed molecular pathways involved in the development of the embryonic BBB¹²⁻¹⁹, disruption of some of these genes affect vascular network development, making it difficult to determine whether barrier defects are primary or secondary to a broader vascular effect.

The aim herein was to first identify the developmental time-point when the BBB gains functional integrity, and then use that time window to profile BBB-specific genes when the BBB is actively forming, to maximize the chance of identifying key regulators. The prevailing view has been that the embryonic and perinatal BBB are not yet functional¹. However, previous embryonic BBB functionality studies were primarily performed by trans-cardiac tracer perfusion, which may dramatically affect blood pressure, cause bursting of CNS capillaries, and artificially produce leakiness phenotypes^{1,20}. To circumvent these obstacles, described herein is a method to assess BBB integrity during mouse development, in which a small volume of tracer is injected into embryonic liver to minimize changes in blood pressure (FIG. 5).

Using this method, the timing of BBB formation in the developing mouse brain was identified and spatial and temporal pattern of ‘functional-barrier genesis’ was observed (data not shown). It was found that in E13.5 cortex a 10-kDa dextran tracer leaked out of capillaries and was taken up by non-vascular brain parenchyma cells (data not shown). At E14.5, the tracer was primarily restricted to capillaries, but tracer was still detected outside vessels. In contrast, at E15.5, the tracer was confined to vessels with no detectable signal in the surrounding brain parenchyma, similar to the mature BBB (data not shown). The development of BBB functionality differed across brain regions (data not shown). These data demonstrate that following vessel ingression into the neural tube, the BBB gradually becomes functional as early as E15.5.

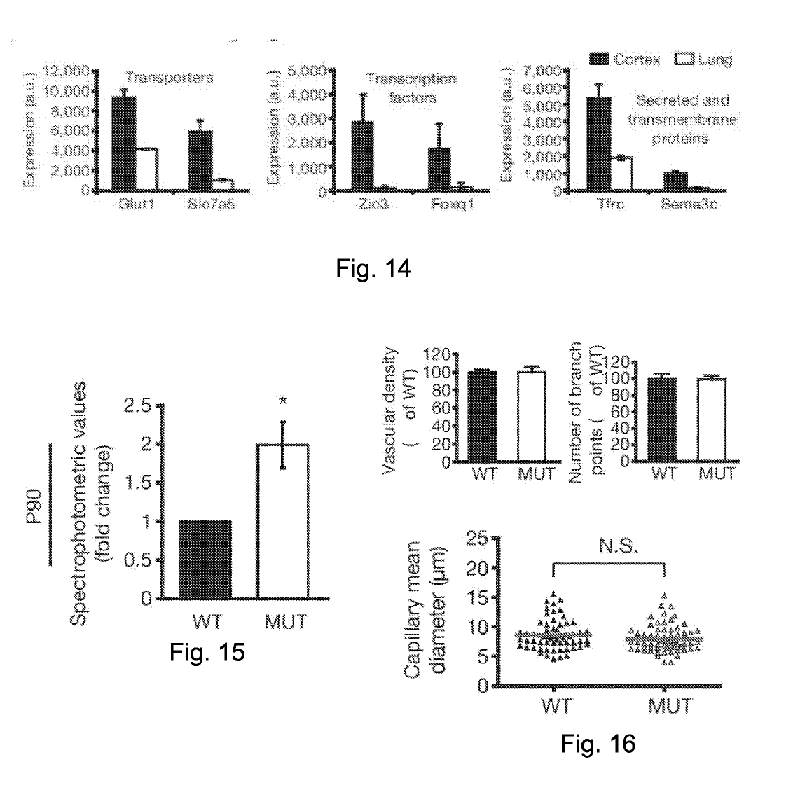

Based on the temporal profile of BBB formation, expression profiles of BBB (cortex) and non-BBB (lung) endothelium at E13.5, were compared using an Affymetrix array, and transcripts with significantly higher representation in cortical than lung endothelium were identified (FIGS. 6A-6B and 14). These transcripts included transporters, transcription factors, and secreted and transmembrane proteins (FIG. 14). There was particular interest in transmembrane proteins, owing to their potential involvement in cell-cell interactions that regulate BBB formation.

One of the genes identified, Mfsd2a, had 78.8 times higher expression in cortical endothelium than in lung endothelium (FIG. 7). In situ hybridization showed prominent Mfsd2a mRNA expression in CNS vasculature but no detectable signal in vasculature outside the CNS, such as in lung or liver (Fig. data not shown). Moreover, both Mfsd2a mRNA and Mfsd2a protein were absent in the choroid plexus vasculature, which is part of the CNS but does not possess a BBB1 (data not shown). Mfsd2a expression in CNS vasculature was observed at embryonic stages (E15.5), postnatal days 2 and 5 (P2 and P5) and in adults (P90) (data not shown). Finally, Mfsd2a protein, which is absent in the Mfsd2a−/− mice, was specifically expressed in claudin-5-positive CNS endothelial cells but not in neighbouring parenchyma cells (neurons or glia) or adjacent Pdgfrf3-positive pericytes (data not shown). Previously, Mfsd2a was reported to be a transmembrane protein expressed in the placenta and testis, which have highly restrictive barrier properties²². This demonstration of Mfsd2a-specific expression in BBB-containing endothelial cells, indicates that Mfsd2a has have a role in BBB formation and/or function.

To test this hypothesis, BBB integrity was examined in Mfsd2a−/− mice. Using the embryonic injection method described herein, 10-kDa dextran was injected into Mfsd2a−/− and wild-type littermates at E15.5. As expected, dextran was confined within vessels of control embryos. In contrast, dextran leaked outside the vessels in Mfsd2a−/− embryonic brains and was found in the cortical parenchyma (data not shown) and individual parenchyma cells (quantified as tracer-positive parenchyma cells per unit area of the developing lateral cortical plate; FIG. 8A).

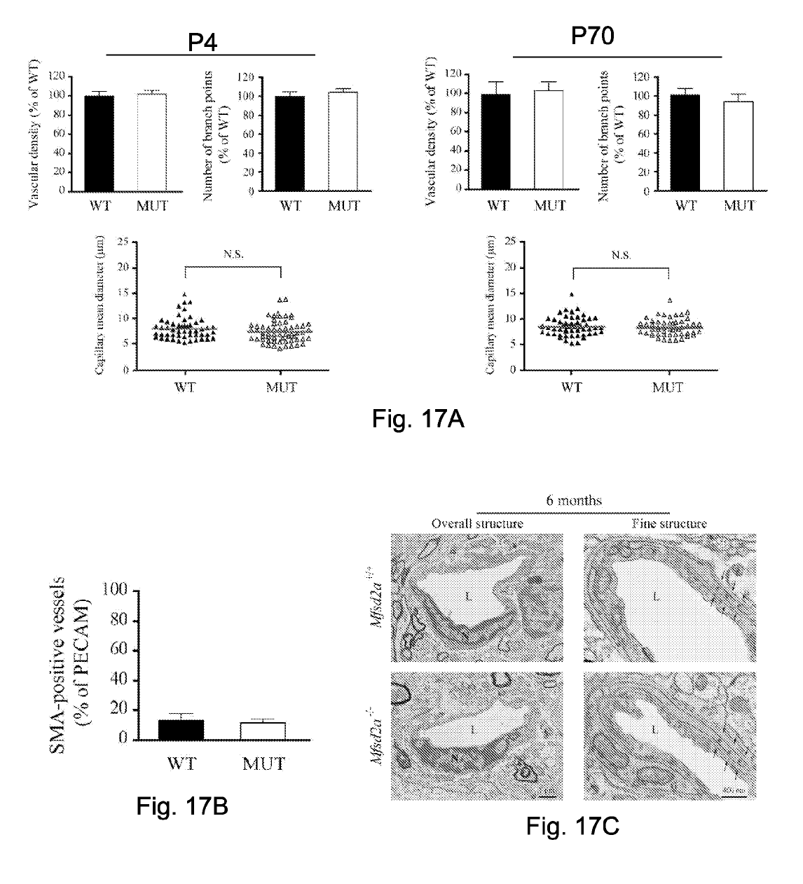

Furthermore, using imaging and spectrophotometric quantification methods⁵, it was found that the leaky phenotype persisted in early postnatal (data not shown) and adult (FIG. 15) Mfsd2a−/− mice. Because the sequence of Mfsd2a has similarities to the major facilitator superfamily of transporters, and Mfsd2a facilitates the transport of tunicamycin in cancer cell lines²³, two non-carbohydrate-based tracers of different sizes were injected to rule out the possibility that dextran leakiness is due to interactions with Mfsd2a. Sulfo-NHS-biotin (550 Da) and horseradish peroxidase (HRP; 44 kDa) tracers exhibited the leaky phenotype in Mfsd2a−/− mice (data not shown). Moreover, a larger molecular weight tracer, 70-kDa dextran, also displayed leakiness in Mfsd2a−/− mice (data not shown). In contrast to severe barrier leakage defects (FIGS. 8A and 15), brain vascular patterning was similar between Mfsd2a−/− mice and littermate controls. No abnormalities were identified in capillary density, capillary diameter or vascular branching (FIGS. 16 and 17A), in embryonic (E15.5), postnatal (P4), and adult (P70) brains of Mfsd2a−/− mice. Moreover, no abnormalities were found in cortical arterial distribution in adult Mfsd2a−/− mice (FIG. 17B). Therefore, Mfsd2a is specifically required for proper formation of a functional BBB but not for CNS vascular morphogenesis in vivo. This result, together with the temporal difference between cortical vascular ingression (E10-E11) and cortical barrier-genesis (E13.5-E15.5), demonstrates that vascular morphogenesis and barrier genesis are distinct processes.

It was next addressed whether Mfsd2a regulates endothelial tight-junction formation, transcytosis, or both. These properties were examined by electron microscopy in embryonic brains and P90 mice following intravenous HRP injection². Electron microscopy failed to reveal any apparent abnormalities in the ultrastructure of endothelial tight junctions (data not shown). At E17.5, tight junctions in control and Mfsd2a−/− littermates appeared normal, with electron-dense linear structures showing ‘kissing points’ where adjacent membranes are tightly apposed (data not shown). In electron micrographs of cerebral cortex in HRP-injected adults, peroxidase activity was revealed by an electron-dense reaction product that filled the vessel lumen. In both control and Mfsd2a−/− mice, HRP penetrated the intercellular spaces between neighbouring endothelial cells only for short distances. HRP was stopped at the tight junction, creating a boundary between HRP-positive and HRP-negative regions without leakage through tight junctions. In contrast, CNS endothelium of Mfsd2a−/− mice displayed a dramatic increase in the number of vesicles, including luminal and abluminal plasma membrane-connected vesicles and free cytoplasmic vesicles, which may indicate an increased rate of transcytosis (data not shown). Specifically, pinocytotic events were evidenced by type II lumen-connected vesicles pinching from the luminal plasma membrane. Greater than twofold increases in vesicle number in Mfsd2a−/− mice compared to control littermates were observed in different locations along the transcytotic pathway (FIG. 9C and Tables 3-4). Furthermore, the HRP reaction product in adult mice was observed in vesicles invaginated from the luminal membrane and exocytosed at the abluminal plasma membrane only in Mfsd2a−/− mice (data not shown), indicating that HRP was subject to transcytosis in these animals but not in wild-type littermates (Tables 3-4). Together, these findings indicate that the BBB leakiness observed in Mfsd2a−/− mice was not caused by opening of tight junctions, but rather by increased transcellular trafficking across the endothelial cytoplasm.

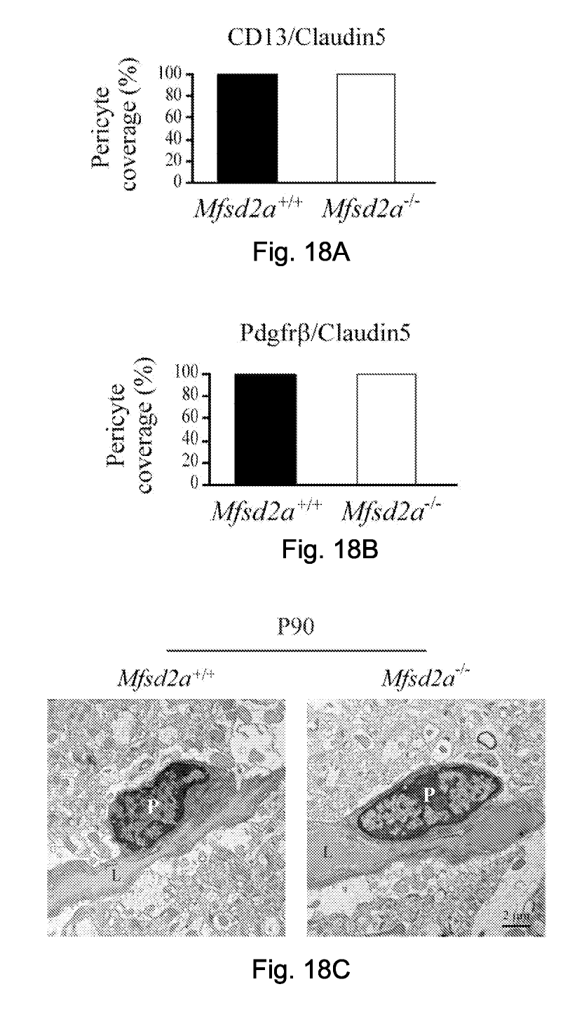

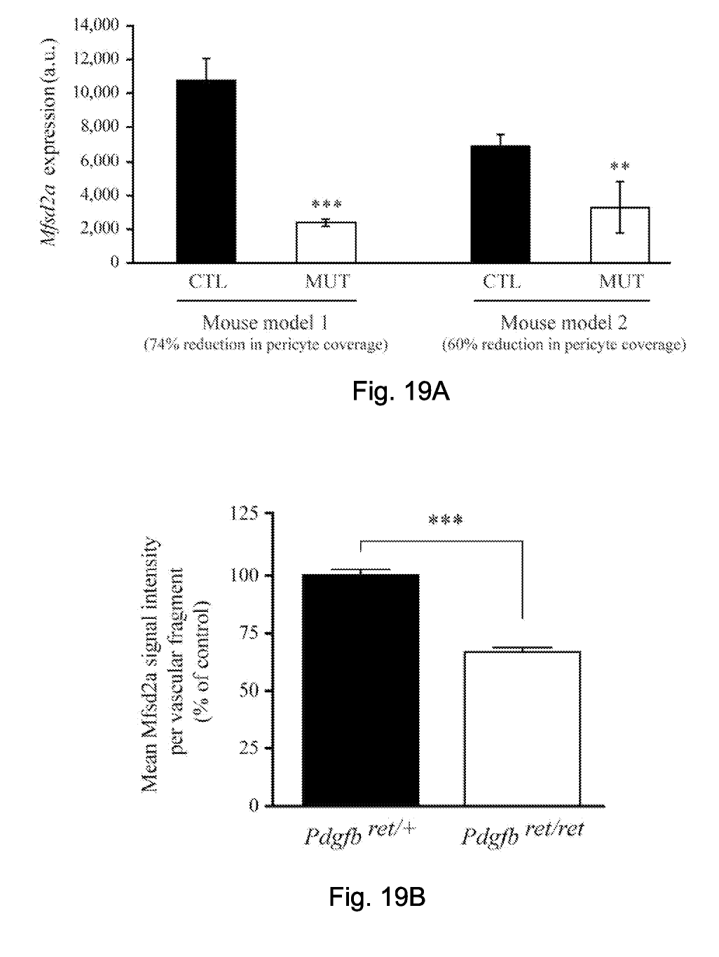

Studies using pericyte-deficient genetic mouse models have shown that pericytes can also regulate BBB integrity. These mice had increased vesicle trafficking without obvious junction defects^{4,5}, similar to the observations in Mfsd2a−/− mice. The possibilities that Mfsd2a may regulate CNS endothelial transcytosis were examined by modulating pericyte function or that the effect of pericytes on endothelial transcytosis is mediated by Mfsd2a. First, pericyte coverage, attachment to the capillary wall, and pericyte ultrastructure and positioning relative to endothelial cells were normal in Mfsd2a−/− mice (FIGS. 18A-18C). These data, together with the lack of Mfsd2a expression in pericytes, indicate that the increased transcytosis observed in Mfsd2a−/− endothelial cells is not secondary to pericyte abnormalities. Second, a genetic reduction in pericyte coverage can influence endothelial gene expression profiles. Therefore published microarray data of two pericyte-deficient mouse models were analyzed and a dramatic downregulation of Mfsd2a in these mice, with a direct correlation between the reduction of Mfsd2a gene expression and the degree of pericyte coverage was found (FIG. 19A-19B). Furthermore, immunostaining for Mfsd2a in Pdgfretaeret mice′ revealed a significant decrease in Mfsd2a protein levels in endo-thelial cells that are not covered by pericytes (FIG. 19B). Without wishing to be bound by theory, it is contemplated herein that the increased vesicular trafficking phenotype observed in pericyte-deficient mice is, at least in part, mediated by Mfsd2a, and that endothelial-pericyte interactions control the expression of Mfsd2a, which in turn controls BBB integrity.

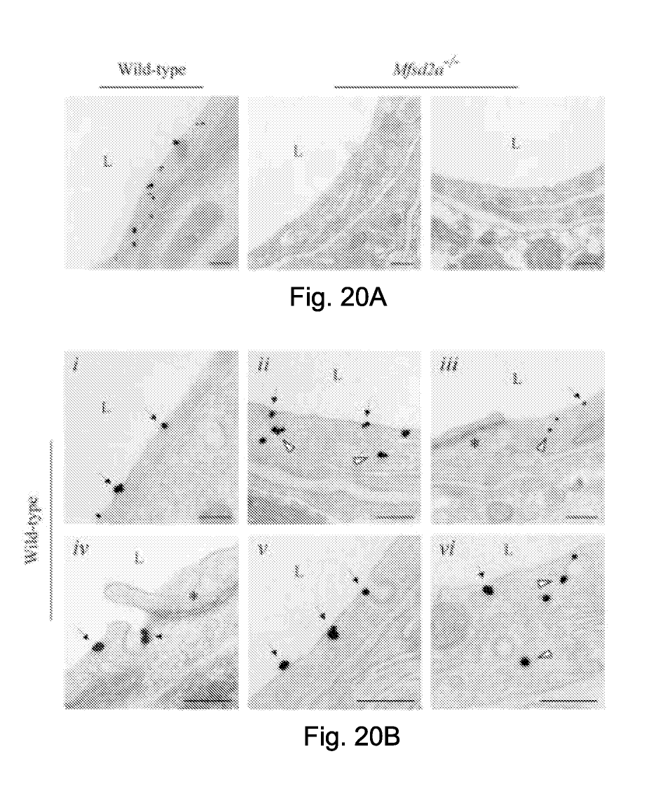

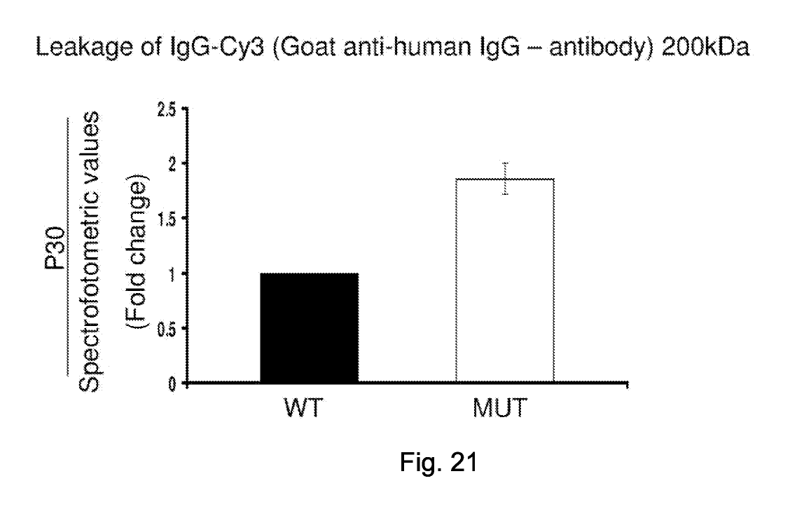

It is demonstrated herein that Mfsd2a is required to suppress endothelial transcytosis in the CNS. Because of Mfsd2a's involvement in human trophoblast cell fusion²⁴ and of our genetic evidence for its role in suppressing transcytosis, it is proposed herein that Mfsd2a serves as a cell-surface molecule to regulate membrane fusion or trafficking. Indeed, from immunoelectron-microscopy examination, Mfsd2a protein was found in the luminal plasma membrane and associated with vesicular structures in cerebral endothelial cells, but not in tight junctions (FIG. 20A-20B). At present, it is not clear whether the reported transporter function of Mfsd2a is related to its role in BBB formation.

BBB breakdown has been reported in the aetiology of various neurological disorders⁷⁻¹⁰, and two separate Mfsd2a-deficient mouse lines were reported to exhibit neurological abnormalities, such as ataxic behavior^{21,25}. Finding a novel physiological role of Mfsd2a provides a valuable tool to address how a non-functional BBB could affect brain development. In addition, the present findings also highlight the importance of the transcytotic mechanism in BBB function, whereas most previous attention has been focused on potential BBB leaks through intercellular junctions. Indeed, increased numbers of pinocytotic vesicles were observed following acute exposure to external stress inducers in animal models²⁶ and have also been observed in human pathological conditions⁹. It will be interesting to examine whether Mfsd2a is involved in these pathological and acute assault situations. Increased transcytosis in Mfsd2a-1 mice persists from embryonic stages to adulthood, and up to 6 months of age these mice exhibit no sign of vascular degeneration (FIG. 17C). The identification of a key molecular player in BBB formation may also aid efforts to develop therapeutic approaches for efficient drug delivery to the CNS. As an accessible cell surface molecule, Mfsd2a is a therapeutic target for BBB restoration and manipulation.

Methods

Animals. Wild-type Swiss-Webster mice (Taconic Farms) were used for embryonic BBB functionality assays and expression profiles. Homozygous Tie2-GFP trans-genic mice (Jackson laboratory, strain 003658) were used for BBB transcriptional profiling. Mfsd2a-null mice²¹ (Mouse Biology Program, University of California, Davis—MMRRC strain 032467-UCD, B6; 129S5-Mfsd2atm1Lex/Mmucd) were maintained on C57Bl/6; 129SVE mixed background and used for testing the involvement of Mfsd2a in barrier genesis. Mfsd2a-null mutant mice were genotyped using the following PCR primers: 5′-CCTGGTTTGCTAAGTGCTAGC-3′ (SEQ ID NO: 4) and 5′-GTTCACTGGCTTGGAGGATGC-3′ (SEQ ID NO: 5), which provide a 210-bp product for the Mfsd2a wild-type allele; and 5′-CACTTCCTAAAGCCTTACTTC-3′ (SEQ ID NO: 6) and 5′-GC AGCGCATCGCCTTCTATC-3′ (SEQ ID NO: 7), which provide a 301-bp product for the Mfsd2a-knockout allele.

Pregnant mice were obtained following overnight mating (day of vaginal plug was defined as embryonic day 0.5).

All animals were treated according to institutional and US National Institutes of Health (NIH) guidelines approved by the Institutional Animal Care and Use Committee (IACUC) at Harvard Medical School.

Immunohistochemistry. Tissues were fixed with 4 paraformaldehyde (PFA) at 4 C overnight, cryopreserved in 30 sucrose and frozen in TissueTek OCT (Sakura). Tissue sections were blocked with 5 goat serum, permeabilized with 0.5 Triton X-100, and stained with the following primary antibodies: α-PECAM (1:500; 553370, BD Pharmingen™), α-Claudin5 (1:400; 35-2500, Invitrogen), α-Mfsd2a (1:500; Cell Signaling Technologies (underdevelopment)), α-Pdgfr (1:100; 141402, eBio-science), α-CD31 (1:100; 558744, BD Pharmingen™), α-SMA (1:100; C6198, Sigma Aldrich), followed by 568/488 Alexa Fluor-conjugated secondary antibodies (1:300-1:1000, Invitrogen) or with Isolectin B4 (1:500; 121411, Molecular Probes). Slides were mounted in Fluoromount G (EMS) and visualized by epifluorescence, light, or confocal microscopy.

Hybridization. Tissue samples were frozen in liquid nitrogen and embedded in TissueTek OCT (Sakura). Sections (18 μm) were hybridized with a digoxigenin (DIG)-labelled mouse Mfsd2a antisense riboprobe (1,524-2,024 bp NM_{—} 029662) at 60 C overnight. A sense probe was used to ensure signal specificity. For detection, signals were developed using anti-DIG antibody conjugated with alkaline phosphatase (Roche). After antibody treatment, sections were incubated with BM Purple AP Substrate (Roche).

Embryonic BBB permeability assay. The method is based on the well-established adult BBB dye-injection assay with special considerations for the injection site and volume to cater the nature of embryonic vasculature^{20,28-30}.

Four major modifications were made: first, embryos were injected while still attached via the umbilical cord to the mother's blood circulation, minimizing abrupt changes in blood flow. Deeply anaesthetized pregnant mice were used. Second, taking advantage of the sinusoidal, fenestrated and most permeable liver vasculature, dye was injected using a Hamilton syringe into the embryonic liver and was taken into the circulation in a matter of seconds. Third, dye volume was adjusted to a minimum that still allows detection in all CNS capillaries after 3 min of circulation. High-fluoresce-intensity dye enables the use of small volumes and facilitates detection at the single-capillary level (10-kDa dextran-tetramethylrhodamine, lysine fixable, 4 mg ml-1 (D3312 Invitrogen), 1 μl for E13.5, 2 μl for E14.5, 5 μl for E15.5). Fourth, traditional perfusion fixation was omitted, again to prevent damage to capillaries. Instead, fixable dyes were used to allow reliable immobilization of the dye at the end of the circulation time (relatively small embryonic brain facilitates immersion fixation).

Embryonic heads were fixed by immersion in 4 PFA overnight at 4 C, cryo-preserved in 30 sucrose and frozen in TissueTek OCT (Sakura). Sections of 12 μm were then collected and post-fixed in 4 PFA at room temperature for 15 min, washed in PBS and co-stained with either α-PECAM antibody or with isolectin B4 to visualize blood vessels. All embryos from each litter were injected blind before genotyping.

Postnatal and adult BBB permeability assay. P2-P5 pups were deeply anaesthetized and three methods were used: the first method involved injection of 10 μl of 10-kDa or 70-kDa dextran tetramethylrhodamine (4 mg ml-1 D3312 Invitrogen) into the left ventricle with a Hamilton syringe. After 5 min of circulation, brains were dissected and fixed by immersion in 4 PFA at 4 C overnight, cryopreserved in 30 sucrose and frozen in TissueTek OCT (Sakura). Sections of 12 μm were collected and post-fixed in 4 PFA at room temperature for 15 min, washed in PBS and co-stained to visualize blood vessels with either α-PECAM primary antibody (1:500; 553370, BD Pharmingen), followed by 488-Alexa Fluor conjugated secondary antibody (1:1000, Invitrogen) or with isolectin B4 (1:500; I21411, Molecular Probes).

The second method involved injection of 10 μl of HRP type II (5 mg ml-1 P8250-50KU Sigma-Aldrich) into the left heart ventricle with a Hamilton syringe. After 5 min of circulation brains were dissected and immersion fixed in 2 glutaraldehyde in 4 PFA in cacodylate buffer (0.1 M, pH 7.3) at room temperature for 1 h then at 4 C for 3 h then washed in cacodylate buffer overnight. Cortical-vibratome sections (100 μm) were processed in a standard DAB reaction. The third method involved the use of EZ-link NHS-sulfo-biotin as a tracer, as described previously 17.

Imaging. Nikon Eclipse™ 80i microscope equipped with a Nikon DS-2™ digital camera was used to image HRP tracer experiments, vasculature density and pericyte coverage comparisons and expression analyses. Zeiss LSM 510 META™ upright confocal microscope was used to image Dextran and NHS-sulfo-biotin BBB permeability assays. A Nikon FluoView™ FV1000 laser scanning confocal microscope and a Leica SP8™ laser scanning confocal microscope were used for imaging Mfsd2a and pericyte marker immunohistochemistry. Images were processed using Adobe Photoshop™ and ImageJ™ (NIH).

Morphometric analysis of vasculature. Coronal sections (25-μm thick) of E15.5, P4 and P70 brains were immunostained for PECAM. For vascular density and branching, confocal images were acquired with a Nikon FluoView FV1000™ laser scanning confocal microscope and maximal projection images (5 per animal) were used for quantifications. The number of branching points was manually counted. Capillary density was quantified using MetaMorph™ software (Universal Imaging, Downingtown, Pa.) by measuring the area occupied by PECAM-positive vessels per cortical area. The mean capillary diameter was measured manually in ImageJ™ from cross-sectional vascular profiles (20 per animal) on micrographs (5-7 per animal) taken under a X60 objective with a X2 digital zoom.

For artery distribution quantification, 25-μm-thick sections (P60) were stained for smooth muscle actin (SMA) and PECAM. The proportion of PECAM-positive brain vessels with artery (SMA) identity was quantified using MetaMorph™ and expressed as percent of controls. Quantification was carried out blind.

Quantification of cortical-vessel pericyte coverage. Pericyte coverage of cortex vessels in Mfsd2a-I- and wild-type littermate control mice was quantified by analysing the proportion of total claudin-5-positive endothelial length also positive for the pericyte markers CD13 or Pdgfr. Immunostaining was performed on 20-μm sections of P5 cortex. In each animal, 20 images of 10 different sections were analysed. Microvasculature was found to be completely covered by pericytes in both control and Mfsd2a-I-mice and therefore no error bars are presented for the average pericyte coverage in FIGS. 18A-18C (n=3). All the analysis was done with ImageJ™ (NIH). Quantification was carried out blind.

Quantification of vessel leakage. Epifluorescence images of sections from injected tracer and co-stained with lectin were analysed manually with ImageJ™ (NIH). Coronal cortical sections (12 μm) of the same rostrocaudal position were used for the analysis. The same acquisition parameters were applied to all images and the same threshold was used. Tracer-positive cells found outside a vessel (parenchyma) were used as a parameter for leakage. For each embryo, at least 20 sections of a fixed lateral cortical plate area were scored. Four arbitrary leakage groups were classified based on the number of tracer parenchyma positive cells per section (0,1-5, 5-10 and 10-40). Average representation of each leakage group was calculated for Mfsd2a-I- and control embryos. Quantification was carried out blind.

Spectrophotometric quantification of 10-kDa fluoro-ruby-dextran tracer was carried out from cortical extracts, 16 h after tail-vein injections in adult mice, as described previously⁵.

Transmission electron microscopy. TEM imaging of P90 HRP injection and E17.5 cortex capillaries was carried out as described previously². HRP (10 mg (per 20 g); Sigma Aldrich, HRP type II) were dissolved in 0.4 ml of PBS and injected into the tail veins of deeply anaesthetized P90 mice. After 30 min of HRP circulation, brains were dissected and fixed by immersion in a 0.1 M sodium-cacodylate-buffered mixture (5 glutaraldehyde and 4 PFA) for 1 hat room temperature followed by 5 h in PFA at 4 C. Following fixation, the tissue was washed overnight in 0.1 M sodium-cacodylate buffer and then cut in 50-μm-thick free-floating sections using a vibrotome. Sections were incubated for 45 min at room temperature in 0.05 M Tris-HCl pH 7.6 buffer, containing 5.0 mg per 10 ml of 3-3′ diaminobenzidine (DAB, Sigma Aldrich) with 0.01 hydrogen peroxide. Sections were then post-fixed in 1 osmium tetroxide and 1.5 potassium ferrocyanide and dehydrated and embedded in epoxy resin. E17.5 samples were processed as the P90 samples without HRP injection and with longer fixation times (2-3 days in room temperature). Ultrathin sections (80 nm) were then cut from the block surface, collected on copper grids, stained with Reynold's lead citrate and examined under a 1200EX electron microscope (JEOL) equipped with a 2 k CCD digital camera (AMT) Immunogold labelling for electron microscopy. Mice were deeply anaesthetized and perfused through the heart with 30 ml of PBS followed by 150 ml of a fixative solution (0.5 glutaraldehyde in 4 PFA prepared in 0.1 mM phosphate buffer, pH 7.4), and then by 100 ml of 4 PFA in phosphate buffer. The brain was removed and post fixed in 4 PFA (30 min, 4 C) and washed in PBS.

Coronal brain sections (50-μm thick) were cut on the same day with a vibratome and processed free floating. Sections were immersed in 0.1 sodium borohydride in PBS (20 min, room temperature), rinsed in PBS and pre-incubated (2 h) in a blocking solution of PBS containing 10 normal goat serum, 0.5 gelatine and 0.01 Triton. Incubation (24 h, 20-25 C) with rabbit anti-Mfsd2a (1:100; Cell Signaling Technologies (under development)) primary antibody was followed by rinses in PBS and incubation (overnight, 20-25 C) in a dilution of gold-labelled goat anti-rabbit IgGs (1:50; 2004, Nanoprobes). After washes in PBS and sodium acetate, the size of immunogold particles was silver-enhanced and sections rinsed in phosphate buffer before processing for electron microscopy.

Statistical analysis. Comparison between wild-type and Mfsd2a-1-pericyte coverage and spectrophotometric quantification of 10-kDa fluoro-ruby-dextran tracer leakage was performed by a Mann-Whitney U-test (appropriate for small sample size; each embryo was considered as a sample). An unpaired student's t-test was used (GraphPad Prism 4™ Software) for comparison between wild-type and Mfsd2a-1- for vascular density, artery distribution, number of vesicular types, mean capillary diameter and Mfsd2a expression in pericyte deficient mice. P 0.05 was considered significant (StatXact Cytel™ Software Corporation, Cambridge, Mass., USA). Transcriptional profiling. E13.5 Tie2-GFP embryos were micro-dissected for cortex and lungs. Cortex tissue was carefully cleared of the meninges and choroid plexus. FACS purification of GFP-positive cells and GeneChip™ analysis was performed as described previously”. RNA was purified with Arcturus PicoPure™ RNA isolation kit (Applied biosystems), followed by NuGEN Ovation V2 standard linear amplification and hybridization to Affymetrix Mouse Genome 430 2.0™ Array. All material from a single litter (10-13 embryos) was pooled and considered as a biological replicate. Four biological replicates were used. Each biological replicate represents purification from different litters performed on different days.

Transcriptional profile analysis of pericyte deficient mice. Expression data from a published study of pericyte-deficient mice⁵ were obtained from the Gene Expression Omnibus (available on the world wide web at www.ncbi.nlm.nih.gov/geo, accession number GSE15892). All microarrays were analysed using the MASS probe set condensation algorithm with Expression Console™ software (Affymetrix). P values were determined using a two-tailed student's t-test (n=4).

Mfsd2a protein expression in Pdgfb^{ret/ret} mice. Sample processing and immunohistochemistry was carried out as described for all other samples in this study. Mfsd2a staining quantification was carried out with 12-μm cortical sagittal sections. Confocal images were acquired with a Nikon FluoView FV1000™ laser scanning confocal microscope. Quantification of mean grey value per vascular profile was done with ImageJ™. (NIH) by outlining vascular profiles according to lectin staining and measuring Mfsd2a intensity in these areas. In all images, Pdgfr13 antibody staining was used to test presence of pericytes in quantified vessels. n=2 animals per genotype, 60 images quantified of at least 600 vascular profiles per animal. Quantification was carried out blind.

### REFERENCES

1. Saunders, N. R., Liddelow, S. A. Dziegielewska, K. M. Barrier mechanisms in the developing brain. Front. Pharmacol. 3, 46 (2012).
2. Reese, T. S. Karnovsky, M. J. Fine structural localization of a blood-brain barrier to exogenous peroxidase. J. Cell Biol. 34, 207-217 (1967).
3. Siegenthaler, J. A., Sohet, F. Daneman, R. ‘Sealing off the CNS’: cellular and molecular regulation of blood-brain barriergenesis. Curr. Opin. Neurobiol. 23, 1057-1064 (2013).
4. Daneman, R., Zhou, Ô., Kebede, A. A. Barres, B. A. Pericytes are required for blood-brain barrier integrity during embryogenesis. Nature 468, 562-566 (2010).
5. Armulik, A. et al. Pericytes regulate the blood-brain barrier. Nature 468, 557-561 (2010).
6. Bell, R. D. et al. Pericytes control key neurovascular functions and neuronal phenotype in the adult brain and during brain aging. Neuron 68, 321-323 (2010).
7. Zlokovic, B. V. The blood-brain barrier in health and chronic neurodegenerative disorders. Neuron 57, 178-201 (2008).
8. Zhong, Z. et al. ALS-causing SOD1 mutants generate vascular changes prior to motor neuron degeneration. Nature Neurosci. 11, 420-422 (2008).
9. Bell, R. D. Zlokovic, B. V. Neurovascular mechanisms and blood-brain barrier disorder in Alzheimer's disease. Acta Neuropathol. 118, 103-113 (2009).
10. Bell, R. D. et al. Apolipoprotein E controls cerebrovascular integrity via cyclophilin A. Nature 485, 512-516 (2012).
11. Saunders, N. R. et al. Transporters of the blood-brain and blood-CSF interfaces in development and in the adult. Mol. Aspects Med. 34, 742-752 (2013).
12. Stenman, J. M. et al. Canonical Wnt signaling regulates organ-specific assembly and differentiation of CNS vasculature. Science 322, 1247-1250 (2008).
13. Liebner, S. et al. Wnt/p-catenin signaling controls development of the blood-brain barrier. J. Cell Biol. 183, 409-417 (2008).
14. Daneman, R. et al. Wnt/p-catenin signaling is required for CNS, but not non-CNS, angiogenesis. Proc. Natl Acad. Sci. USA 106, 641-646 (2009).
15. Tam, S. J. et al. Death receptors DR6 and TROY regulate brain vascular development. Dev. Cell 22, 403-417 (2012).
16. Cullen, M. et al. GPR124, an orphan G protein-coupled receptor, is required for CNS-specific vascularization and establishment of the blood-brain barrier. Proc. Natl Acad. Sci. USA 108, 5759-5764 (2011).
17. Wang, Y. et al. Norrin/Frizzled4 signaling in retinal vascular development and blood brain barrier plasticity. Cell 151, 1332-1344 (2012).
18. Alvarez, J. I. et al. The Hedgehog pathway promotes blood-brain barrier integrity and CNS immune quiescence. Science 334, 1727-1731 (2011).
19. Mizee, M. R. et al. Retinoic acid induces blood-brain barrier development. J. Neurosci. 33, 1660-1671 (2013).
20. Stern, L., Rapoport, J. L. Lokschina, E. S. Le fonctionnement de la barriere helmato-encelphalique chez les nouveau-nels. C. R. Soc. Biol 100, 231-223 (1929).
21. Tang, T. et al. A mouse knockout library for secreted and transmembrane proteins. Nature Biotechnol. 28, 749-755 (2010).
22. Esnault, C. placenta-specific receptor for the fusogenic, endogenous retrovirus-derived, human syncytin-2. Proc. Natl Acad. Sci. USA 105, 17532-17537 (2008).
23. Reiling, J. H. et al. A Haploid genetic screen identifies the major facilitator domain containing 2A (MFSD2A) transporter as a key mediator in the response to tunicamycin. Proc. Natl Acad. Sci. USA 108, 11756-11765 (2011).
24. Toufaily, C. et al. MFSD2a, the Syncytin-2 receptor, is important for trophoblast fusion. Placenta 34, 85-88 (2013).
25. Berger, J. H. Charron, M. J. Silver, D. L. Major facilitator superfamily domain-containing protein 2a (MFSD2A) has roles in body growth, motor function, and lipid metabolism. PLoS One 7, e50629 (2012).
26. Nag, S. In The Blood-Brain Barrier-Biology and Research Protocols Part 2 (ed. Nag, S.) 99-100 (2003).
27. Chen, F., Evans, A., Pham, J. Plosky, B. (eds) Mol. Cell Special Review issue 40 (2010).
28. Ek, C. J., Habgood, M. D., Dziegielewska, K. M. Saunders, N. R. Functional effectiveness of the blood-brain barrier to small water-soluble molecules in developing and adult opossum (Monodelphisdomestica). J. Comp. Neurol. 496, 13-26 (2006).
29. Risau, W., Hallmann, R. Albrecht, U. Differentiation-dependent expression of proteins in brain endothelium during development of the blood-brain barrier. Dev. Biol. 3.3.7, 537-545 (1986).
30. Bauer, H. et al. Ontogenic expression of the erythroid-type glucose transporter (Glut1) in the telencephalon of the mouse: correlation to the tightening of the blood-brain barrier. Brain Res. Dev. Brain Res. 86, 317-325 (1995).
31. Daneman, R. et al. The mouse blood-brain barrier transcriptome: a new resource for understanding the development and function of brain endothelial cells. PLoS One 5, 313741 (2010).

TABLE 3
Quantification of the vesicular density (both total and individual type of
vesicles) in E 17.5 control and mutatnt endothelium. Mean vesicular density was calculated from the
number of vesicular types per um of luminal membrane (luminal type I and type II vesicles) per um²
of cytoplasm (cytoplasmic vesicles), and per um of abluminal membrane (abluminal vesicles).
Density of vesicles in the embryo brain endothelium (E17.5)

| | No. of | | Mean vesicular density | | | |
|---|---|---|---|---|---|---|
| Tissue | endothelial | No. of | Luminal type I | Luminal type II | Cytoplasmic | Abluminal |
| source | profiles | vesicles | vesicles (/μm) | vesicles (/μm) | vesicles (/μm²) | vesicles (/μm) |
| Controls | 40 | 1180 | 0.34 ± 0.05 | 0.14 ± 0.03 | 2.04 ± 0.06 | 0.21 ± 0.03 |
| Mfsd2a^{-/-} | 40 | 2449 | 0.62 ± 0.03** | 0.38 ± 0.03*** | 4.62 ± 0.30*** | 0.48 ± 0.04*** |

TABLE 4
Quantification of HRP luminal uptake in P90 HRP-injected mice.
No HRP-filled vesicles were found in wild-type mice.
Data are mean ± s.e.m. from 4 controls and 4 mutants
(10 vessels per animal, 2 minages at ×12,000 per vessel).
Density of HRP-filled vesicles in adult brain endothelium (P90)

| | No. of | No. of | Cytoplasmic |
|---|---|---|---|
| Tisuue | endothelial | HRP-filled | HRP⁺ |
| source | profiles | vesicles | vesicles (/μm²) |
| Controls | 15 | 0 | 0 |
| Mfsd2a^{-l-} | 15 | 97 | 3.35 ± 0.55 |

1. A method of modulating the permeability of the blood-brain barrier in a subject, the method comprising:
administering an inhibitor of a gene or gene expression product selected from the group consisting of:
Mfsd2A; Slco1C1; Slc38A5; LRP8; Slc3A2; Slc7A5; Slc7A1; Slc6A6; IGFBP7; Glut1; Slc40A1; and Slc30A1
to the subject, whereby the permeability of the blood-brain barrier is increased; or
administering an agonist of a gene or gene expression product selected from the group consisting of:
Mfsd2A; Slco1C1; Slc38A5; LRP8; Slc3A2; Slc7A5; Slc7A1; Slc6A6; IGFBP7; Glut1; Slc40A1; and Slc30A1
to the subject, whereby the permeability of the blood-brain barrier is decreased.

2. A method of treatment, the method comprising
administering an inhibitor of a gene or gene expression product selected from the group consisting of:
Mfsd2A; Slco1C1; Slc38A5; LRP8; Slc3A2; Slc7A5; Slc7A1; Slc6A6; IGFBP7; Glut1; Slc40A1; and Slc30A1
to a subject in need of increased permeability of the blood-brain barrier; or
administering an agonist of a gene or gene expression product selected from the group consisting of:
Mfsd2A; Slco1C1; Slc38A5; LRP8; Slc3A2; Slc7A5; Slc7A1; Slc6A6; IGFBP7; Glut1; Slc40A1; and Slc30A1
to the subject in need of decreased permeability of the blood-brain barrier.

3. (canceled)

4. The method of claim 1, wherein the inhibitor is an inhibitor of Mfsd2A.

5. The method of claim 4, wherein the inhibitor of Mfsd2A is selected from the group consisting of:
tunicamycin; tunicamycin analogs; inhibitory anti-Mfsd2A antibodies; and inhibitory nucleic acids.

6. The method of claim 2, wherein the subject administered an inhibitor is in need of delivery of a central nervous system therapeutic agent to the central nervous system.

7. The method of claim 6, wherein the method further comprises administering a central nervous system therapeutic agent to the subject.

8. The method of claim 2, wherein the subject in need of increased permeability of the blood-brain barrier is in need of treatment for a condition selected from the group consisting of:
brain cancer; encephalitis; hydrocephalus; Parkinson's disease; neuropathic pain; and a condition treated by the administration of psychiatric drugs.

9. (canceled)

10. The method of claim 2, wherein the subject administered an agonist is in need of improved quality of tight junctions of the blood-brain barrier.

11. The method of claim 2, wherein the subject in need of decreased permeability of the blood-brain barrier is in need of treatment for a condition selected from the group consisting of:
a neurodegenerative disease; multiple sclerosis; Parkinson's disease; Huntington's disease; Pick's disease; ALS; dementia; stroke; and Alzheimer's disease.

12. A pharmaceutical composition comprising an inhibitor of a gene or gene expression product selected from the group consisting of:
Mfsd2A; Slco1C1; Slc38A5; LRP8; Slc3A2; Slc7A5; Slc7A1; Slc6A6; IGFBP7; Glut1; Slc40A1; and Slc30A1
and a pharmaceutically-acceptable carrier.

13. (canceled)

14. The composition of claim 12, wherein the inhibitor is an inhibitor of Mfsd2A.

15. The composition of claim 14, wherein the inhibitor of Mfsd2A is selected from the group consisting of:
tunicamycin; tunicamycin analogs; inhibitory anti-Mfsd2A antibodies; and inhibitory nucleic acids.

16. The composition of claim 12, further comprising a central nervous system therapeutic agent.

17.-48. (canceled)
